(19) United States
(12) Patent Application Publication
(10) Pub. No.: US 2024/0174693 A1
(43) Pub. Date: May 30, 2024

(54) SOMATOSTATIN RECEPTOR SUBTYPE 4 (SSTR4) AGONISTS AND THEIR APPLICATIONS

(71) Applicant: Humanwell Pharmaceutical US, Ballwin, MO (US)

(72) Inventors: Shuo Zhao, Ballwin, MO (US); Subo Liao, Ballwin, MO (US); Ming Li, Ballwin, MO (US); Jun Yang, Ballwin, MO (US); Hao Zhou, Ballwin, MO (US); Zejian Ding, Ballwin, MO (US); Xin Zheng, Ballwin, MO (US); Youjian Ning, Ballwin, MO (US); Dali Liang, Ballwin, MO (US); Rong Liu, Ballwin, MO (US); Zewen Liu, Ballwin, MO (US)

(21) Appl. No.: 18/387,694

(22) Filed: Nov. 7, 2023

Related U.S. Application Data
(60) Provisional application No. 63/423,351, filed on Nov. 7, 2022.

(51) Int. Cl. C07D 519/00; A61P 25/04

(52) U.S. Cl. CPC C07D 519/00; A61P 25/04

(57) ABSTRACT

The invention discloses a nitrogen-containing heterocyclic derivative of a somatostatin receptor subtype 4 (SSTR4) small molecule agonist and its pharmaceutical composition, preparation method, and use. The nitrogen-containing heterocyclic derivative of the SSTR4 small molecule agonist is represented by formula (I), and the specific substituents and definitions are as described in the specification. The nitrogen-containing heterocyclic derivatives of SSTR4 small molecule agonists exhibit good binding and agonistic activities with SSTR4. Such compounds or their pharmaceutical compositions have great potential in treating and/or preventing pain disorders related to SSTR4 receptor.

### CROSS-REFERENCE TO RELATED APPLICATION

This application claims benefit of U.S. Provisional Patent Application No. 63/423,351, which was filed in the U.S. Patent and Trademark Office on Nov. 7, 2022, the entire contents of which are incorporated herein by reference for all purposes.

### FIELD OF THE INVENTION

The invention belongs to the field of medicinal chemistry, and specifically relates to nitrogen-containing heterocyclic derivatives of somatostatin receptor subtype 4 (SSTR4) small molecule agonists, their pharmaceutical composition, preparation method and use.

### BACKGROUND OF THE INVENTION

Analgesics are essential drugs for patients with many diseases, as they relieve pain and improve quality of life. As the global population ages and the incidence of chronic diseases such as diabetes, arthritis, joint or bone pain, epilepsy, depression, nerve damage, and various cancers increases, the demand for pain treatment drugs has also significantly increased. Currently, the global pain treatment market is dominated by opioids and nonsteroidal anti-inflammatory drugs (NSAIDS). However, opioids can cause side effects such as respiratory depression, dependence, and constipation, and opioid abuse can also trigger social crises. Therefore, there is a necessity to develop non-addictive analgesics to meet the urgent needs of patients.

Somatostatin, also known as somatotropin release-inhibiting factor, is a cyclic peptide produced by a variety of human organs and tissues. It can act both systemically and locally to inhibit the secretion of various hormones, growth factors, and neurotransmitters (including insulin and glucagon). Somatostatin plays an important role in regulating cell proliferation, glucose homeostasis, inflammation, and pain. The biological properties of somatostatin are mediated through the G protein-coupled receptor family of somatostatin, also known as SSTR or SSI. This family has five subtype receptors, namely SSTR1, SSTR2, SSTR3, SSTR4, and SSTR5.

SSTR4 receptors are distributed in the axons and cell bodies of dorsal root ganglion neurons in rats, monkeys, and humans. It is generally believed that regulating the SSTR4 pathway can alleviate or inhibit pain and inflammation processes. According to recent research, even when opioid drugs fail, somatostatin can relieve pain, playing an important role in neuroregulation, such as pain control mediated by SSTR4 (Chrubasik J, Chrubasik S, Martin E, Acta Neurobiol Exp (Wars), 1993, 53(1): 289-96. Penn R D; Paice J A, Kroin J S, Pain, 1992 April; 49(1): 13-19).

Other research indicates that in the absence of SSTR4, mice are prone to persistent pain and do not experience any analgesic effects (Van Op den Bosch J, et al, J Cell Mol Med, 2009, 13: 3283-3295; Helyes Z. et al, Proc Natl Acad Sci USA. 2009, 106: 13088-13093). Therefore, selective SSTR4 agonists could potentially provide useful therapeutic methods for pain and/or inflammation (Ahmed F. Abdel-Magid, ACS med Chem Lett. 2015, 6, 110). While most SSTR subtypes are involved in regulating homeostatic hormones, SSTR4 appears to play a functional role in modulating sensory neurotransmitters (Pruyank A. Shenoy, Frontiers in Pharm. 2018, vol 9, article 495).

SSTR4 regulates dorsal root ganglion neurons through various mechanisms, such as enhancing potassium currents by opening G protein-coupled inward rectifying potassium channels, reducing calcium currents by inhibiting voltage-gated calcium channels, including transient receptor potential vanilloid-1 and ankyrin 1 channels, thereby controlling pain transmission.

SSTR4 small molecule agonists have been reported in the literatures (Michael Ankersen, Michael Crider, Shengquan Liu, Bin Ho, Henrik S. Andersen, and Carsten Stidsen J. Am. Chem. Soc. 1998, 120, 1368-1373; Mia Engström, Jussi Tomperi, Kamel El-Darwish, Mikaela Åhman, Juha-Matti Savola, and Siegfried Wurster, JPET, 2005, 312:332-338; A. Michael Crider and Ken A. Witt, Mini-Reviews in Medicinal Chemistry, 2007, 7, 213-220). Some of these agonists have demonstrated promising anti-inflammatory and analgesic effects (Boglárka Kántás et al; Int. J. Mol. Sci. 2019, 20, 6245; Eva Szőkea, Neuropharmacology 178 (2020) 108198) Pharmaceutical companies have filed several patents to protect their SSTR4 agonists and explore their potential as new analgesic (U.S. Pat. Nos. 971,282, 9,957,267, 10,166,214, 10,577,336, US20120190691A1, US20180092880A1, WO2021233427A1, WO2021233428A1). Among them, compounds developed by Eli Lilly and Company have entered phase II clinical trials. Recently, the binding structure of SSTR4 receptor with ligands has been reported, which will aid in better ligand design (Wenli Zhao et al. Cell Research, 2022, 0:1-12).

Herein, a class of SSTR4 agonists are provided that may provide effective pain treatment to meet the needs of patients.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The drawings are provided to enhance understanding of the technical aspects of this application and constitute a part of the specification. They are used in conjunction with the embodiments described in this application to illustrate the technical features and should not be considered as limiting the scope of the technical aspects of this application.

FIG. 1A is a graphical representation illustrating the change in swelling degree on one side of a foot before and after modeling over time vs. a model group in an illustrative embodiment of the present disclosure.

FIG. 1B is a graphical representation illustrating the percent change in swelling degree on one side of a foot before and after modeling over time vs. a model group in an illustrative embodiment of the present disclosure.

FIG. 1C is a plot showing the anti-edema efficiency in an illustrative embodiment of the present disclosure.

FIG. 1D shows the chemical structure of a reference compound in an illustrative embodiment of the present disclosure.

FIG. 2A shows a time-effect curve of compound 8 in a CCI model in an illustrative embodiment of the present disclosure.

FIG. 2B is a graphical representation of the data distribution of compound 8 in a CCI model in an illustrative embodiment of the present disclosure.

FIG. 3 shows a concentration vs response curve of compound 8 in hERG in an illustrative embodiment of the present disclosure.

### SUMMARY OF THE INVENTION

In one aspect, disclosed herein are compounds of Formula (I), or its stereoisomer, pharmaceutically acceptable salt, solvate, deuterate, metabolite, or prodrug thereof;

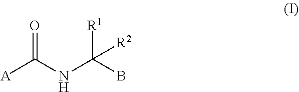

wherein

R¹ and R² are independently selected from H, deuterium, C1-C6 alkyl, substituted C1-C6 alkyl, C3-C6 cycloalkyl, C4-C9 alkylcycloalkyl, C2-C6 alkenyl, substituted C2-C6 alkenyl, aryl, substituted aryl, alkylaryl, or substituted alkylaryl;

wherein the substituted C1-C6 alkyl, substituted C3-C6 cycloalkyl, and substituted aryl are substituted with 1-3 substituents independently selected from the following groups: C1-C3 alkyl, C1-C3 alkoxy, halogen, or halogenated C1-C3 alkyl; or

R¹ and R² are connected to form three-member, four-member, five-member and six-member rings;

A is selected from the following

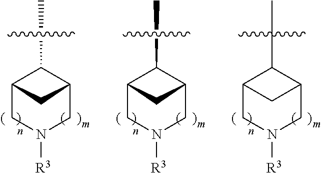

wherein
n=1, 2, 3, 4;
m=1, 2, 3, 4;

R³ is selected from the group consisting of H, deuterium, C1-C6 alkyl, oxygen-containing alkyl, and nitrogen-containing alkyl;

B is selected from the group consisting of aromatic ring, substituted aromatic ring, heterocycle, substituted heterocycle, alkyl heterocycle, and substituted alkyl heterocycle;

wherein the substituted heterocycle or alkyl heterocycle is substituted by 1-3 independently selected from the group consisting of deuterium, halogen, cyano, C1-C6 alkyl, halogenated C1-C6 alkyl, C3-C6 cycloalkyl, C1-C3 alkoxy, alkylsilyl, aryl, substituted aryl, or oxygen, sulfur, selenium containing alkyl.

In yet another aspect, disclosed herein, are pharmaceutical compositions comprising the compounds of Formula (I) or stereoisomer, pharmaceutically acceptable salt, solvate, deuterated compound, metabolite, or prodrug thereof, and a pharmaceutically acceptable carrier.

In still another aspect, disclosed herein are methods of treating and/or preventing a SSTR4 receptor-related disease or disorder, the method comprising administering a pharmaceutically effective amount of the pharmaceutical composition comprising the compounds of Formula (I).

In yet another aspect, disclosed here are methods of making compounds of Formula (I). The method includes reacting a first compound with a protecting group and carboxylic acid group with a second compound with an amine group to form a first intermediate compound; deprotecting the protecting group in the first intermediate compound under acidic conditions to form a second intermediate; and reacting the second intermediate with an amine compound forming the compound of Formula (I).

Other features and iterations of the invention are described in more detail below.

### DETAILED DESCRIPTION OF THE INVENTION

Disclosed herein are a class of SSTR4 agonists, along with their preparation methods and applications. The compounds of the invention exhibit excellent SSTR4 agonistic activity, offering effective pain treatment to meet the needs of patients.

The following is a summary of the subject matter described in detail in this invention. This summary is not intended to limit the scope of the claims for protection.

### (I) Compounds Comprising Formula (I) or Salt Thereof

In one aspect, the present invention provides compounds of formula (I), or their stereoisomers, pharmaceutically acceptable salts, solvates, deuterated forms, metabolites, or prodrugs, as disclosed in the embodiments of the invention.

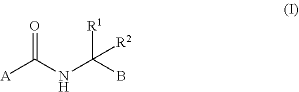

wherein R¹ and R² are independently selected from H, deuterium, C1-C6 alkyl, substituted C1-C6 alkyl, C3-C6 cycloalkyl, C4-C9 alkylcycloalkyl, C2-C6 alkenyl, substituted C2-C6 alkenyl, aryl, and substituted aryl, alkylaryl, substituted alkylaryl. The substituted C1-C6 alkyl, substituted C3-C6 cycloalkyl, and substituted aryl are substituted by 1-3 substituents independently selected from the following groups: C1-C3 alkyl, C1-C3 alkoxy, halogen, halogenated C1-C3 alkyl; R¹ and R² can also be connected to form three-member, four-member, five-member and six-member rings;

A is selected from the following:

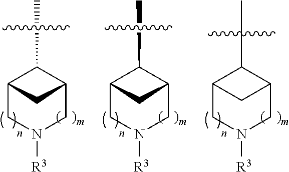

wherein,

n=1, 2, 3, 4;

m=1, 2, 3, 4;

R³ is selected from the group consisting of H, deuterium, C1-C6 alkyl, oxygen-containing alkyl, and nitrogen-containing alkyl;

B is selected from the group consisting of aromatic ring, substituted aromatic ring, heterocycle, substituted heterocycle, alkyl heterocycle, and substituted alkyl heterocycle; the substituted heterocycle or alkyl heterocycle is substituted by 1-3 independently selected from the group consisting of halogen, cyano, C1-C6 alkyl, halogenated C1-C6 alkyl, C3-C6 cycloalkyl, C1-C3 alkoxy, alkylsilyl, aryl, substituted aryl, and oxygen, sulfur, selenium containing alkyl.

In some embodiments, the compound comprises the formula (Ia)

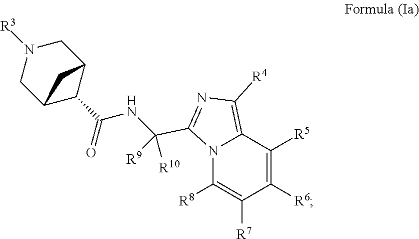

wherein

R³ is hydrogen or amidine;

R⁴-R⁸ are independently hydrogen, halogen, C1-C6 alkyl, substituted C1-C6 alkyl, C1-C3 alkoxy, C3-C6 cycloalkyl, aryl, substituted aryl, sulfinyl, substituted sulfinyl, or C1-C3 alkyl silyl group; and

R³-R⁸ are independently hydrogen or C1-C6 alkyl.

In some embodiments, the compound comprises the formula (Ib)

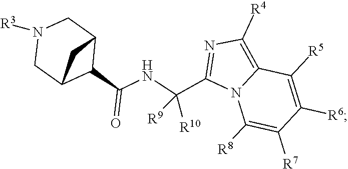

wherein

R³ is hydrogen or amidine;

R⁴-R⁸ are independently hydrogen, halogen, C1-C6 alkyl, substituted C1-C6 alkyl, C1-C3 alkoxy, C3-C6 cycloalkyl, aryl, substituted aryl, sulfinyl, substituted sulfinyl, or C1-C3 alkyl silyl group; and

R³-R⁸ are independently hydrogen or C1-C6 alkyl.

Specifically, in some embodiments, the present invention provides a compound represented by formula (I), and its stereoisomers, pharmaceutically acceptable salts, solvates, deuterates, metabolites, or prodrugs;

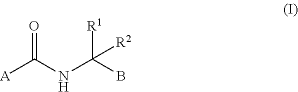

wherein R¹ and R² are independently selected from H, deuterium, C1-C6 alkyl, substituted C1-C6 alkyl, C3-C6 cycloalkyl, C4-C9 alkylcycloalkyl, C2-C6 alkenyl, substituted C2-C6 alkenyl, aryl, and substituted aryl, alkylaryl, substituted alkylaryl;

the substituted C1-C6 alkyl, substituted C3-C6 cycloalkyl, and substituted aryl are substituted by 1-3 substituents independently selected from the following groups: C1-C3 alkyl, C1-C3 alkoxy, halogen, halogenated C1-C3 alkyl; R¹ and R² can also be connected to form three-member, four-member, five-member and six-member rings;

A is selected from the following:

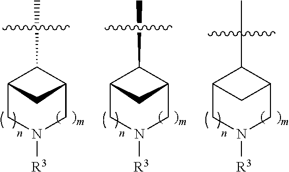

wherein,

n=1, 2, 3, 4;

m=1, 2, 3, 4;

R³ is selected from the group consisting of H, deuterium, C1-C6 alkyl, oxygen-containing alkyl, and nitrogen-containing alkyl;

B is selected from the group consisting of aromatic ring, substituted aromatic ring, heterocycle, substituted heterocycle, alkyl heterocycle, and substituted alkyl heterocycle; the substituted heterocycle or alkyl heterocycle is substituted by 1-3 independently selected from the group consisting of halogen, cyano, C1-C6 alkyl, halogenated C1-C6 alkyl, C3-C6 cycloalkyl, C1-C3 alkoxy, alkylsilyl, aryl, substituted aryl, and oxygen, sulfur, selenium containing alkyl.

In some embodiments, R¹ and R² in formula (I) are independently selected from H, deuterium, methyl, ethyl, propyl, butyl, substituted C1-C6 alkyl, C3-C6 cycloalkyl, C4-C9 alkylcycloalkyl, C2-C6 alkenyl, substituted C2-C6 alkenyl, aryl, substituted aryl, alkylaryl, and substituted alkylaryl.

In some embodiments, R¹ and R² in formula (I) are independently selected from H, deuterium, methyl, ethyl, propyl, butyl, cyclopropyl, cyclobutyl, cyclopentyl, cyclopropylmethyl, cyclopropylethyl, C2-C6 alkenyl, substituted C2-C6 alkenyl, aryl, substituted aryl, alkylaryl, and substituted alkylaryl.

In some embodiments, R¹ and R² in formula (I) are independently selected from H, deuterium, methyl, ethyl, propyl, butyl, cyclopropyl, cyclobutyl, cyclopentyl, cyclopropylmethyl, cyclopropylethyl, ethenyl, propenyl, isopropenyl, phenyl, naphthyl, phenylmethyl, and phenylethyl.

In some embodiments, A in formula (I) is selected from the following:

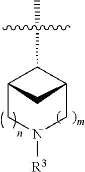

wherein, n=1, m=1.

In some embodiments, A in formula (I) is selected from the following:

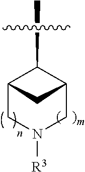

wherein, n=1, m=1.

In some embodiments, A in formula (I) is selected from the following:

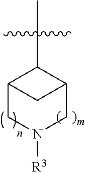

wherein, n=1, m=1.

In some embodiments, R³ in formula (I) is independently selected from H, deuterium, methyl, ethyl, propyl, butyl, oxygen-containing alkyl, and nitrogen-containing alkyl.

In some embodiments, R³ in formula (I) is independently selected from H, deuterium, methyl, ethyl, propyl, butyl, aldehyde, ester, carboxyl, and nitrogen-containing alkyl.

In some embodiments, R³ in formula (1) is independently selected from H, deuterium, methyl, ethyl, propyl, butyl, aldehyde, ester, carboxyl, methylamino, formylamide, and amidine.

In some embodiments, B in formula (I) is selected from phenyl, pyridine, imidazole, and pyrimidine fused with pyridine.

In some embodiments, B in formula (I) is selected from substituted aromatic rings, substituted heterocyclic rings, and substituted alkyl heterocycles. The substituted aromatic rings, heterocyclic rings, and alkyl heterocycles are independently substituted with 1-3 substituents selected from the following groups: fluorine, chlorine, bromine, cyano, methyl, ethyl, propyl, butyl, trifluoromethyl, cyclopropyl, cyclobutyl, cyclopentyl, methoxy, ethoxy, (3,3-dimethylbutyl)oxy, trimethylsilyl, phenyl, 3-methoxyphenyl, and sulfonyl.

Furthermore, the compounds of formula (I) are selected from the following compounds 1-23, as well as their stereoisomers, pharmaceutically acceptable salts, solvates, deuterated forms, metabolites, or prodrugs:

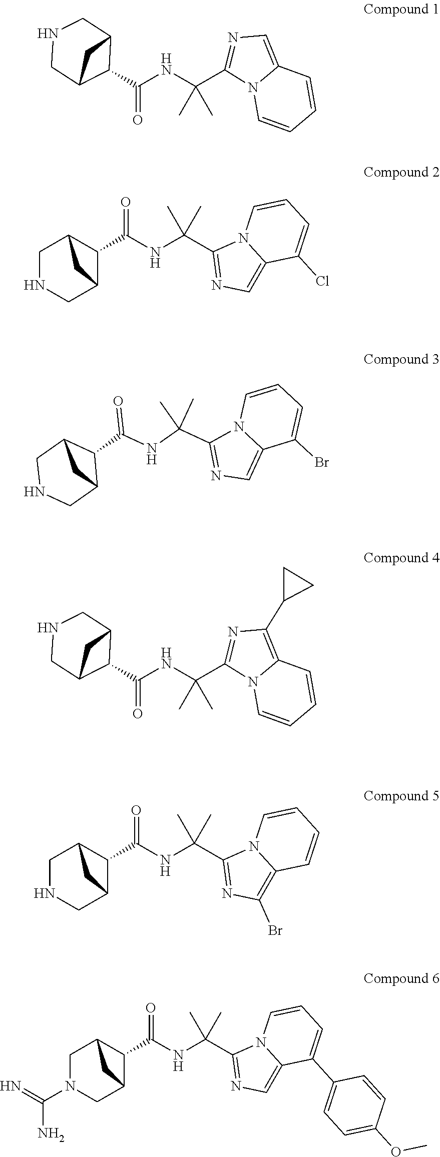 [SMILES: CC(C)(NC(=O)[C@@H]1[C@@H]2CNC[C@H]1C2)c1ncc2ccccn12 ; CC(C)(NC(=O)[C@@H]1[C@@H]2CNC[C@H]1C2)c1ncc2c(Cl)cccn12 ; CC(C)(NC(=O)[C@@H]1[C@@H]2CNC[C@H]1C2)c1ncc2c(Br)cccn12 ; CC(C)(NC(=O)[C@@H]1[C@@H]2CNC[C@H]1C2)c1nc(C2CC2)c2ccccn12 ; CC(C)(NC(=O)[C@@H]1[C@@H]2CNC[C@H]1C2)c1nc(Br)c2ccccn12 ; COc1ccc(-c2cccn3c(C(C)(C)NC(=O)[C@H]4[C@@H]5C[C@H]4CN(C(=N)N)C5)ncc23)cc1]

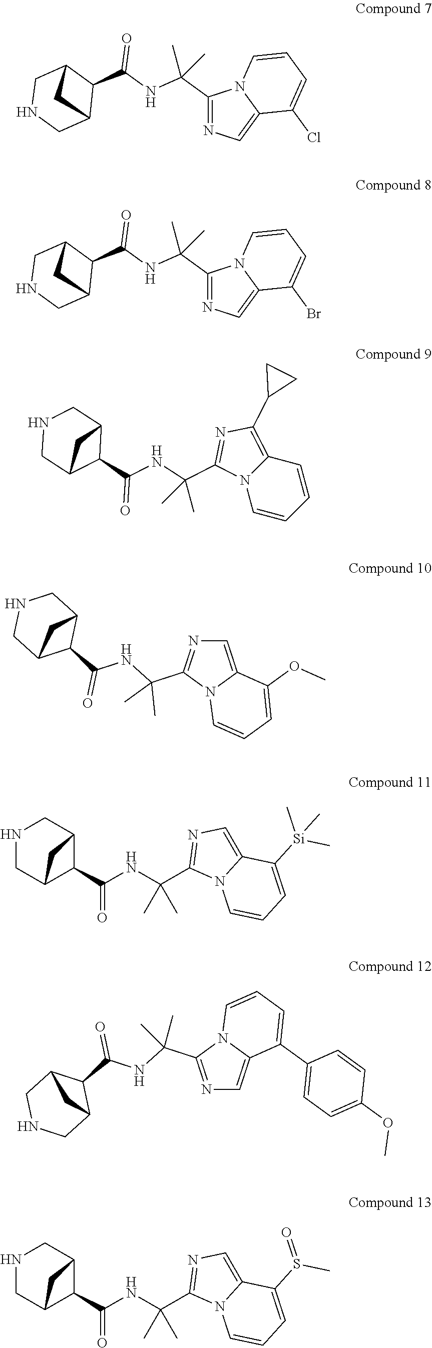

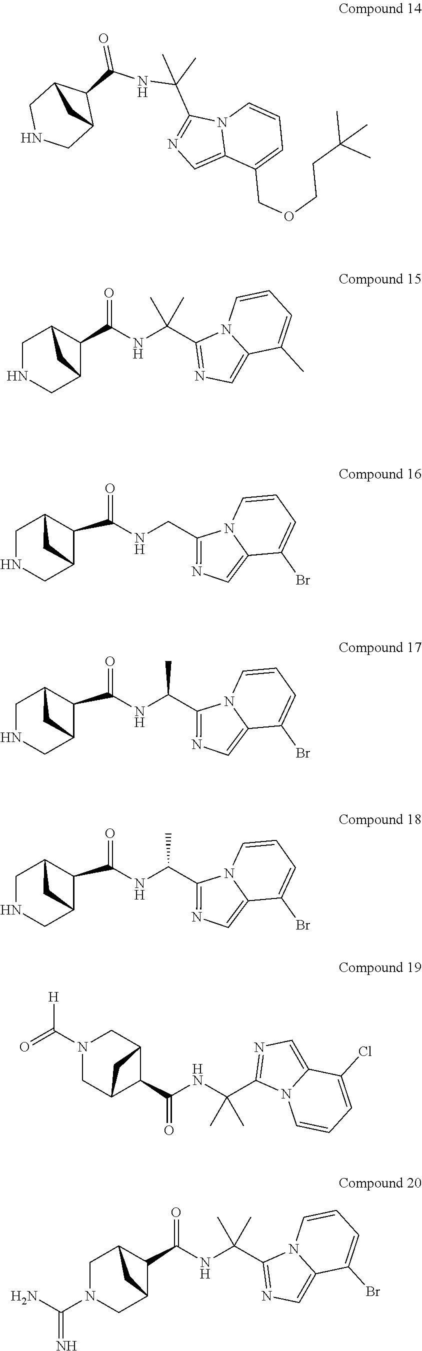

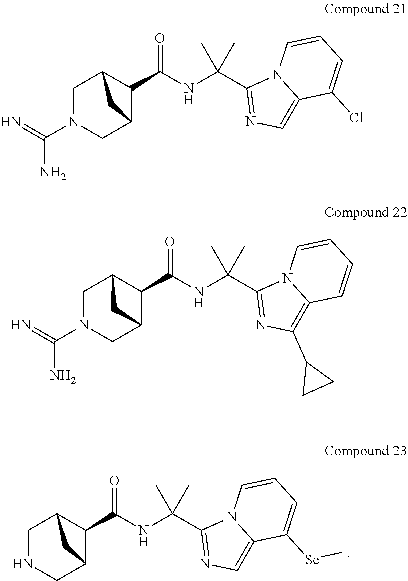

### (II) Processes for Preparing Compounds Having Formula (I)

In some aspects, the present application provides a method for preparing the above compound or its stereoisomers, pharmaceutically acceptable salts, solvates, deuterates, metabolites, or prodrugs.

The preparation method comprises the following steps:

(1) conduction of a condensation reaction between compound (I-1) and compound (I-2) to obtain compound (I-3);

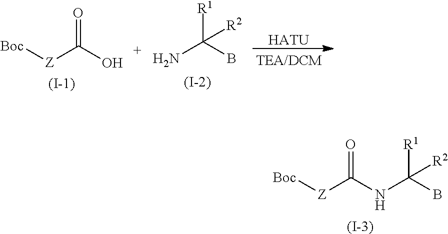

(2) Compound (I-3) is subjected to deprotection of the Boc group under acidic conditions to obtain compound (I-4) or the compound of formula (I);

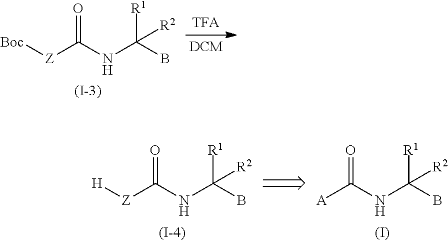

(3) Compound (I-4) reacts with compound (I-5) under alkaline conditions to yield compound (I-6), i.e., the compound of formula (I);

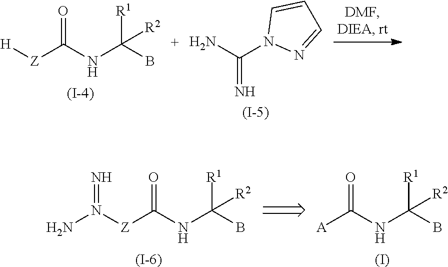

(4) Compound (I-7) undergoes a condensation reaction with compound (I-2) to yield compound (I-8), i.e., the compound of formula (I);

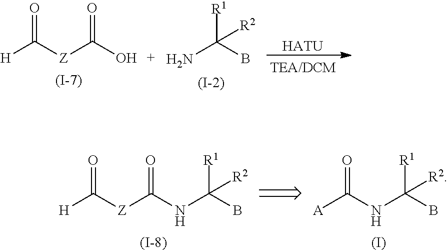

### (III) Compositions Comprising Compounds of Formula (I)

In certain embodiments, the present invention provides a pharmaceutical composition comprising compounds of formula (I) and their stereoisomers, pharmaceutically acceptable salts, solvates, deuterated forms, metabolites, or prodrugs, along with at least one pharmaceutically acceptable excipient.
(a) Compounds of formula (I) and its stereoisomers, pharmaceutically acceptable salts, solvates, deuterates, metabolites, or prodrugs.
(b) Excipient

The disclosed pharmaceutical composition comprises at least one pharmaceutically acceptable excipient. Non-limiting examples of suitable excipients may include diluents, binders, fillers, buffering agents, pH-adjusting agents, disintegrants, dispersants, stabilizers, preservatives, and colorants. The quantity and type of excipients can be selected based on established pharmaceutical principles.

The pharmaceutical composition can be blended with one or more excipients to create solid, liquid, or cream formulations. The methods for preparing solid, liquid, or cream formulations are well-established in the field.

### (IV) Methods of Use

On another aspect, the present invention provides a method for treating SSTR4 receptor-related diseases or disorders. The method involves administering to an individual in need thereof a pharmaceutical composition comprising a compound of formula (I).

Without being constrained by any particular theory, the compound of formula (I) is believed to primarily function as an agonist that mediates the activity of the SSTR4 receptor. Binding at this site is considered to have the potential to treat conditions such as pain, addiction, depression, stress, anxiety, autoimmune diseases, or neurological disorders.

These compounds can be administered through various routes. For example, the compound of formula (I) can be administered orally through solid or liquid formulations (tablets, gel caps, sustained-release capsule powders, solutions, or suspensions in aqueous or non-aqueous liquids), parenterally (including subcutaneous, intradermal, intravenous injections—either as a solution, suspension, or emulsion in a carrier), or topically (such as transdermal or transmucosal administration, including but not limited to oral, rectal, vaginal, and sublingual administration).

In one embodiment, the compound can be administered in physiological saline or in combination with the aforementioned pharmaceutically acceptable excipients. The compound can be administered as the primary therapy or adjunct therapy, administered post-local intervention (surgery, radiation therapy, local chemotherapy), or co-administered with at least one other chemotherapeutic agent.

Appropriate subjects for study can include but are not limited to humans and companion animals such as cats, dogs, rodents, and horses; research animals such as rabbits, sheep, pigs, dogs, primates, mice, rats, and other rodents; farm animals such as cows, cattle, pigs, goats, sheep, horses, deer, chickens, and other poultry; zoo animals; and primates such as chimpanzees, monkeys, and gorillas. The age of the subjects is not limited. In a preferred embodiment, the experimental subjects can be humans.

In general, the compound of formula (I) will be administered in a therapeutically effective amount, which includes prophylactic amounts or lower doses, for example, when co-administered with another formulation. The term “effective amount” as used herein refers to a dose of the compound sufficient to provide a circulating concentration high enough to exert a beneficial effect on the receptors. Skilled practitioners can determine the precise dosage based on the desired amount, side effects, and the patient's medical history.

In general, the compound of formula (I) exhibits an EC₅₀ for binding affinity to the SSTR4 receptor of less than about 100 nM. In various embodiments, the compounds comprising one of formulae (1), (II), (III), (IV), or (V) have an EC₅₀ of less than about 100 nM, or less than 10 nM, or less than about 5 nM, or less than about 1 nM.

In general, the compound of formula (I) exhibits an EC₅₀ in CAMP assays in whole cells of less than 100 nM. In various embodiments, the compound of formula (I) has an EC₅₀ of less than about 100 nM, or less than 10 nM, or less than about 5 nM, or less than about 1 nM.

### Beneficial Effects

The compounds of the present invention demonstrate a remarkable ability to bind to the SSTR4 receptor, exhibiting robust agonistic activity on the SSTR4 receptor. These compounds are well-suited for pharmaceutical applications and have significant clinical utility. Additionally, the synthetic process for the compounds disclosed in this application is straightforward, contributing to their considerable economic value.

### Definition and Explanation of Terms

Unless otherwise stated, the groups and terms defined in this specification and claims, including instances, exemplary definitions, preferred definitions, as described in the tables, and those specifically defined for compounds in the examples, can be freely combined and interchanged. Subsequent definitions of groups and compound structures should fall within the scope of what is disclosed in the specification.

The compounds described herein may have asymmetric centers. The compounds of the present invention containing asymmetrically substituted atoms can be separated into optical isomers or racemates. Unless specifically indicated for a particular stereochemical or isomeric form, all chiral, non-enantiomeric, racemic forms, and all geometric isomeric forms of the structure are contemplated.

The term “alkyl” as used herein refers to a lower alkyl having 1 to 6 carbon atoms on the main chain, and up to 20 carbon atoms in total. These can be straight-chain, branched, or cyclic, including methyl, ethyl, propyl, isopropyl, butyl, hexyl, and so on.

The term “aromatic” as used herein, either alone or as part of another moiety, refers to any optionally substituted mono- or heterocyclic, conjugated, planar ring system containing delocalized electrons. Preferred aromatic moieties include single rings (such as furan or benzene), fused rings, or tricyclic moieties with a ring portion containing 5-14 atoms. The term “aromatic” encompasses the definition of “aryl” as defined below.

The term “aryl” or “Ar” as used herein, either alone or as part of another moiety, refers to any optionally substituted monocyclic or bicyclic aromatic group, preferably a single ring or fused ring system with 6 to 10 carbon atoms in the ring portion. Examples include phenyl, biphenyl, naphthyl, substituted phenyl, substituted biphenyl, or substituted naphthyl.

The term “carbocycle” or “carbocyclic” as used herein, either alone or as part of another moiety, refers to any optionally substituted, aromatic or non-aromatic, monocyclic or polycyclic ring system in which all atoms in the ring are carbon, preferably having 5 or 6 carbon atoms in each ring. Exemplary substituents may include one or more of the following groups: alkyl, substituted alkyl, alkane, alkoxy, acyl, acyloxy, alkenyl, alkenoxy, aryl, aryloxy, amino, amido, formyl, aminoformyl, carbocycle, cyano, ester, ether, halogen, heterocycle, hydroxy, ketone, enone, phosphoric acid, nitro, and thiol.

The term “heteroaromatic ring” as used herein, either alone or as part of another moiety, refers to an optionally substituted aromatic moiety having at least one heteroatom in at least one ring, preferably having 5 or 6 atoms in each ring. Preferred heteroaromatic ring moieties have 1 or 2 oxygen atoms and/or 1 to 4 nitrogen atoms in each ring, and are attached to the rest of the molecule via carbon. Exemplary moieties include furan, benzofuran, oxazole, isoxazole, oxadiazole, benzoxazole, benzodiazole, pyrrole, pyrazole, imidazole, triazole, tetrazole, pyridine, quinoline, pyrimidine, diazine, indole, isoindole, indazole, benzimidazole, indolizine, benzotriazole, carbazole, pyrrolopyridine, quinoxaline, quinazoline, and quinoline. Exemplary substituents may include one or more of the following groups: alkyl, substituted alkyl, alkane, alkoxy, acyl, acyloxy, alkene, alkenoxy, aryl, aryloxy, amino, acylamino, formyl, aminoformyl, carbocycle, cyano, ester, ether, halogen, heterocycle, hydroxy, keto, ketoenol, phosphoric, nitro, and thio.

The term “heterocycle” or “heterocyclic” as used herein, either alone or as part of another moiety, refers to bicyclic, aromatic or non-aromatic moiety having at least one heteroatom, preferably having 5 or 6 atoms in each ring. Preferred heterocycle moieties have 1 or 2 oxygen atoms and/or 1 to 4 nitrogen atoms in each ring, and are attached to the rest of the molecule via carbon or a heteroatom. Exemplary heterocycle moieties include the aforementioned heteroaromatic ring compounds. Exemplary substituents may include one or more of the following groups: alkyl, substituted alkyl, alkane, alkoxy, acyl, acyloxy, alkene, alkenoxy, aryl, aryloxy, amino, acylamino, formyl, aminoformyl, carbocycle, cyano, ester, ether, halogen, heterocycle, hydroxy, keto, ketoenol, phosphoric, nitro, and thio.

The term “protecting group” as used in this document refers to a group that can protect a specific portion, where the protecting group can be removed after the protection reaction without interfering with the rest of the molecule. When the protected portion is an oxygen atom (forming a protected hydroxy group), exemplary protecting groups include ethers (such as allyl, triphenylmethyl (trityl or Tr), benzyl, para-methoxybenzyl (PMB), para-methylphenyl (PMP)), acetals (such as methoxymethyl (MOM), beta-methoxyethoxymethyl (MEM), tetrahydropyran (THP), ethoxyethyl (EE), methylthiomethyl (MTM), 2-methoxy-2-propyl (MOP), 2-trimethylsilyl ethoxymethyl (SEM)), esters (such as benzoyl (Bz), vinyl carbonate, trichloroethyl carbonate (Troc), 2-trimethylsilyl carbonate), and methylsilane ethers (such as trimethylsilyl (TMS), triethylsilyl (TES), triisopropylsilyl (TIPS), triphenylsilyl (TPS), tert-butyldimethylsilyl (TBDMS), tert-butyldiphenylsilyl (TBDPS)), and so on. When the protected portion is a nitrogen atom (thus forming a protected amine), exemplary protecting groups include benzyl (such as para-methoxybenzyl (PMP), 3,4-dimethoxybenzyloxy (PMB)), esters (such as benzoyl (Bz)), carbonyl (such as para-methoxybenzyl carbonyl (Moz), tert-butoxycarbonyl (BOC), 9-fluorenylmethoxycarbonyl (FMOC)), acetyl, formylamino, N-methylsilyl, and so on. Various protecting groups and their synthesis methods can be referred to in “Greene's Protective Groups in Organic Synthesis” (Fourth Edition) co-authored by P. G. M. Wuts and T. W. Greene, John Wiley & Sons, Inc.

The “substituted hydrocarbon group” as described in this document is a hydrocarbon portion that is substituted by at least one non-carbon atom, including portions where carbon chain atoms are substituted by heteroatoms such as nitrogen, oxygen, silicon, phosphorus, boron, or halogen atoms, and portions where the carbon chain contains additional substituents. These substituents include alkyl, alkoxy, acyl, acyloxy, alkenyl, alkenyloxy, aryl, aryloxy, amino, acylamino, aldehyde, formylamino, carbocyclyl, cyano, ester, ether, halogen, heterocyclyl, hydroxy, keto, ketal, phosphino, nitro, and thio groups.

The terms “comprise,” “include,” and “have” are inclusive and mean that additional elements may be present besides the listed elements. After describing the present invention in detail, it is apparent that modifications and variations can be made without departing from the scope of the invention as defined in the appended claims. Other features and advantages of the present invention will be elucidated in the subsequent description, and, in part, become apparent or are understood through the implementation of the present invention. The objectives and other advantages of the present invention can be achieved and obtained through the structures particularly pointed out in the specification, claims, and drawings.

### EXAMPLES

The following examples illustrate various embodiments of the invention. The following will provide a more detailed explanation of the general formula compounds, their preparation methods, and applications of the present invention, with specific examples. The examples presented below are intended for illustrative purposes and to explain the invention, without being construed as limitations on the scope of the protection afforded by the present invention. All technologies based on the content described herein that are achieved fall within the scope intended to be protected by the present invention.

Unless otherwise stated, the raw materials and reagents used in the following examples are commercially available or can be prepared by known methods. This application uses the following abbreviations: DCM: Dichloromethane; DMF: N,N-Dimethylformamide; EA: Ethyl acetate; EtOH: Ethanol; HATU: Hexafluorophosphate Azabenzotriazole Tetramethyl Uronium; Na₂SO₄: Sodium sulfate; NaHSO₄: Sodium bisulfate; and TEA: Triethylamine. The compounds are named according to conventional nomenclature rules in the field, and commercially available reagents are referred to by the supplier's catalog names.

The ¹H NMR data were collected and recorded using a Bruker Avance Neo 500 MHz liquid-state superconducting nuclear magnetic resonance spectrometer at 500 MHz. CDCl₃ was used as the solvent, and TMS (δ=0) served as the internal standard to report chemical shifts (δ values) in ppm. Mass spectrometry was performed using a Waters ACQUITY UPLC system with an ACQUITY UPLC BEH C8, 50 mm*2.1 mm, 1.7 μm (20180306-C8-08) chromatography column. The mobile phase A consisted of 0.01% TFA/H₂O, and mobile phase B was CH₃CN; the flow rate was 0.2 mL/min, the column temperature was 30° C., and the detection wavelength was UV-210 nm. High-Performance Liquid Chromatography (HPLC) was conducted using a Thermo UltiMate 3000 liquid chromatography system with a Venusil ASB C18 (4.6*250 mm, 5 μm) chromatography column. The mobile phase A was a phosphate solution with pH=1.5, and mobile phase B was CH₃CN. The flow rate was 1.0 mL/min, the column temperature was 35° C., and the detection wavelength was UV-215 nm. The injection volume was 2 μL. The gradient elution conditions were as follows: the elution was performed at a flow rate of 1.0 mL/min, starting with 95% A and 5% B for 10 minutes, followed by a 5-minute wash with 20% A and 80% B, and finally, a 5-minute wash with 95% A and 5% B. The percentages indicate the volume percentages of the mobile phase in the elution solution.

### Example 1: Synthesis of Compound 1

The synthetic route of (1R,5S,6s)-N-(2-(imidazo[1,5-a]pyridin-3-yl)propan-2-yl)-3-azabicyclo[3.1.1]heptane-6-carboxamide Compound 1 is shown in Reaction Scheme 1:

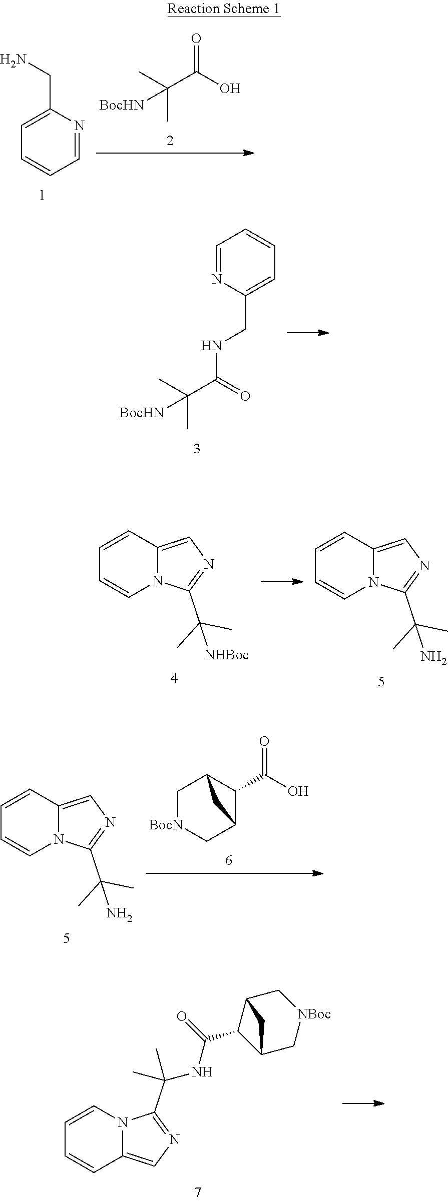

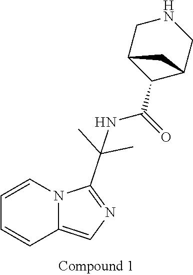 [SMILES: CC(C)(NC(=O)[C@@H]1[C@@H]2CNC[C@H]1C2)c1ncc2ccccn12]

Step 1: Synthesis of tert-butyl (2-methyl-1-oxo-1-((pyridin-2-ylmethyl)amino)propan-2-yl)carbamate 3

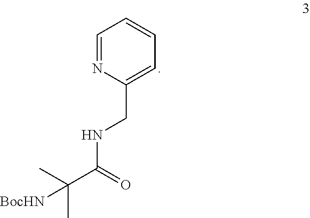 [SMILES: CC(C)(C)OC(=O)NC(C)(C)C(=O)NCc1ccccn1]

2 g of pyridin-2-ylmethylamine 1 (18.5 mmol) and 20 mL of DCM (solvent) were added to a 100 mL round-bottom flask. To the mixture, 2-(tert-butoxycarbonylamino)-2-methylpropanoic acid 2 (3.95 g, 19.4 mmol) and HATU (8.44 g, 22.2 mmol) were added, and TEA (10 mL) was finally added. The reaction mixture was stirred at room temperature for 1 hour. LC-MS indicated the completion of the reaction of the starting materials. The mixture was extracted with DCM and a NaHSO₄ solution. The organic layer was dried over anhydrous Na₂SO₄, concentrated, and tert-butyl (2-methyl-1-oxo-1-((pyridin-2-ylmethyl)amino)propan-2-yl)carbamate 3 (5.4 g, 98%) was obtained. MS m/z: [M+H]⁺ 294.18.

Step Two: Synthesis of tert-butyl (2-(imidazo[1,5-a]pyridin-3-yl)propan-2-yl)carbamate 4

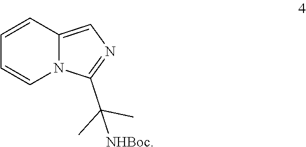 [SMILES: CC(C)(C)OC(=O)NC(C)(C)c1ncc2ccccn12]

Tert-butyl (2-methyl-1-oxo-1-((pyridin-2-ylmethyl)amino)propan-2-yl)carbamate 3 (5.4 g, 18.4 mmol) was weighed into a 250 mL round-bottom flask and dissolved in 60 mL DCM. An appropriate amount of molecular sieves were added and stirred for 30 min. Burgess reagent (6.58 g, 27.6 mmol) was added to the mixture, and stirring was continued for 2 hours. LC-MS indicated the complete reaction of the starting materials. The mixture was filtered, and the filtrate was directly used for column chromatography purification. The product tert-butyl (2-(imidazo[1,5-a]pyridin-3-yl)propan-2-yl)carbamate 4 (2 g, 98.5%) was obtained. MS m/z: [M+H]⁺ 276.71.

Step 3: Synthesis of 2-(imidazo[1,5-a]pyridin-3-yl)propan-2-amine 5

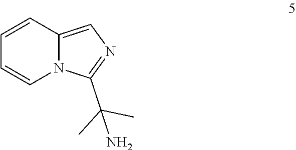

In a 50 mL round-bottom flask, tert-butyl (2-(imidazo[1,5-a]pyridin-3-yl)propan-2-yl)carbamate 4 (2 g, 1.3 mmol) was combined with EtOH/HCl (10 mL). The mixture was stirred at room temperature for 30 minutes, followed by vacuum distillation. This process yielded 2-(imidazo[1,5-a]pyridin-3-yl)propan-2-amine 5 (1.8 g, 99%). The product was characterized by MS (m/z): [M+H]⁺ 176.12.

Step 4: Synthesis of tert-butyl (1R,5S,6s)-6-((2-(imidazo[1,5-a]pyridin-3-yl)propan-2-yl)carbamoyl)-3-azabicyclo[3.1.1]heptane-3-carboxylate 7.

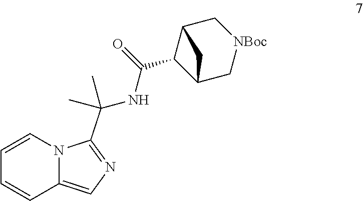 [SMILES: CC(C)(C)OC(=O)N1C[C@H]2C[C@@H](C1)[C@H]2C(=O)NC(C)(C)c1ncc2ccccn12]

2-(imidazo[1,5-a]pyridin-3-yl)propan-2-amine 5 (1.8 g, 10.2 mmol) and 20 mL DCM (solvent) were added to a 100 mL round-bottom flask. (1R,5S,6s)-3-(tert-butoxycarbonyl)-3-azabicyclo[3.1.1]heptane-6-carboxylic acid 6 (2.59 g, 10.71 mmol) and HATU (4.65 g, 12.24 mmol) were added to the mixture. Finally, TEA (10 mL) was added and the reaction mixture stirred at room temperature for 1 hour. LC-MS confirmed the complete reaction of the starting materials. The mixture was extracted with DCM and NaHSO₄ solution. The organic layer was dried with anhydrous Na₂SO₄, concentrated, and purified by column chromatography to obtain tert-butyl (1R,5S,6s)-6-((2-(imidazo[1,5-a]pyridin-3-yl)propan-2-yl)carbamoyl)-3-azabicyclo[3.1.1]heptane-3-carboxylate (4.0 g, 97%). MS m/z: [M+H]⁺ 399.24 7.

Step 5: Synthesis of (1R,5S,6s)-N-(2-(imidazo[1,5-a]pyridin-3-yl)propan-2-yl)-3-azabicyclo[3.1.1]heptane-6-carboxamide (Compound 1)

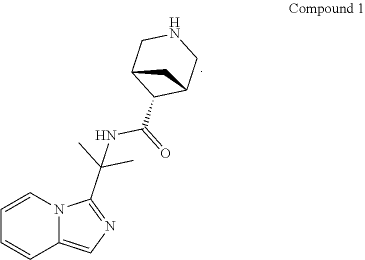 [SMILES: CC(C)(NC(=O)[C@@H]1[C@@H]2CNC[C@H]1C2)c1ncc2ccccn12]

In a 50 mL round-bottom flask, tert-butyl (1R,5S,6s)-6-((2-(imidazo[1,5-a]pyridin-3-yl)propan-2-yl)carbamoyl)-3-azabicyclo[3.1.1]heptane-3-carboxylate 7 (4.0 g, 10 mmol) and EtOH/HCl (20 mL) were added. Stir at room temperature for 30 min, then perform vacuum distillation to obtain (1R,5S,6s)-N-(2-(imidazo[1,5-a]pyridin-3-yl)propan-2-yl)-3-azabicyclo[3.1.1]heptane-6-carboxamide (2.98 g, 99%) Compound 1. ¹H NMR (500 MHZ, Chloroform-d) δ 8.75 (dd, J =7.4, 1.5 Hz, 1H), 8.17 (s, 1H), 7.75-7.68 (m, 1H), 7.34-7.26 (m, 1H), 7.01 (s, 1H), 6.99-6.92 (m, 1H), 3.12-3.00 (m, 5H), 2.71 (t, J=5.8 Hz, 1H), 2.49-2.40 (m, 2H), 1.95-1.87 (m, 1H), 1.77-1.68 (m, 1H), 1.69 (s, 5H), 1.65-1.55 (m, 1H). MS m/z: [M+H]⁺ 299.19; HPLC>95%.

### Example 2: Synthesis of Compound 2

The synthetic route of (1R,5S,6s)-N-(2-(8-chloroimidazo[1,5-a]pyridin-3-yl)propan-2-yl)-3-azabicyclo[3.1.1]heptane-6-carboxamide Compound 2 is shown in Reaction Scheme 2.

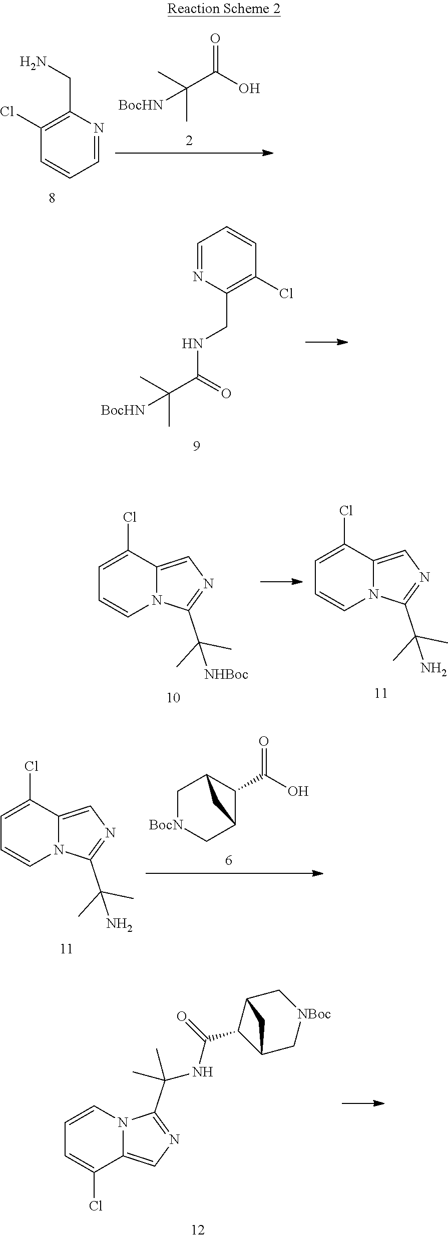

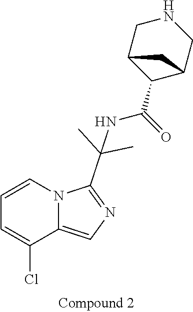 [SMILES: CC(C)(NC(=O)[C@@H]1[C@@H]2CNC[C@H]1C2)c1ncc2c(Cl)cccn12]

Step 1: Synthesis of tert-butyl (1-(((3-chloropyridin-2-yl)methyl)amino)-2-methyl-1-oxopropan-2-yl)carbamate 9

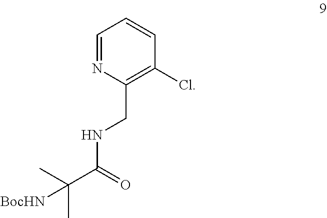 [SMILES: CC(C)(C)OC(=O)NC(C)(C)C(=O)NCc1ncccc1Cl]

(3-chloropyridin-2-yl)methanamine 8 (2 g, 14 mmol) and 20 mL DCM as a solvent were added into a 100 mL round-bottom flask. 2-(tert-butoxycarbonylamino)-2-methylpropanoic acid 2 (3.42 g, 16.8 mmol) and HATU (6.38 g, 16.8 mmol) were added to the mixture, and finally, TEA (10 mL) was added. The mixture was stirred at room temperature for 1 hour. LC-MS indicated the complete reaction of the starting materials. The mixture was extracted with DCM and a NaHSO₄ solution. The organic layer was dried with anhydrous Na₂SO₄, concentrated, and tert-butyl (1-(((3-chloropyridin-2-yl)methyl)amino)-2-methyl-1-oxopropan-2-yl)carbamate 9 (4.59 g, 100%) was obtained. MS m/z: [M+H]+ 328.14.

Step 2: Synthesis of tert-butyl (2-(8-chloroimidazo[1,5-a]pyridin-3-yl)propan-2-yl)carbamate 10

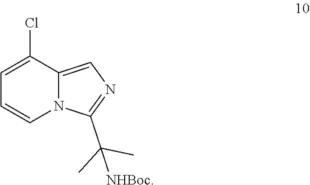 [SMILES: CC(C)(C)OC(=O)NC(C)(C)c1ncc2c(Cl)cccn12]

Tert-butyl (1-(((3-chloropyridin-2-yl)methyl)amino)-2-methyl-1-oxopropan-2-yl)carbamate 9 (4.59 g, 14 mmol) was added to a 250 mL round-bottom flask. 60 mL DCM was added to the flask to dissolve the tert-butyl (1-(((3-chloropyridin-2-yl)methyl)amino)-2-methyl-1-oxopropan-2-yl)carbamate. An appropriate amount of molecular sieves were added and stirred for 30 min, then Burgess reagent (5.0 g, 21 mmol) was added to the mixture. The mixture was stirred for 2 h. LC-MS indicated complete reaction of the starting materials. The mixture was filtered, and the filtrate was directly subjected to flash column chromatography to obtain the product tert-butyl (2-(8-chloroimidazo[1,5-a]pyridin-3-yl)propan-2-yl)carbamate 10 (1.3 g, 30%). MS m/z: [M+H]+ 310.13.

Step 3: Synthesis of 2-(8-chloroimidazo[1,5-a]pyridin-3-yl)propan-2-amine 11

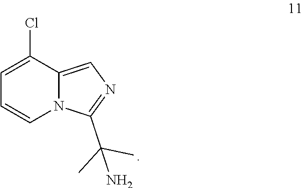

In a 50 mL round-bottom flask, tert-butyl (2-(8-chloroimidazo[1,5-a]pyridin-3-yl)propan-2-yl)carbamate 10 (1.3 g, 4.2 mmol) and EtOH/HCl (10 mL) were added. The mixture was stirred at room temperature for 30 minutes, followed by vacuum distillation to obtain 2-(8-chloroimidazo[1,5-a]pyridin-3-yl)propan-2-amine 11 (790 mg, 90%). MS m/z: [M+H]⁺ 210.08.

Step 4: Synthesis of tert-butyl (1R,5S,6s)-6-((2-(8-chloroimidazo[1,5-a]pyridin-3-yl)propan-2-yl)carbamoyl)-3-azabicyclo[3.1.1]heptane-3-carboxylate 12

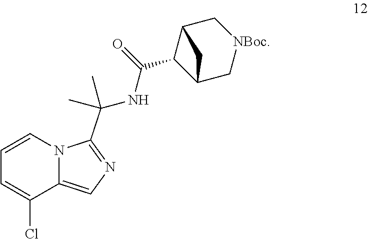 [SMILES: CC(C)(C)OC(=O)N1C[C@H]2C[C@@H](C1)[C@H]2C(=O)NC(C)(C)c1ncc2c(Cl)cccn12]

2-(8-chloroimidazo[1,5-a]pyridin-3-yl)propan-2-amine 11 (790 mg, 3.8 mmol) was placed into a 100 mL round-bottom flask, DCM as the solvent was added, and sequentially (1R,5S,6s)-3-(tert-butoxycarbonyl)-3-azabicyclo[3.1.1 ]heptane-6-carboxylic acid 6 (1.0 g, 4.2 mmol) and HATU (1.75 g, 4.6 mmol) were introduced to the mixture. TEA (10 mL) was finally added. The mixture was stirred at room temperature for 1 hour. LC-MS analysis confirmed the completion of the reaction for the starting materials. The mixture was extracted with DCM and NaHSO₄ solution, the organic layer was dried with anhydrous Na₂SO₄, concentrated, and purified by column chromatography to yield tert-butyl (1R,5S,6s)-6-((2-(8-chloroimidazo[1,5-a]pyridin-3-yl)propan-2-yl)carbamoyl)-3-azabicyclo[3.1.1]heptane-3-carboxylate (1.64 g, 100%) 12. MS m/z: [M+H]⁺ 433.20.

Step 5: Sysnthesis of (1R,5S,6s)-N-(2-(8-chloroimidazo[1,5-a]pyridin-3-yl)propan-2-yl)-3-azabicyclo[3.1.1]heptane-6-carboxamide 12.

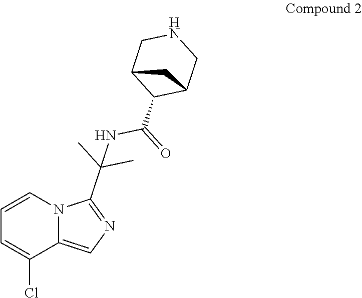 [SMILES: CC(C)(NC(=O)[C@@H]1[C@@H]2CNC[C@H]1C2)c1ncc2c(Cl)cccn12]

Tert-butyl (1R,5S,6s)-6-((2-(8-chloroimidazo[1,5-a]pyridin-3-yl)propan-2-yl)carbamoyl)-3-azabicyclo[3.1.1]heptane-3-carboxylate 12 (1.64 g, 3.8 mmol) and EtOH/HCl (20 mL) were added to a 50 mL round-bottom flask. The mixture was stirred at room temperature for 30 minutes, followed by vacuum distillation, yielding (1R,5S,6s)-N-(2-(8-chloroimidazo[1,5-a]pyridin-3-yl)propan-2-yl)-3-azabicyclo[3.1.1]heptane-6-carboxamide Compound 2 (1.1 g, 90%). ¹H NMR (500 MHz, Chloroform-d) δ 9.18 (dd, J=7.3, 1.3 Hz, 1H), 8.26 (s, 1H), 7.59 (dd, J=7.4, 1.5 Hz, 1H), 7.11 (t, J=7.3 Hz, 1H), 6.98 (s, 1H), 3.11-3.04 (m, 5H), 2.71 (t, J=5.8 Hz, 1H), 2.48-2.40 (m, 3H), 1.94-1.87 (m, 1H), 1.76-1.68 (m, 1H), 1.64-1.56 (m, 1H). MS m/z: [M+H]⁺ 333.15; HPLC>95%.

### Example 3: Synthesis of Compound 3

The synthetic route of (1R,5S,6s)-N-(2-(8-bromoimidazo[1,5-a]pyridin-3-yl)propan-2-yl)-3-azabicyclo[3.1.1]heptane-6-carboxamide Compound 3 is shown in Reaction Scheme 3.

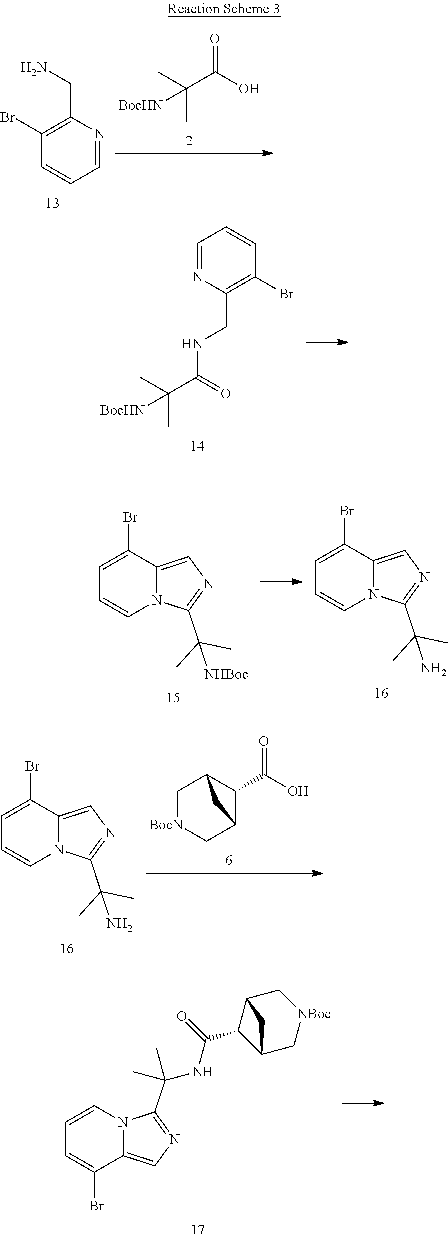

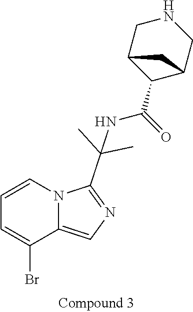 [SMILES: CC(C)(NC(=O)[C@@H]1[C@@H]2CNC[C@H]1C2)c1ncc2c(Br)cccn12]

Step 1: Synthesis of tert-butyl (1-(((3-bromopyridin-2-yl)methyl)amino)-2-methyl-1-oxopropan-2-yl)carbamate 14.

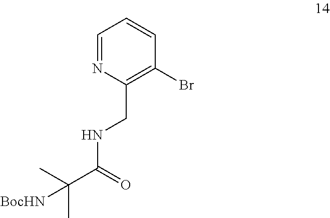 [SMILES: CC(C)(C)OC(=O)NC(C)(C)C(=O)NCc1ncccc1Br]

3-bromo-2-pyridinylmethanamine 13 (2 g, 10.7 mmol) and 20 ml of DCM as the solvent were added to a 100 mL round-bottom flask. To the mixture, 2-(tert-butoxycarbonylamino)-2-methylpropanoic acid 2 (2.6 g, 12.8 mmol) and HATU (4.86 g, 12.8 mmol) were added. Finally, TEA (10 mL) was added and stirred at room temperature for 1 hour. LC-MS showed complete reaction of the starting material. The mixture was extracted with a DCM and NaHSO₄ solution. The organic layer was dried over anhydrous Na₂SO₄, and the solution was concentrated to obtain tert-butyl (1-(((3-bromopyridin-2-yl)methyl)amino)-2-methyl-1-oxopropan-2-yl)carbamate 14 (3.9 g, 100%). MS m/z: [M+H]⁺ 372.09.

Step 2: Synthesis of tert-butyl (2-(8-bromoimidazo[1,5-a]pyridin-3-yl)propan-2-yl)carbamate 15.

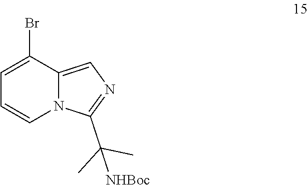 [SMILES: CC(C)(C)OC(=O)NC(C)(C)c1ncc2c(Br)cccn12]

tert-butyl (1-(((3-bromopyridin-2-yl)methyl)amino)-2-methyl-1-oxopropan-2-yl)carbamate 14 (3.9 g, 10.7 mmol) was weighed and added to a 250 mL round-bottom flask. The tert-butyl (1-(((3-bromopyridin-2-yl)methyl)amino)-2-methyl-1-oxopropan-2-yl)carbamate 14 was dissolved in 60 mL of DCM. An appropriate amount of molecular sieves were added and stirred for 30 minutes. Then, Burgess reagent (3.8 g, 16 mmol) was added to the mixture and stirredfor 2 hours. LC-MS showed complete reaction of the starting material. The mixture was filtered and the filtrate was used for column chromatography to obtain the product tert-butyl (2-(8-bromoimidazo[1,5-a]pyridin-3-yl)propan-2-yl)carbamate 15 (1.1g, 30%). MS m/z: [M+H]⁺ 354.08.

Step 3: Synthesis of 2-(8-bromoimidazo[1,5-a]pyridin-3-yl)propan-2-amine 16.

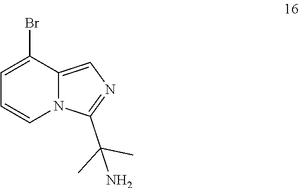

tert-butyl (2-(8-bromoimidazo[1,5-a]pyridin-3-yl)propan-2-yl)carbamate 15 (1.1 g, 3.2 mmol) was added to a 50 mL round-bottom flask. EtOH/HCl (5 mL) was added to the mixture, stirred at room temperature for 30 minutes, and then vacuum distillation was performed to obtain 2-(8-bromoimidazo[1,5-a]pyridin-3-yl)propan-2-amine 16 (740 mg, 90%).ss MS m/z: [M+H]⁺ 254.03.

Step 4: Synthesis of tert-butyl (1R,5S,6s)-6-((2-(1-bromoimidazo[1,5-a]pyridin-3-yl)propan-2-yl)carbamoyl)-3-azabicyclo[3.1.1]heptane-3-carboxylate 17.

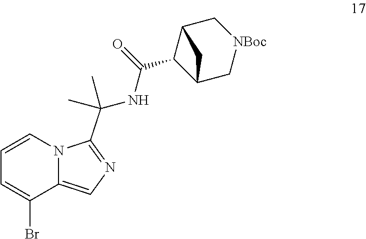 [SMILES: CC(C)(C)OC(=O)N1C[C@H]2C[C@@H](C1)[C@H]2C(=O)NC(C)(C)c1ncc2c(Br)cccn12]

2-(8-bromoimidazo[1,5-a]pyridin-3-yl)propan-2-amine 16 (740 mg, 2.9 mmol) was added to a 100 mL round-bottom flask. 10 mL of DCM as the solvent was added to the mixture. (1R,5S,6s)-3-(tert-butoxycarbonyl)-3-azabicyclo[3.1.1]heptane-6-carboxylic acid 6 (770 mg, 3.2 mmol) and HATU (1.3 g, 3.5 mmol) were added to the mixture. Finally, add TEA (5 mL) was added and stirred at room temperature for 1 hour. LC-MS showed complete reaction of the starting material. Extract the mixture with DCM and NaHSO₄ solution. The organic layer was dried over anhydrous Na₂SO₄, and the solution was concentrated. The product was purified by column chromatography to obtain tert-butyl (1R,5S,6s)-6-((2-(1-bromoimidazo[1,5-a]pyridin-3-yl)propan-2-yl)carbamoyl)-3-azabicyclo[3.1.1]heptane-3-carboxylate 17 (1.1 g, 80%). MS m/z: [M+H]⁺ 477.15.

Step 5: Synthesis of (1R,5S,6s)-N-(2-(8ss-bromoimidazo[1,5-a]pyridin-3-yl)propan-2-yl)-3-azabicyclo[3.1.1]heptane-6-carboxamide (Compound 3).

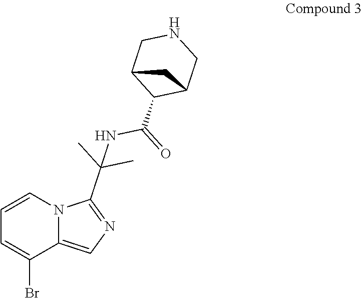 [SMILES: CC(C)(NC(=O)[C@@H]1[C@@H]2CNC[C@H]1C2)c1ncc2c(Br)cccn12]

tert-butyl (1R,5S,6s)-6-((2-(1-bromoimidazo[1,5-a]pyridin-3-yl)propan-2-yl)carbamoyl)-3-azabicyclo[3.1.1]heptane-3-carboxylate 17 (1.1 g, 2.3 mmol) was added to a 50 mL round-bottom flask. EtOH/HCl (10 mL) was added and stirred at room temperature for 30 minutes. Vacuum distillation was performed to obtain (1R,5S,6s)-N-(2-(8-bromoimidazo[1,5-a]pyridin-3-yl)propan-2-yl)-3-azabicyclo[3.1.1]heptane-6-carboxamide Compound 3 (790 mg, 90%). ¹H NMR (500 MHZ, Chloroform-d) δ 9.25 (dd, J=7.3, 1.3 Hz, 3H), 8.11 (s, 3H), 7.69 (dd, J=7.4, 1.5 Hz, 3H), 7.14 (t, J=7.3 Hz, 3H), 6.98 (s, 3H), 3.11-3.04 (m, 11H), 3.03 (dd, J=4.2, 2.9 Hz, 1H), 2.71 (t, J=5.8 Hz, 3H), 2.48-2.40 (m, 6H), 1.94-1.87 (m, 3H), 1.76-1.68 (m, 21H), 1.64-1.56 (m, 3H). MS m/z: [M+H]⁺ 377.10; HPLC>95%.

### Example 4: Synthesis of Compound 4

The synthetic route of (1R,5S,6s)-N-(2-(1-cyclopropylimidazo[1,5-a]pyridin-3-yl)propan-2-yl)-3-azabicyclo[3.1.1]heptane-6-carboxamide Compound 4 is shown in Reaction Scheme 4.

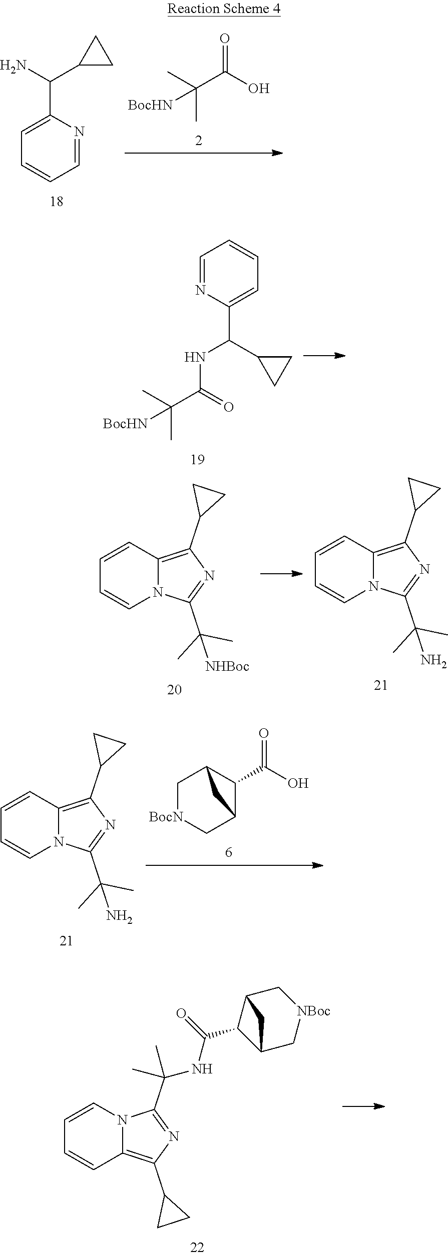

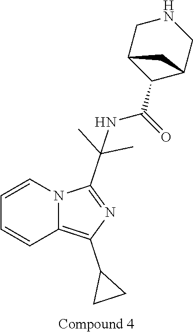 [SMILES: CC(C)(NC(=O)[C@@H]1[C@@H]2CNC[C@H]1C2)c1nc(C2CC2)c2ccccn12]

Step 1: Synthesis of tert-butyl (1-((cyclopropyl(pyridin-2-yl)methyl)amino)-2-methyl-1-oxopropan-2-yl)carbamate 19.

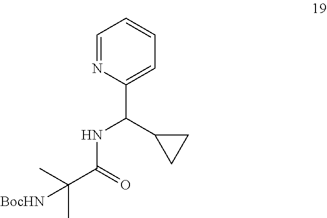 [SMILES: CC(C)(C)OC(=O)NC(C)(C)C(=O)NC(c1ccccn1)C1CC1]

Cyclopropyl(pyridin-2-yl)methanamine 18 (2 g, 13.5 mmol) was placed into a 100 mL round-bottom flask, 20 mL of DCM added as the solvent, and sequentially 2-(tert-butoxycarbonylamino)-2-methylpropanoic acid 2 (3.3 g, 16.2 mmol) and HATU (6.16 g, 16.2 mmol) were introduced to the mixture. Finally, TEA (10 mL) was added. The mixture was stirred at room temperature for 1 hour. LC-MS analysis indicated the completion of reaction of the starting materials. The mixture was extracted with DCM and NaHSO₄ solution, the organic layer dried with anhydrous Na₂SO₄, and concentrated to obtain tert-butyl (1-((cyclopropyl(pyridin-2-yl)methyl)amino)-2-methyl-1-oxopropan-2-yl)carbamate 19 (4.5 g, 100%). MS m/z: [M+H]⁺ 334.21.

Step 2: Synthesis of tert-butyl (2-(1-cyclopropylimidazo[1,5-a]pyridin-3-yl)propan-2-yl)carbamate 20.

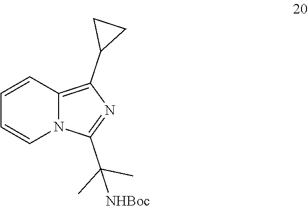 [SMILES: CC(C)(C)OC(=O)NC(C)(C)c1nc(C2CC2)c2ccccn12]

The compound tert-butyl (1-((cyclopropyl(pyridin-2-yl)methyl)amino)-2-methyl-1-oxopropan-2-yl)carbamate 19 (4.5 g, 13.5 mmol) was added to a 250 ml round-bottom flask, dissolved in 60 mL of DCM, with the addition of an appropriate amount of molecular sieves. The mixture was stirred for 30 minutes, followed by the addition of Burgess reagent (4.8 g, 20 mmol), and the stirring continued for an additional 2 hours. LC-MS indicated complete reaction of the starting material. The mixture was filtered, and the filtrate was directly subjected to column chromatography for purification to obtain the tert-butyl (2-(1-cyclopropylimidazo[1,5-a]pyridin-3-yl)propan-2-yl)carbamate 20 (1.28 g, 30%). MS m/z: [M+H]⁺316.20.

Step 3: Synthesis of 2-(1-cyclopropylimidazo[1,5-a]pyridin-3-yl)propan-2-amine 21.

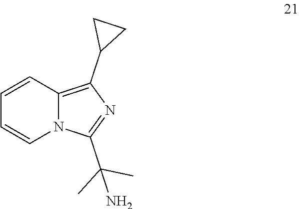 [SMILES: CC(C)(N)c1nc(C2CC2)c2ccccn12]

In a 50 mL round-bottom flask, tert-butyl (2-(1-cyclopropylimidazo[1,5-a]pyridin-3-yl)propan-2-yl)carbamate 20 (1.28 g, 4.05 mmol) and EtOH/HCl (10 mL) were added. The mixture was stirred at room temperature for 30 minutes, followed by vacuum distillation to obtain 2-(1-cyclopropylimidazo[1,5-a]pyridin-3-yl)propan-2-amine 21 (775 mg, 89%). MS m/z: [M+H]⁺ 216.15.

Step 4: Synthesis of tert-butyl (1R,5S,6s)-6-((2-(1-cyclopropylimidazo[1,5-a]pyridin-3-yl)propan-2-yl)carbamoyl)-3-azabicyclo[3.1.1]heptane-3-carboxylate 22.

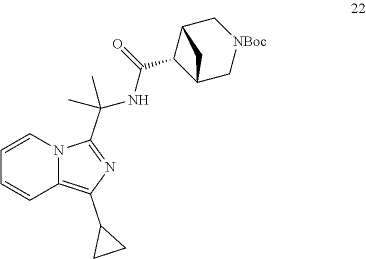 [SMILES: CC(C)(C)OC(=O)N1C[C@H]2C[C@@H](C1)[C@H]2C(=O)NC(C)(C)c1nc(C2CC2)c2ccccn12]

2-(1-cyclopropylimidazo[1,5-a]pyridin-3-yl)propan-2-amine 21 (790 mg, 3.6 mmol) was transferred to a 100 mL round-bottom flask and 20 mL of DCM was added as a solvent. To the mixture, (1R,5S,6s)-3-(tert-butoxycarbonyl)-3-azabicyclo[3.1.1]heptane-6-carboxylic acid 6 (965 mg, 4.0 mmol) and HATU (1.6 g, 4.3 mmol) were added. Finally, TEA (10 mL) was added and stirred at room temperature for 1 hour. LC-MS showed complete reaction of the starting material. The mixture was extracted with DCM and NaHSO₄ solution. The organic layer was dried over anhydrous Na₂SO₄, concentrated, and purified by column chromatography to obtain tert-butyl (1R,5S,6s)-6-((2-(1-cyclopropylimidazo[1,5-a]pyridin-3-yl)propan-2-yl)carbamoyl)-3-azabicyclo[3.1.1]heptane-3-carboxylate 22 (1.49 g, 94%). MS m/z: [M+H]⁺ 439.27.

Step 5: Synthesis of (1R,5S,6s)-N-(2-(1-cyclopropylimidazo[1,5-a]pyridin-3-yl)propan-2-yl)-3-azabicyclo[3.1.1]heptane-6-carboxamide Compound 4.

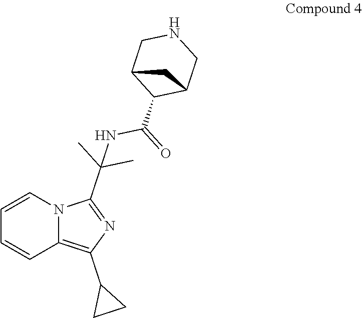 [SMILES: CC(C)(NC(=O)[C@@H]1[C@@H]2CNC[C@H]1C2)c1nc(C2CC2)c2ccccn12]

In a 50 mL round-bottom flask, tert-butyl (1R,5S,6s)-6-((2-(1-cyclopropylimidazo[1,5-a]pyridin-3-yl)propan-2-yl)carbamoyl)-3-azabicyclo[3.1.1]heptane-3-carboxylate 22 (1.49 g, 3.4 mmol) and EtOH/HCl (20 mL) were added. The mixture was stirred at room temperature for 30 minutes, followed by vacuum distillation to obtain (1R,5S,6s)-N-(2-(1-cyclopropylimidazo[1,5-a]pyridin-3-yl)propan-2-yl)-3-azabicyclo[3.1.1]heptane-6-carboxamide Compound 4 (1.05 g, 88%). ¹H NMR (500 MHZ, Chloroform-d) δ 8.82 (dd, J=7.2, 1.3 Hz, 1H), 7.63 (dd, J=9.6, 1.5 Hz, 1H), 7.23-7.15 (m, 1H), 7.06-6.96 (m, 2H), 3.12-3.00 (m, 4H), 2.84-2.75 (m, 1H), 2.71 (t, J=5.8 Hz, 1H), 2.49-2.40 (m, 2H), 1.95-1.87 (m, 1H), 1.72 (s, 6H), 1.77-1.68 (m, 1H), 1.65-1.55 (m, 1H), 1.08-0.94 (m, 4H). MS m/z: [M+H]⁺ 339.22; HPLC>95%.

### Example 5: Synthesis of Compound 5

The synthetic route of (1R,5S,6s)-N-(2-(1-bromoimidazo[1,5-a]pyridin-3-yl)propan-2-yl)-3-azabicyclo[3.1.1]heptane-6-carboxamide Compound 5 is shown in Reaction scheme 5.

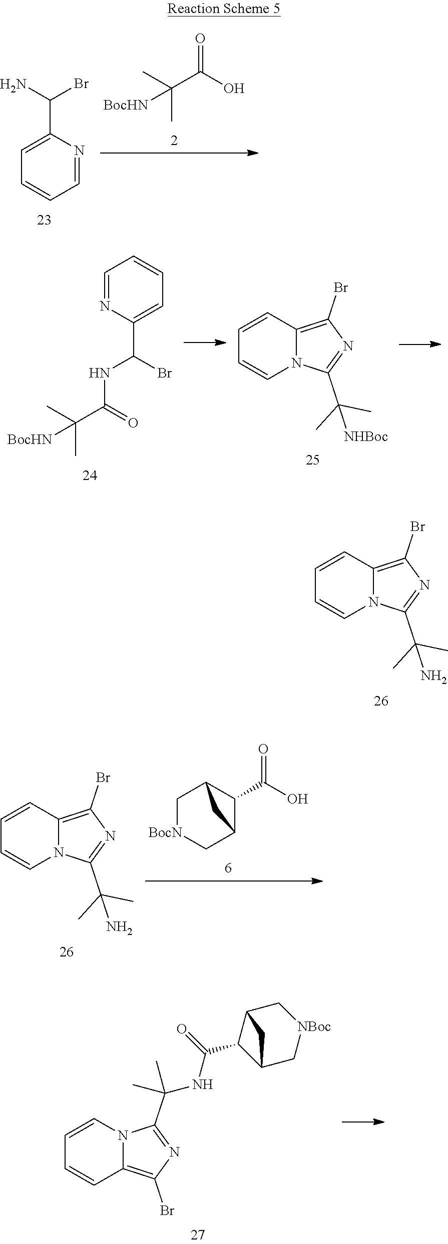

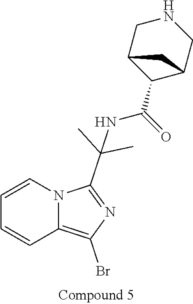 [SMILES: CC(C)(NC(=O)[C@@H]1[C@@H]2CNC[C@H]1C2)c1nc(Br)c2ccccn12]

Step 1: Synthesis of tert-butyl (1-((bromo(pyridin-2-yl)methyl)amino)-2-methyl-1-oxopropan-2-yl)carbamate 24.

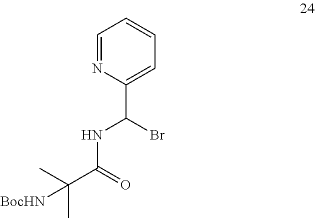 [SMILES: CC(C)(C)OC(=O)NC(C)(C)C(=O)NC(Br)c1ccccn1]

20 mL of DCM was added as a solvent to bromo(pyridin-2-yl)methanamine 23 (2 g, 10.7 mmol) in a 100 mL round-bottom flask. To the mixture, 2-(tert-butoxycarbonylamino)-2-methylpropanoic acid 2 (2.6 g, 12.8 mmol) and HATU (4.86 g, 12.8 mmol) were added. Finally, TEA (10 mL) was added and stirred at room temperature for 1 hour. LC-MS showed complete reaction of the starting material. Extract the mixture with a DCM and NaHSO₄ solution. The organic layer was dried over anhydrous Na₂SO₄, and the solution was concentrated to obtain tert-butyl (1-((bromo(pyridin-2-yl)methyl)amino)-2-methyl-1-oxopropan-2-yl)carbamate 24 (3.9 g, 100%). MS m/z: [M+H]⁺ 372.09.

Step 2: Synthesis of tert-butyl (2-(1-bromoimidazo[1,5-a]pyridin-3-yl)propan-2-yl)carbamate 25.

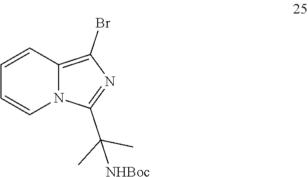 [SMILES: CC(C)(C)OC(=O)NC(C)(C)c1nc(Br)c2ccccn12]

tert-butyl (1-((bromo(pyridin-2-yl)methyl)amino)-2-methyl-1-oxopropan-2-yl)carbamate 24 (3.9 g, 10.7 mmol) was weighed and added to a 250 mL round-bottom flask and dissolved in 60 mL of DCM. An appropriate amount of molecular sieves was added, and stirred for 30 minutes. Then, Burgess reagent (3.8 g, 16 mmol) was added to the mixture and stirred for an additional 2 hours. LC-MS indicated complete reaction of the starting material. The mixture was filtered, and the filtrate was directly subjected to column chromatography for purification to obtain the product tert-butyl (2-(1-bromoimidazo[1,5-a]pyridin-3-yl)propan-2-yl)carbamate 25 (1.1 g, 30%). MS m/z: [M+H]⁺ 352.08.

Step 3: Synthesis of 2-(1-bromoimidazo[1,5-a]pyridin-3-yl)propan-2-amine

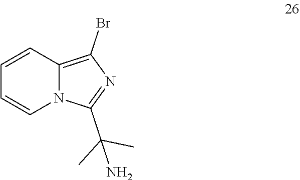

In a 50 mL round-bottom flask, tert-butyl (2-(1-bromoimidazo[1,5-a]pyridin-3-yl)propan-2-yl)carbamate 25 (1.1 g, 3.2 mmol) and EtOH/HCl (5 mL) were added. The mixture was stirred at room temperature for 30 minutes, followed by vacuum distillation to obtain 2-(1-bromoimidazo[1,5-a]pyridin-3-yl)propan-2-amine 26 (740 mg, 90%). MS m/z: [M+H]⁺254.03.

Step 4: Synthesis of tert-butyl (1R,5S,6s)-6-((2-(1-bromoimidazo[1,5-a]pyridin-3-yl)propan-2-yl)carbamoyl)-3-azabicyclo[3.1.1]heptane-3-carboxylate 27.

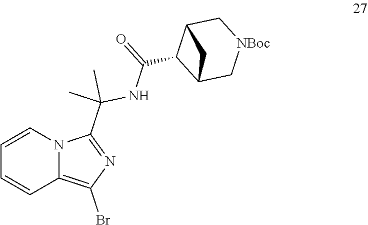 [SMILES: CC(C)(C)OC(=O)N1C[C@H]2C[C@@H](C1)[C@H]2C(=O)NC(C)(C)c1nc(Br)c2ccccn12]

2-(1-bromoimidazo[1,5-a]pyridin-3-yl)propan-2-amine 26 (740 mg, 2.9 mmol) was added to a 100 mL round-bottom flask. Next 10 mL of DCM was added to the flask as the solvent. To the mixture, (1R,5S,6s)-3-(tert-butoxycarbonyl)-3-azabicyclo[3.1.1]heptane-6-carboxylic acid 6 (770 mg, 3.2 mmol) and HATU (1.3 g, 3.5 mmol) were added. Finally, TEA (5 mL) was added and stirred at room temperature for 1 hour. LC-MS showed complete reaction of the starting material. The mixture was extracted with a DCM and NaHSO₄ solution. The organic layer was dried over anhydrous Na₂SO₄, and the solution was concentrated to obtain tert-butyl (1R,5S,6s)-6-((2-(1-bromoimidazo[1,5-a]pyridin-3-yl)propan-2-yl)carbamoyl)-3-azabicyclo[3.1.1]heptane-3-carboxylate 27 (1.1 g, 80%). MS m/z: [M+H]⁺ 477.15.

Step 5: Synthesis of (1R,5S,6s)-N-(2-(1-bromoimidazo[1,5-a]pyridin-3-yl)propan-2-yl)-3-azabicyclo[3.1.1]heptane-6-carboxamide Compound 5.

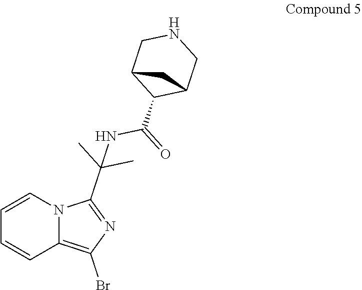 [SMILES: CC(C)(NC(=O)[C@@H]1[C@@H]2CNC[C@H]1C2)c1nc(Br)c2ccccn12]

In a 50 mL round-bottom flask, tert-butyl (1R,5S,6s)-6-((2-(1-bromoimidazo[1,5-a]pyridin-3-yl)propan-2-yl)carbamoyl)-3-azabicyclo[3.1.1]heptane-3-carboxylate 27 (1.1 g, 2.3 mmol) and EtOH/HCl (10 mL) were added. The mixture was stirred at room temperature for 30 minutes, followed by vacuum distillation to obtain (1R,5S,6s)-N-(2-(1-bromoimidazo[1,5-a]pyridin-3-yl)propan-2-yl)-3-azabicyclo[3.1.1]heptane-6-carboxamide Compound 5 (790 mg, 90%). ¹H NMR (500 MHz, Chloroform-d) δ 8.83 (dd, J=7.4, 1.5 Hz, 3H), 7.77 (dd, J=8.9, 1.4 Hz, 3H), 7.35-7.28 (m, 3H), 7.06-6.96 (m, 6H), 3.11-3.04 (m, 11H), 3.03 (dd, J=4.2, 2.9 Hz, 1H), 2.71 (t, J=5.8 Hz, 3H), 2.48-2.40 (m, 6H), 1.94-1.87 (m, 3H), 1.75-1.68 (m, 2H), 1.64-1.56 (m, 3H). MS m/z: [M+H]⁺ 377.10; HPLC>95%.

### Example 6: Synthesis of Compound 6

The synthetic route of (1R,5S,6r)-3-carbamimidoyl-N-(2-(8-(4-methoxyphenyl)imidazo[1,5-a]pyridin-3-yl)propan-2-yl)-3-azabicyclo[3.1.1]heptane-6- carboxamide Compound 6 is shown in Reaction Scheme 6.

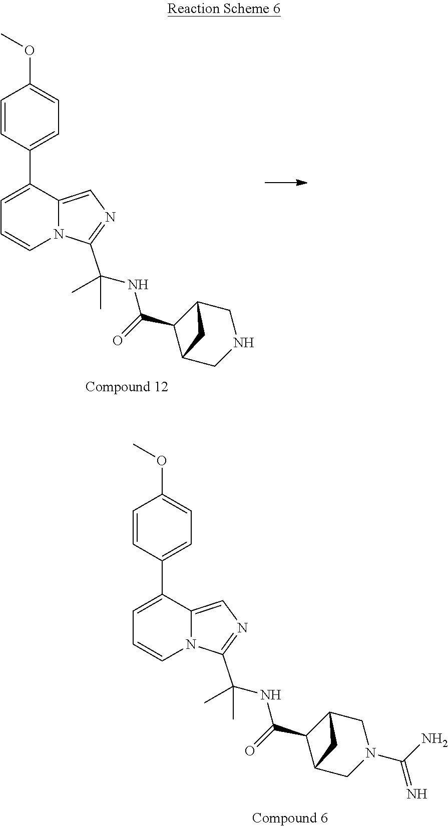 [SMILES: COc1ccc(-c2cccn3c(C(C)(C)NC(=O)[C@H]4[C@@H]5CNC[C@H]4C5)ncc23)cc1 ; COc1ccc(-c2cccn3c(C(C)(C)NC(=O)[C@@H]4[C@@H]5C[C@H]4CN(C(=N)N)C5)ncc23)cc1]

Step 1: Synthesis of (1R,5S,6r)-3-carbamimidoyl-N-(2-(8-(4-methoxyphenyl)imidazo[1,5-a]pyridin-3-yl)propan-2-yl)-3-azabicyclo[3.1.1]heptane-6- carboxamide Compound 6

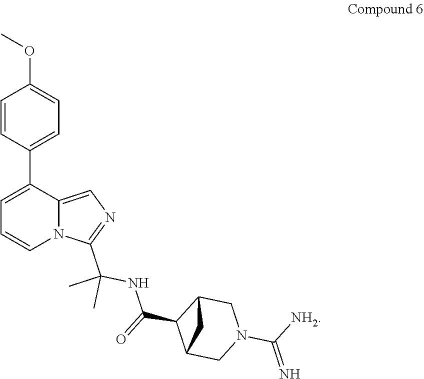 [SMILES: COc1ccc(-c2cccn3c(C(C)(C)NC(=O)[C@@H]4[C@@H]5C[C@H]4CN(C(=N)N)C5)ncc23)cc1]

(1R,5S,6r)-N-(2-(8-(4-methoxyphenyl)imidazo[1,5-a]pyridin-3-yl)propan-2-yl)-3-azabicyclo[3.1.1]heptane-6-carboxamide Compound 12 (500 mg, 1.2 mmol) was added to a 100 mL round-bottom flask. 1H-pyrazole-1-methanol hydrochloride (210 mg, 1.4 mmol) and DIPEA (4 mL) were added to the flask. The mixture was stirred at room temperature for 1 hour. The completion of the reaction was confirmed by LC-MS. The mixture was extracted with DCM and NaHSO₄ solution. The organic layer was dried over anhydrous Na₂SO₄, concentrated, and purified by column chromatography to obtain (1R,5S,6r)-3-carbamimidoyl-N-(2-(8-(4-methoxyphenyl)imidazo[1,5-a]pyridin-3-yl)propan-2-yl)-3-azabicyclo[3.1.1]heptane-6- carboxamide Compound 6 (322 mg, 60%). ¹H NMR (500 MHz, Chloroform-d) δ 9.18 (dd, J=7.2, 1.4 Hz, 1H), 8.21 (s, 1H), 7.86 (dd, J=6.9, 1.5 Hz, 1H), 7.46-7.40 (m, 2H), 7.28 (s, 1H), 7.10 (t, J=7.1 Hz, 1H), 6.97 (d, J=12.6 Hz, 1H), 6.97-6.92 (m, 2H), 5.51 (s, 2H), 3.82 (s, 2H), 3.72 (dd, J=12.2, 1.6 Hz, 2H), 3.57 (dd, J=12.4, 1.6 Hz, 2H), 2.64 (t, J=6.7 Hz, 1H), 2.40-2.31 (m, 2H), 1.88-1.79 (m, 1H), 1.62-1.53 (m, 1H). MS m/z: [M+H]⁺ 477.25; HPLC>95%.

### Example 7: Synthesis of Compound 7

The synthetic route of (1R,5S,6r)-N-(2-(8-chloroimidazo[1,5-a]pyridin-3-yl)propan-2-yl)-3-azabicyclo[3.1.1]heptane-6-carboxamide Compound 7 is shown in Reaction Scheme 7.

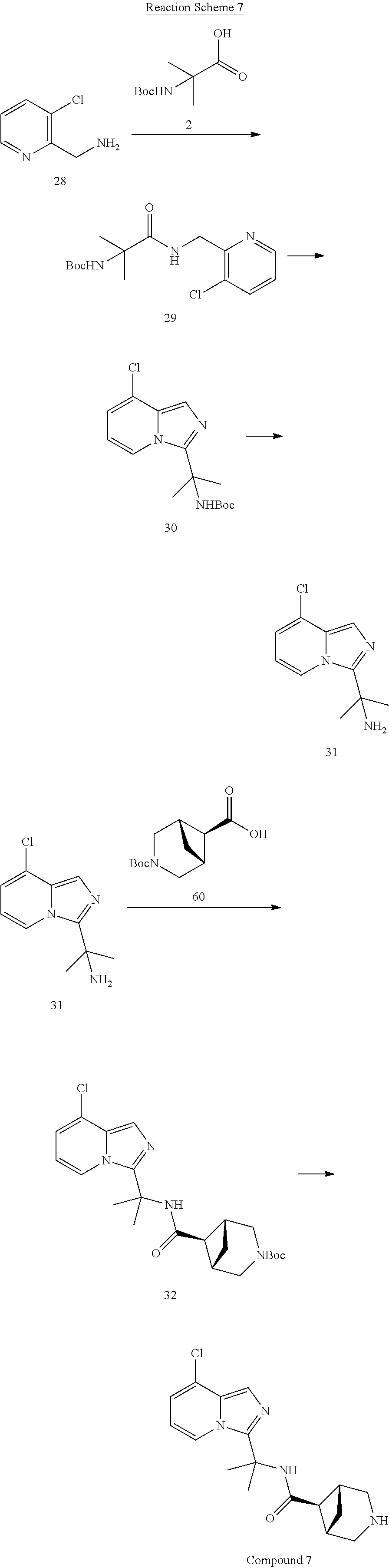

Step 1: Synthesis of tert-butyl (1-(((3-chloropyridin-2-yl)methyl)amino)-2-methyl-1-oxopropan-2-yl)carbamate 29.

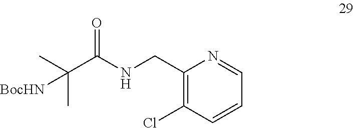 [SMILES: CC(C)(C)OC(=O)NC(C)(C)C(=O)NCc1ncccc1Cl]

3-chloropyridin-2-ylmethanamine 28 (2 g, 14.0 mmol) was added to a 100 mL round-bottom flask. 20 mL of DCM was added as the solvent. To the mixture, 2-(tert-butoxycarbonylamino)-2-methylpropanoic acid 2 (3.41 g, 16.8 mmol) and HATU (6.39 g, 16.8 mmol) were added. Finally, TEA (10 mL) was added and stirred at room temperature for 1 hour. LC-MS showed complete reaction of the starting material. The mixture was extracted with a DCM and NaHSO₄ solution. The organic layer was dried over anhydrous Na₂SO₄, and the solution was concentrated to obtain tert-butyl (1-(((3-chloropyridin-2-yl)methyl)amino)-2-methyl-1-oxopropan-2-yl)carbamate 29 (4.6 g, 100%). MS m/z: [M+H]⁺ 328.14.

Step 2: Synthesis of tert-butyl (2-(8-chloroimidazo[1,5-a]pyridin-3-yl)propan-2-yl)carbamate 30.

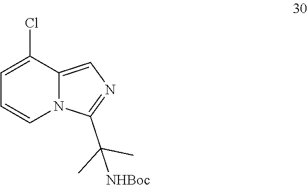 [SMILES: CC(C)(C)OC(=O)NC(C)(C)c1ncc2c(Cl)cccn12]

tert-butyl (1-(((3-chloropyridin-2-yl)methyl)amino)-2-methyl-1-oxopropan-2-yl)carbamate 29 (4.6 g, 14.0 mmol) was weighed, added to a 250 mL round-bottom flask, and dissolved in 60 mL of DCM. An appropriate amount of molecular sieves was added and stirred for 30 minutes. Then, Burgess reagent (5.0 g, 21.0 mmol) was added to the mixture and stirred for 2 hours. LC-MS showed complete reaction of the starting material. The mixture was filtered and the filtrate was directly purified by column chromatography to obtain the product tert-butyl (2-(8-chloroimidazo[1,5-a]pyridin-3-yl)propan-2-yl)carbamate (1.3 g, 30%) 30. MS m/z: [M+H]⁺ 310.13.

Step 3: Synthesis of 2-(8-chloroimidazo[1,5-a]pyridin-3-yl)propan-2-amine 31.

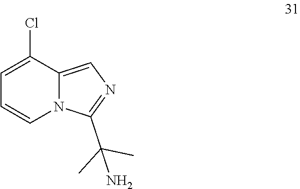

tert-butyl (2-(8-chloroimidazo[1,5-a]pyridin-3-yl)propan-2-yl)carbamate 30 (1.3 g, 4.2 mmol) was added to a 50 mL round-bottom flask. EtOH/HCl (5 mL) was added and stirred at room temperature for 30 minutes. The mixture was distilled under reduced pressure to obtain 2-(8-chloroimidazo[1,5-a]pyridin-3-yl)propan-2-amine 31 (793 mg, 90%). MS m/z: [M+H]⁺210.08.

Step 4: Synthesis of tert-butyl (1R,5S,6r)-6-((2-(8-chloroimidazo[1,5-a]pyridin-3-yl)propan-2-yl)carbamoyl)-3-azabicyclo[3.1.1]heptane-3-carboxylate 32.

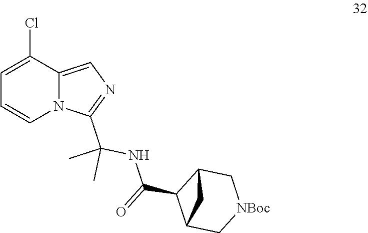 [SMILES: CC(C)(C)OC(=O)N1C[C@H]2C[C@@H](C1)[C@@H]2C(=O)NC(C)(C)c1ncc2c(Cl)cccn12]

2-(8-chloroimidazo[1,5-a]pyridin-3-yl)propan-2-amine 31 (793 mg, 3.8 mmol) was added to a 100 mL round-bottom flask. 20 mL of DCM was added as the solvent. To the mixture, (1R,5S,6r)-3-(tert-butoxycarbonyl)-3-azabicyclo[3.1.1]heptane-6-carboxylic acid 60 (1.0 g, 4.2 mmol) and HATU (1.7 g, 4.5 mmol) were added. Finally, TEA (5 mL) was added and stirred at room temperature for 1 hour. LC-MS showed complete reaction of the starting material. Extract the mixture with DCM and NaHSO₄ solution. The organic layer was dried over anhydrous Na₂SO₄, and the solution was concentrated. The product was purified by column chromatography to obtain tert-butyl (1R,5S,6r)-6-((2-(8-chloroimidazo[1,5-a]pyridin-3-yl)propan-2-yl)carbamoyl)-3-azabicyclo[3.1.1]heptane-3-carboxylate (1.3 g, 80%) 32. MS m/z: [M+H]⁺433.20.

Step 5: Synthesis of (1R,5S,6r)-N-(2-(8-chloroimidazo[1,5-a]pyridin-3-yl)propan-2-yl)-3-azabicyclo[3.1.1]heptane-6-carboxamide Compound 7.

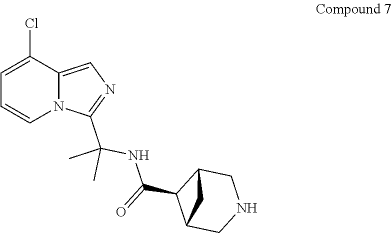 [SMILES: CC(C)(NC(=O)[C@H]1[C@@H]2CNC[C@H]1C2)c1ncc2c(Cl)cccn12]

tert-butyl (1R,5S,6r)-6-((2-(8-chloroimidazo[1,5-a]pyridin-3-yl)propan-2-yl)carbamoyl)-3-azabicyclo[3.1.1]heptane-3-carboxylate 32 (1.3 g, 3.0 mmol) was added to a 50 mL round-bottom flask. 10 mL of EtOH/HCl was added to the flask, stirred at room temperature for 30 minutes, and then distilled under reduced pressure to obtain (1R,5S,6r)-N-(2-(8-chloroimidazo[1,5-a]pyridin-3-yl)propan-2-yl)-3-azabicyclo[3.1.1]heptane-6-carboxamide Compound 7 (899 mg, 90%). ¹H NMR (500 MHz, Chloroform-d) δ 9.18 (dd, J=7.3, 1.3 Hz, 1H), 8.26 (s, 1H), 7.59 (dd, J=7.4, 1.5 Hz, 1H), 7.11 (t, J=7.3 Hz, 1H), 6.98 (s, 1H), 3.07-2.96 (m, 4H), 2.68 (t, J=7.4 Hz, 1H), 2.30-2.17 (m, 2H), 1.94-1.87 (m, 1H), 1.77-1.68 (m, 7H), 1.59-1.50 (m, 1H). MS m/z: [M+H]⁺ 333.15; HPLC>95%.

### Example 8: Synthesis of Compound 8

The synthetic route of (1R,5S,6r)-N-(2-(8-bromoimidazo[1,5-a]pyridin-3-yl)propan-2-yl)-3-azabicyclo[3.1.1]heptane-6-carboxamide Compound 8 is shown in Reaction Scheme 8.

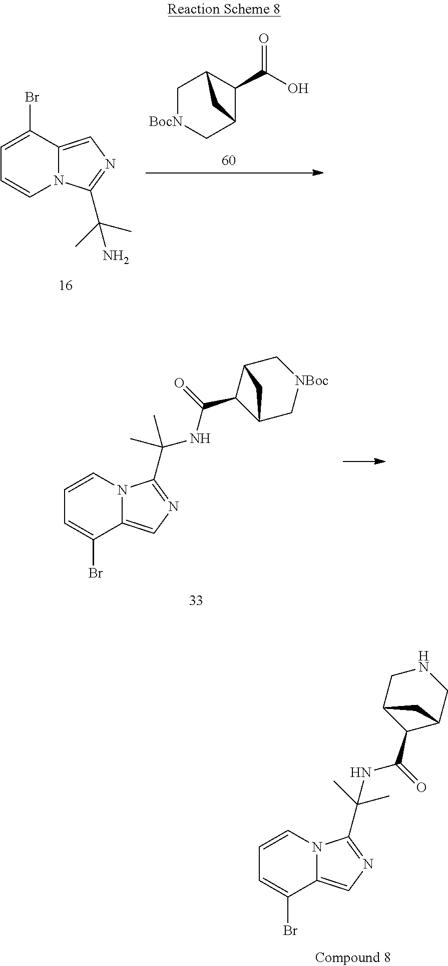 [SMILES: CC(C)(N)c1ncc2c(Br)cccn12 ; CC(C)(C)OC(=O)N1C[C@H]2C[C@@H](C1)[C@@H]2C(=O)O ; CC(C)(C)OC(=O)N1C[C@H]2C[C@@H](C1)[C@@H]2C(=O)NC(C)(C)c1ncc2c(Br)cccn12 ; CC(C)(NC(=O)[C@H]1[C@@H]2CNC[C@H]1C2)c1ncc2c(Br)cccn12]

Step 1: Synthesis of tert-butyl (1R,5S,6r)-6-((2-(8-bromoimidazo[1,5-a]pyridin-3-yl)propan-2-yl)carbamoyl)-3-azabicyclo[3.1.1]heptane-3-carboxylate 33

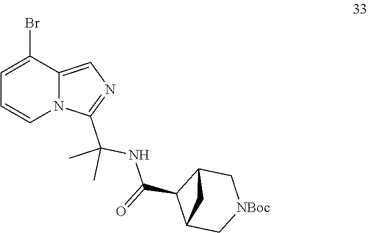 [SMILES: CC(C)(C)OC(=O)N1C[C@H]2C[C@@H](C1)[C@@H]2C(=O)NC(C)(C)c1ncc2c(Br)cccn12]

2-(8-bromoimidazo[1,5-a]pyridin-3-yl)propan-2-amine 16 (500 mg, 2 mmol) was added to a 100 mL round-bottom flask, and 10 mL of DCM was added as a solvent. To the mixture, (1R,5S,6r)-3-(tert-butoxycarbonyl)-3-azabicyclo[3.1.1]heptane-6-carboxylic acid 60 (531 mg, 2.2 mmol) and HATU (912 mg, 2.4 mmol) were added. Finally, TEA (5 mL) was added and stirred at room temperature for 1 hour. LC-MS showed the complete reaction of the starting material. The mixture was extracted with DCM and NaHSO₄ solution. The organic layer was dried over anhydrous Na₂SO₄ and concentrated. The product was purified by column chromatography to obtain tert-butyl (1R,5S,6r)-6-((2-(8-bromoimidazo[1,5-a]pyridin-3-yl)propan-2-yl)carbamoyl)-3-azabicyclo[3.1.1]heptane-3-carboxylate 33 (764 mg, 80%). MS m/z: [M+H]⁺ 477.15. (1R,5S,6r)-3-(tert-butoxycarbonyl)-3-azabicyclo[3.1.1]heptane-6-carboxylic acid

Step 2: Synthesis of (1R,5S,6r)-N-(2-(8-bromoimidazo[1,5-a]pyridin-3-yl)propan-2-yl)-3-azabicyclo[3.1.1]heptane-6-carboxamide Compound 8.

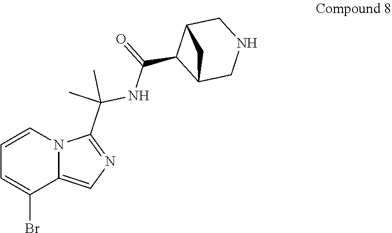 [SMILES: CC(C)(NC(=O)[C@H]1[C@@H]2CNC[C@H]1C2)c1ncc2c(Br)cccn12]

tert-butyl (1R,5S,6r)-6-((2-(8-bromoimidazo[1,5-a]pyridin-3-yl)propan-2-yl)carbamoyl)-3-azabicyclo[3.1.1]heptane-3-carboxylate 33 (764 mg, 1.6 mmol) was added to a 50 mL round-bottom flask followed by 5 mL of EtOH/HCl. The mixture was stirred at room temperature for 30 minutes, then distilled under reduced pressure to obtain (1R,5S,6r)-N-(2-(8-bromoimidazo[1,5-a]pyridin-3-yl)propan-2-yl)-3-azabicyclo[3.1.1]heptane-6-carboxamide Compound 8 (543 mg, 90%). ¹H NMR (500 MHz, Chloroform-d) δ 9.25 (dd, J=7.3, 1.3 Hz, 1H), 8.11 (s, 1H), 7.69 (dd, J=7.4, 1.5 Hz, 1H), 7.14 (t, J=7.3 Hz, 1H), 6.98 (s, 1H), 3.07-2.96 (m, 4H), 2.68 (t, J=7.4 Hz, 1H), 2.30-2.17 (m, 2H), 1.94-1.87 (m, 1H), 1.77-1.68 (m, 7H), 1.59-1.50 (m, 1H). MS m/z: [M+H]⁺377.10; HPLC>95%.

### Example 9: Synthesis of Compound 9

The synthetic route of (1R,5S,6r)-N-(2-(1-cyclopropylimidazo[1,5-a]pyridin-3-yl)propan-2-yl)-3-azabicyclo[3.1.1]heptane-6-carboxamide Compound 9 is shown in Reaction Scheme 9.

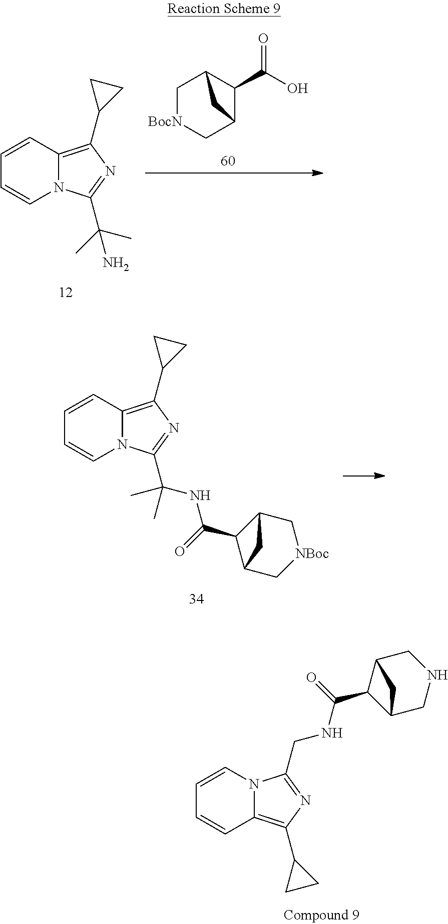 [SMILES: CC(C)(N)c1nc(C2CC2)c2ccccn12 ; CC(C)(C)OC(=O)N1C[C@H]2C[C@@H](C1)[C@@H]2C(=O)O ; CC(C)(C)OC(=O)N1C[C@H]2C[C@@H](C1)[C@@H]2C(=O)NC(C)(C)c1nc(C2CC2)c2ccccn12 ; O=C(NCc1nc(C2CC2)c2ccccn12)[C@H]1[C@@H]2CNC[C@H]1C2]

Step 1: Synthesis of tert-butyl (1R,5S,6r)-6-((2-(1-cyclopropylimidazo[1,5-a]pyridin-3-yl)propan-2-yl)carbamoyl)-3-azabicyclo[3.1.1]heptane-3-carboxylate 34

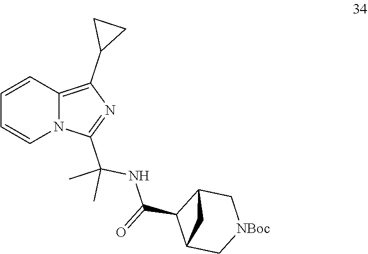 [SMILES: CC(C)(C)OC(=O)N1C[C@H]2C[C@@H](C1)[C@@H]2C(=O)NC(C)(C)c1nc(C2CC2)c2ccccn12]

2-(1-cyclopropylimidazo[1,5-a]pyridin-3-yl)propan-2-amine 21 (500 mg, 2.3 mmol) was added to a 100 mL round-bottom flask followed the addition of 10 ml of DCM as a solvent. To the mixture, (1R,5S,6r)-3-(tert-butoxycarbonyl)-3-azabicyclo[3.1.1]heptane-6-carboxylic acid 60 (609 mg, 2.5 mmol) and HATU (1.0 g, 2.8 mmol) were added. Finally, TEA (5 mL) was added and stirred at room temperature for 1 hour. LC-MS showed complete reaction of the starting material. Extract the mixture with DCM and NaHSO₄ solution. The organic layer was dried over anhydrous Na₂SO₄, and the organic layer was concentrated. The product was purified by column chromatography to obtain tert-butyl (1R,5S,6r)-6-((2-(1-cyclopropylimidazo[1,5-a]pyridin-3-yl)propan-2-yl)carbamoyl)-3-azabicyclo[3.1.1]heptane-3-carboxylate 34 (806 mg, 80%). MS m/z: [M+H]⁺439.27.

Step 2: Synthesis of (1R,5S,6r)-N-(2-(1-cyclopropylimidazo[1,5-a]pyridin-3-yl)propan-2-yl)-3-azabicyclo[3.1.1]heptane-6-carboxamide Compound 9.

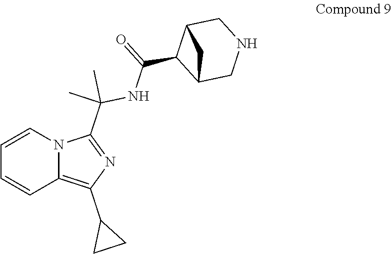 [SMILES: CC(C)(NC(=O)[C@H]1[C@@H]2CNC[C@H]1C2)c1nc(C2CC2)c2ccccn12]

tert-butyl (1R,5S,6r)-6-((2-(1-cyclopropylimidazo[1,5-a]pyridin-3-yl)propan-2-yl)carbamoyl)-3-azabicyclo[3.1.1]heptane-3-carboxylate 34 (806 mg, 1.8 mmol) was added to a 50 mL round-bottom flask followed by EtOH/HCl (5 mL). The mixture was stirred at room temperature for 30 minutes and then vacuum distillation was performed to obtain (1R,5S,6r)-N-(2-(1-cyclopropylimidazo[1,5-a]pyridin-3-yl)propan-2-yl)-3-azabicyclo[3.1.1]heptane-6-carboxamide Compound 9 (548 mg, 90%). ¹H NMR (500 MHz, Chloroform-d) δ 8.82 (dd, J=7.2, 1.3 Hz, 1H), 7.63 (dd, J=9.6, 1.5 Hz, 1H), 7.22-7.15 (m, 1H), 7.05-6.96 (m, 2H), 3.07-2.96 (m, 4H), 2.83-2.76 (m, 1H), 2.68 (t, J=7.4 Hz, 1H), 2.30-2.17 (m, 2H), 1.94-1.87 (m, 1H), 1.77-1.68 (m, 7H), 1.59-1.50 (m, 1H), 1.08-0.94 (m, 4H). MS m/z: [M+H]⁺339.22; HPLC>95%.

### Example 10: Synthesis of Compound 10

The synthetic route of (1R,5S,6r)-N-(2-(8-methoxyimidazo[1,5-a]pyridin-3-yl)propan-2-yl)-3-azabicyclo[3.1.1]heptane-6-carboxamide Compound 10 is shown in Reaction Scheme 10.

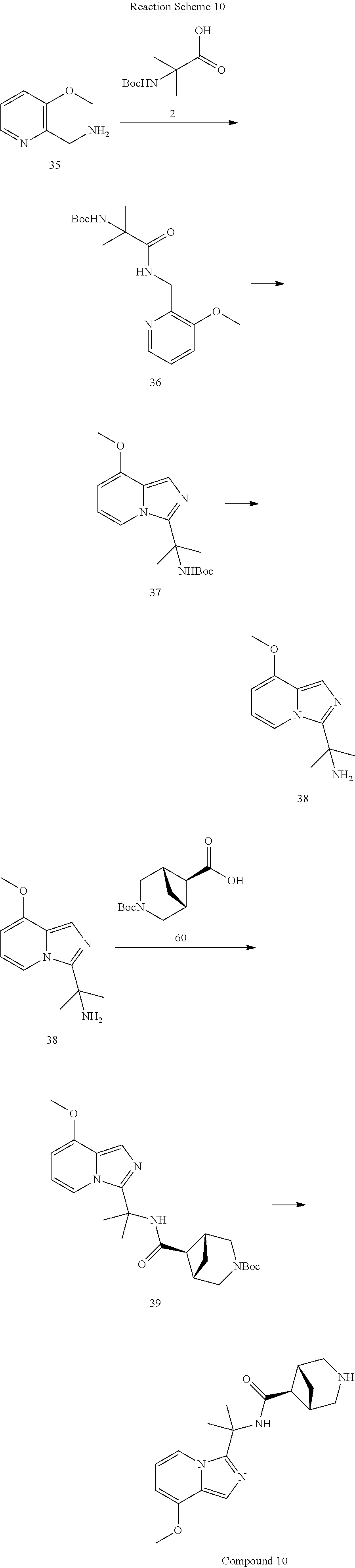

Step 1: Synthesis of tert-butyl (1-(((3-methoxypyridin-2-yl)methyl)amino)-2-methyl-1-oxopropan-2-yl)carbamate 36.

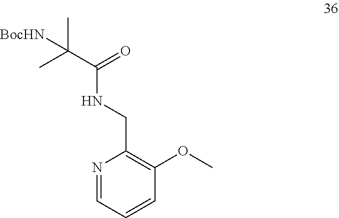 [SMILES: COc1cccnc1CNC(=O)C(C)(C)NC(=O)OC(C)(C)C]

(3-methoxypyridin-2-yl)methanamine 35 (2 g, 14.5 mmol) was added to a 100 mL round-bottom flask followed by 20 mL DCM as a solvent. To the mixture, 2-(tert-butoxycarbonyl)amino-2-methylpropanoic acid 2 (3.54 g, 17.4 mmol) and HATU (6.62 g, 17.4 mmol) were added. Finally, TEA (10 mL) was added and stirred at room temperature for 1 hour. LC-MS showed complete reaction of the starting material. The mixture was extracted with DCM and NaHSO₄ solution. The organic layer was dried over anhydrous Na₂SO₄ and the solution was concentrated to obtain tert-butyl (1-(((3-methoxypyridin-2-yl)methyl)amino)-2-methyl-1-oxopropan-2-yl)carbamate 36 (4.7 g, 100%). MS m/z: [M+H]⁺ 324.19.

Step 2: Synthesis of tert-butyl (2-(8-methoxyimidazo[1,5-a]pyridin-3-yl)propan-2-yl)carbamate 37.

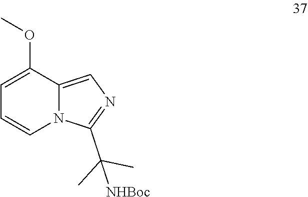 [SMILES: COc1cccn2c(C(C)(C)NC(=O)OC(C)(C)C)ncc12]

tert-butyl (1-(((3-methoxypyridin-2-yl)methyl)amino)-2-methyl-1-oxopropan-2-yl)carbamate 36 (4.7 g, 14.5 mmol) was weighed into a 250 mL round-bottom flask and dissolved in 60 mL DCM. An appropriate amount of molecular sieves were added and stirred for 30 minutes. Then, Burgess reagent (5.2 g, 21.8 mmol) was added to the mixture and stirred for 2 hours. LC-MS analysis showed complete reaction of the starting material. The mixture was filtered and the filtrate was directly concentrated. The product was purified by column chromatography to obtain tert-butyl (2-(8-methoxyimidazo[1,5-a]pyridin-3-yl)propan-2-yl)carbamate 37 (1.3 g, 30%). MS m/z: [M+H]⁺ 306.18.

Step 3: Synthesis of 2-(8-methoxyimidazo[1,5-a]pyridin-3-yl)propan-2-amine 38

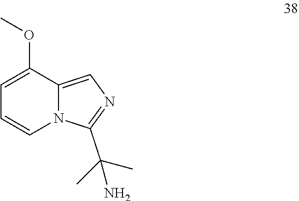

tert-butyl (2-(8-methoxyimidazo[1,5-a]pyridin-3-yl)propan-2-yl)carbamate 37 (1.3 g, 4.4 mmol) was added to a 50 mL round-bottom flask followed by EtOH/HCl (5 mL). The mixture was stirred at room temperature for 30 minutes, and then vacuum distillation was performed to obtain 2-(8-methoxyimidazo[1,5-a]pyridin-3-yl)propan-2-amine 38 (813 mg, 90%). MS m/z: [M+H]⁺206.13.

Step 4: Synthesis of tert-butyl (1R,5S,6r)-6-((2-(8-methoxyimidazo[1,5-a]pyridin-3-yl)propan-2-yl)carbamoyl)-3-azabicyclo[3.1.1]heptane-3-carboxylate 39

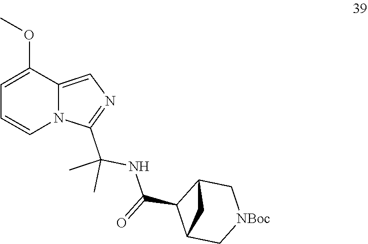 [SMILES: COc1cccn2c(C(C)(C)NC(=O)[C@@H]3[C@@H]4C[C@H]3CN(C(=O)OC(C)(C)C)C4)ncc12]

2-(8-methoxyimidazo[1,5-a]pyridin-3-yl)propan-2-amine 38 (813 mg, 4.0 mmol) was added to a 100 mL round-bottom flask followed by 20 mL DCM as a solvent. To the mixture, (1R,5S,6r)-3-(tert-butoxycarbonyl)-3-azabicyclo[3.1.1]heptane-6-carboxylic acid 6 (1.1 g, 4.4 mmol) and HATU (1.8 g, 4.8 mmol) were added. Finally, TEA (5 mL) was added and stirred at room temperature for 1 hour. LC-MS showed complete reaction of the starting material. Extract the mixture with DCM and NaHSO₄ solution. The organic layer was dried over anhydrous Na₂SO₄, and the organic layer was concentrated. The product was purified by column chromatography to obtain tert-butyl (1R,5S,6r)-6-((2-(8-methoxyimidazo[1,5-a]pyridin-3-yl)propan-2-yl)carbamoyl)-3-azabicyclo[3.1.1]heptane-3-carboxylate 39 (1.4 g, 80%). MS m/z: [M+H]⁺ 429.25.

Step 5: Synthesis of (1R,5S,6r)-N-(2-(8-methoxyimidazo[1,5-a]pyridin-3-yl)propan-2-yl)-3-azabicyclo[3.1.1]heptane-6-carboxamide Compound 10

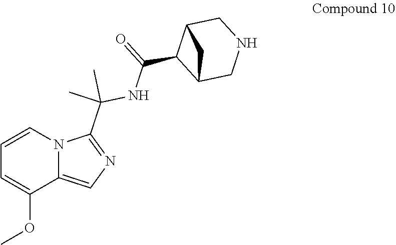 [SMILES: COc1cccn2c(C(C)(C)NC(=O)[C@H]3[C@@H]4CNC[C@H]3C4)ncc12]

tert-butyl (1R,5S,6r)-6-((2-(8-methoxyimidazo[1,5-a]pyridin-3-yl)propan-2-yl)carbamoyl)-3-azabicyclo[3.1.1]heptane-3-carboxylate 39 (1.4 g, 3.2 mmol) was added to a 50 mL round-bottom flask followed by EtOH/HCl (10 mL). The mixture was stirred at room temperature for 30 minutes. Vacuum distillation was performed to obtain (1R,5S,6r)-N-(2-(8-methoxyimidazo[1,5-a]pyridin-3-yl)propan-2-yl)-3-azabicyclo[3.1.1]heptane-6-carboxamide Compound 10 (946 mg, 90%). ¹H NMR (500 MHz, Chloroform-d) δ 8.72 (dd, J=7.2, 1.4 Hz, 1H), 8.42 (s, 1H), 7.10 (t, J=7.3 Hz, 1H), 7.04-6.97 (m, 2H), 3.91 (s, 3H), 3.07-2.96 (m, 4H), 2.68 (t, J=7.4 Hz, 1H), 2.30-2.17 (m, 2H), 1.94-1.87 (m, 1H), 1.73 (dd, J=12.4, 6.7 Hz, 7H), 1.59-1.50 (m, 1H). MS m/z: [M+H]⁺329.20; HPLC>95%.

### Example 11: Synthesis of Compound 11

The synthetic route of (1R,5S,6r)-N-(2-(8-(trimethylsilyl)imidazo[1,5-a]pyridin-3-yl)propan-2-yl)-3-azabicyclo[3.1.1]heptane-6-carboxamide Compound 11 is shown in Reaction Scheme 11.

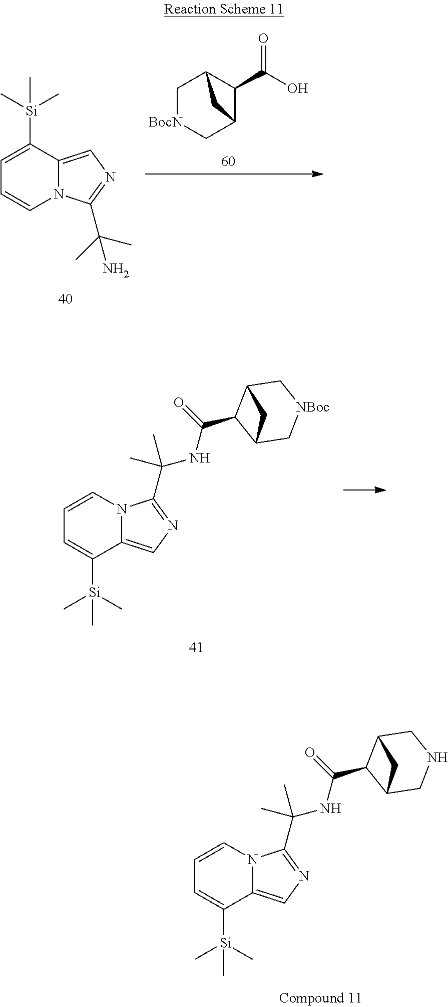 [SMILES: CC(C)(N)c1ncc2c([Si](C)(C)C)cccn12 ; CC(C)(C)OC(=O)N1C[C@H]2C[C@@H](C1)[C@@H]2C(=O)O ; CC(C)(C)OC(=O)N1C[C@H]2C[C@@H](C1)[C@@H]2C(=O)NC(C)(C)c1ncc2c([Si](C)(C)C)cccn12 ; CC(C)(NC(=O)[C@H]1[C@@H]2CNC[C@H]1C2)c1ncc2c([Si](C)(C)C)cccn12]

Step 1: Synthesis of tert-butyl (1R,5S,6r)-6-((2-(8-(trimethylsilyl)imidazo[1,5-a]pyridin-3-yl)propan-2-yl)carbamoyl)-3-azabicyclo[3.1.1]heptane-3-carboxylate 41

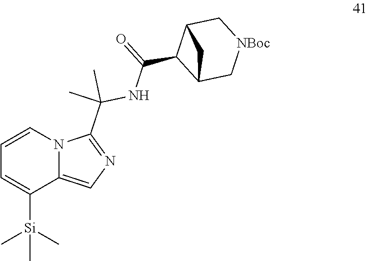 [SMILES: CC(C)(C)OC(=O)N1C[C@H]2C[C@@H](C1)[C@@H]2C(=O)NC(C)(C)c1ncc2c([Si](C)(C)C)cccn12]

2-(8-(trimethylsilyl)imidazo[1,5-a]pyridin-3-yl)propan-2-amine 40 (500 mg, 2.0 mmol) was added to a 100 mL round-bottom flask followed by 10 mL DCM as a solvent. To the mixture, (1R,5S,6r)-3-(tert-butoxycarbonyl)-3-azabicyclo[3.1.1]heptane-6-carboxylic acid 60 (530 mg, 2.2 mmol) and HATU (912.55 mg, 2.4 mmol) were added. Finally, TEA (5 mL) was added and stirred at room temperature for 1 hour. LC-MS showed complete reaction of the starting material. The mixture was extracted with DCM and NaHSO₄ solution. The organic layer was dried over anhydrous Na₂SO₄, and the organic layer was concentrated. The product was purified by column chromatography to obtain tert-butyl (1R,5S,6r)-6-((2-(8-(trimethylsilyl)imidazo[1,5-a]pyridin-3-yl)propan-2-yl)carbamoyl)-3-azabicyclo[3.1.1]heptane-3-carboxylate 41 (753 mg, 80%). MS m/z: [M+H]⁺471.27.

Step 2: Synthesis of (1R,5S,6r)-N-(2-(8-(trimethylsilyl)imidazo[1,5-a]pyridin-3-yl)propan-2-yl)-3-azabicyclo[3.1.1]heptane-6-carboxamide Compound 11

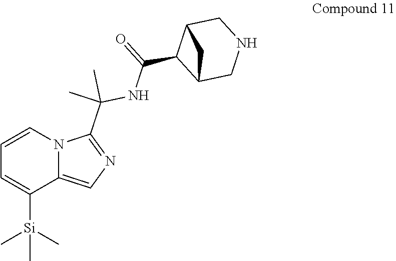 [SMILES: CC(C)(NC(=O)[C@H]1[C@@H]2CNC[C@H]1C2)c1ncc2c([Si](C)(C)C)cccn12]

tert-butyl (1R,5S,6r)-6-((2-(8-(trimethylsilyl)imidazo[1,5-a]pyridin-3-yl)propan-2-yl)carbamoyl)-3-azabicyclo[3.1.1]heptane-3-carboxylate 41 (753 mg, 1.6 mmol) was added to a 50 mL round-bottom flask followed by EtOH/HCl (5 mL). The mixture was stirred at room temperature for 30 minutes. Vacuum distillation was performed to obtain (1R,5S,6r)-N-(2-(8-(trimethylsilyl)imidazo[1,5-a]pyridin-3-yl)propan-2-yl)-3-azabicyclo[3.1.1]heptane-6-carboxamide Compound 11 (533 mg, 90%). 1H NMR (500 MHZ, Chloroform-d) δ 9.07 (dd, J=7.1, 1.3 Hz, 1H), 7.95 (s, 1H), 7.51 (dd, J=8.3, 1.5 Hz, 1H), 7.01 (s, 1H), 6.91 (dd, J=8.2, 7.1 Hz, 1H), 3.06-2.96 (m, 4H), 2.64 (t, J=7.0 Hz, 1H), 2.32-2.20 (m, 2H), 1.94-1.87 (m, 1H), 1.78-1.69 (m, 1H), 1.58-1.49 (m, 1H), 0.12 (s, 7H). MS m/z: [M+H]⁺371.23; HPLC>95%.

### Example 12: Synthesis of Compound 12

The synthetic route of (1R,5S,6r)-N-(2-(8-(4-methoxyphenyl)imidazo[1,5-a]pyridin-3-yl)propan-2-yl)-3-azabicyclo[3.1.1]heptane-6-carboxamide Compound 12 is shown in Reaction Scheme 12.

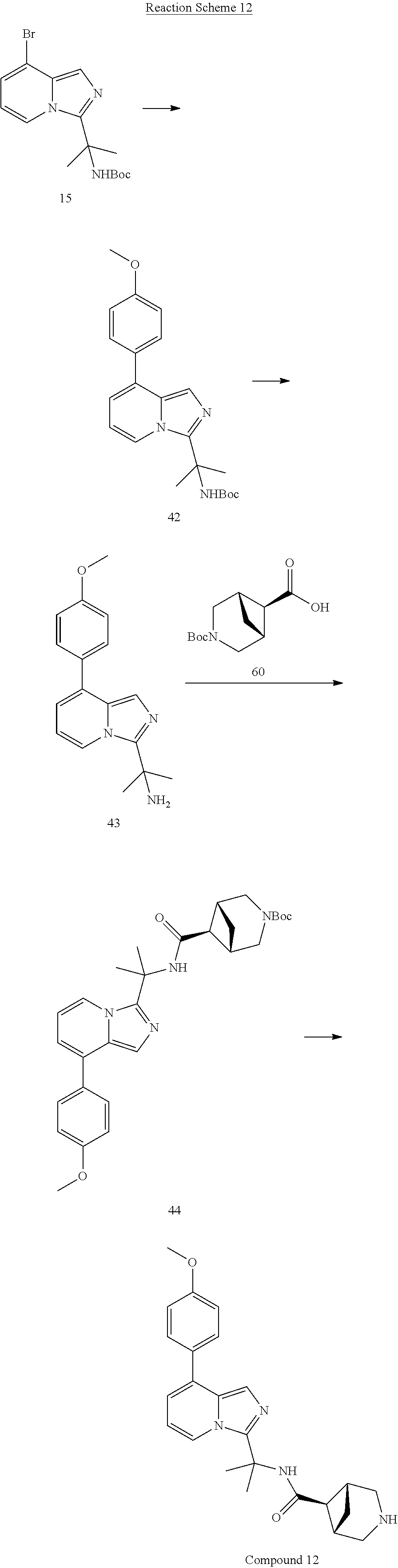 [SMILES: CC(C)(C)OC(=O)NC(C)(C)c1ncc2c(Br)cccn12 ; COc1ccc(-c2cccn3c(C(C)(C)NC(=O)OC(C)(C)C)ncc23)cc1 ; COc1ccc(-c2cccn3c(C(C)(C)N)ncc23)cc1 ; CC(C)(C)OC(=O)N1C[C@H]2C[C@@H](C1)[C@@H]2C(=O)O ; COc1ccc(-c2cccn3c(C(C)(C)NC(=O)[C@@H]4[C@@H]5C[C@H]4CN(C(=O)OC(C)(C)C)C5)ncc23)cc1 ; COc1ccc(-c2cccn3c(C(C)(C)NC(=O)[C@H]4[C@@H]5CNC[C@H]4C5)ncc23)cc1]

Step 1: Synthesis of tert-butyl (2-(8-(4-methoxyphenyl)imidazo[1,5-a]pyridin-3-yl)propan-2-yl)carbamate 42.

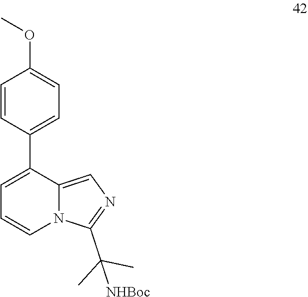 [SMILES: COc1ccc(-c2cccn3c(C(C)(C)NC(=O)OC(C)(C)C)ncc23)cc1]

tert-butyl (2-(8-bromoimidazo[1,5-a]pyridin-3-yl)propan-2-yl)carbamate 15 (1.0 g, 2.8 mmol) was added to a 100 mL round-bottom flask followed by 10 mL of DMF as a solvent. bis(triphenylphosphine)palladium(II) dichloride (211 mg, 0.3 mmol) and 4-methoxyphenylboronic acid (511 mg, 3.4 mmol) were added to the mixture. The mixture was heated to 80° C. and stirred for 2 hours. LC-MS showed complete reaction of the starting material. The mixture was extracted with water and ethyl acetate (EA). The organic layer was dried over anhydrous Na₂SO₄, concentrated, and purified by column chromatography to obtain tert-butyl (2-(8-(4-methoxyphenyl)imidazo[1,5-a]pyridin-3-yl)propan-2-yl)carbamate 42 (748 mg, 70%). MS m/z: [M+H]⁺ 382.21.

Step 2: Synthesis of 2-(8-(4-methoxyphenyl)imidazo[1,5-a]pyridin-3-yl)propan-2-amine 43

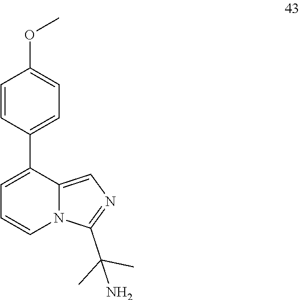 [SMILES: COc1ccc(-c2cccn3c(C(C)(C)N)ncc23)cc1]

tert-butyl (2-(8-(4-methoxyphenyl)imidazo[1,5-a]pyridin-3-yl)propan-2-yl)carbamate 42 (748 mg, 2.0 mmol) was added to a 50 mL round-bottom flask followed by EtOH/HCl (5 mL). The mixture was stirred at room temperature for 30 minutes, then vacuum distillation was performed to obtain 2-(8-(4-methoxyphenyl)imidazo[1,5-a]pyridin-3-yl)propan-2-amine (673 mg, 90%) 43. MS m/z: [M+H]⁺ 282.16.

Step 3: Synthesis of tert-butyl (1R,5S,6r)-6-((2-(8-(4-methoxyphenyl)imidazo[1,5-a]pyridin-3-yl)propan-2-yl)carbamoyl)-3-azabicyclo[3.1.1]heptane-3-carboxylate 44

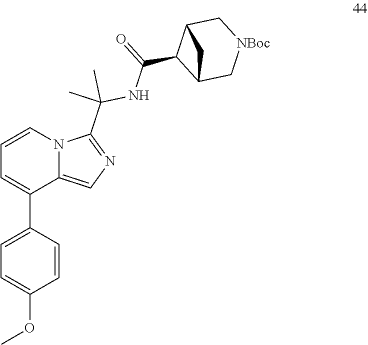 [SMILES: COc1ccc(-c2cccn3c(C(C)(C)NC(=O)[C@@H]4[C@@H]5C[C@H]4CN(C(=O)OC(C)(C)C)C5)ncc23)cc1]

2-(8-(4-methoxyphenyl)imidazo[1,5-a]pyridin-3-yl)propan-2-amine 43 (673 mg, 1.8 mmol) was added to a 100 mL round-bottom flask followed by 10 mL DCM as the solvent. To the mixture, (1R,5S,6r)-3-(tert-butoxycarbonyl)-3-azabicyclo[3.1.1]heptane-6-carboxylic acid 60 (472 mg, 2.0 mmol) and HATU (821 mg, 2.2 mmol) were added. Finally, TEA (5 mL) was added and stirred at room temperature for 1 hour. LC-MS showed complete reaction of the starting material. The mixture was extracted with a DCM and NaHSO₄ solution. The organic layer was dried over anhydrous Na₂SO₄, and the organic layer was concentrated. The product was purified by column chromatography to obtain tert-butyl (1R,5S,6r)-6-((2-(8-(4-methoxyphenyl)imidazo[1,5-a]pyridin-3-yl)propan-2-yl)carbamoyl)-3-azabicyclo[3.1.1]heptane-3-carboxylate 44 (727 mg, 80%). MS m/z: [M+H]⁺ 505.28.

Step 4: Synthesis of (1R,5S,6r)-N-(2-(8-(4-methoxyphenyl)imidazo[1,5-a]pyridin-3-yl)propan-2-yl)-3-azabicyclo[3.1.1]heptane-6-carboxamide Compound 12

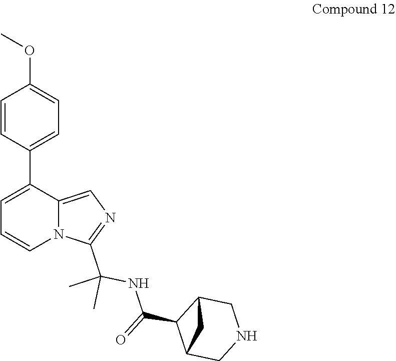 [SMILES: COc1ccc(-c2cccn3c(C(C)(C)NC(=O)[C@H]4[C@@H]5CNC[C@H]4C5)ncc23)cc1]

Add tert-butyl (1R,5S,6r)-6-((2-(8-(4-methoxyphenyl)imidazo[1,5-a]pyridin-3-yl)propan-2-yl)carbamoyl)-3-azabicyclo[3.1.1]heptane-3-carboxylate 44 (727 mg, 1.4 mmol) to a 50 mL round-bottom flask. Add 5 mL EtOH/HCl and stir at room temperature for 30 minutes. Perform vacuum distillation to obtain (1R,5S,6r)-N-(2-(8-(4-methoxyphenyl)imidazo[1,5-a]pyridin-3-yl)propan-2-yl)-3-azabicyclo[3.1.1]heptane-6-carboxamide Compound 12 (524 mg, 90%). ¹H NMR (500 MHZ, Chloroform-d) δ 9.18 (dd, J=7.2, 1.4 Hz, 1H), 8.21 (s, 1H), 7.86 (dd, J=6.9, 1.5 Hz, 1H), 7.46-7.40 (m, 2H), 7.10 (t, J=7.1 Hz, 1H), 7.00-6.94 (m, 2H), 6.94 (s, 1H), 3.82 (s, 3H), 3.06-2.96 (m, 4H), 2.64 (t, J=7.0 Hz, 1H), 2.32-2.20 (m, 2H), 1.94-1.87 (m, 1H), 1.78-1.69 (m, 1H), 1.58-1.49 (m, 1H). MS m/z: [M+H]⁺405.23; HPLC>95%.

### Example 13: Synthesis of Compound 13

The synthetic route of (1R,5S,6r)-N-(2-(8-(methylsulfinyl)imidazo[1,5-a]pyridin-3-yl)propan-2-yl)-3-azabicyclo[3.1.1]heptane-6-carboxamide Compound 13 is shown in Reaction Scheme 13.

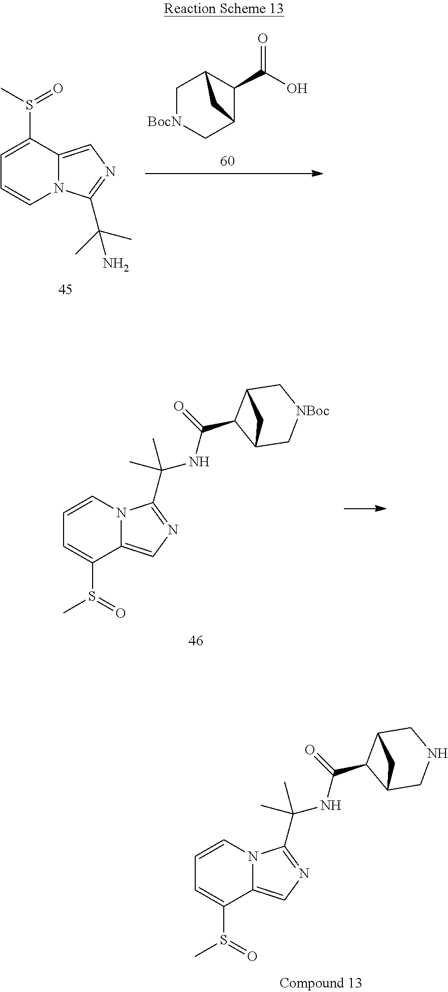 [SMILES: CS(=O)c1cccn2c(C(C)(C)N)ncc12 ; CC(C)(C)OC(=O)N1C[C@H]2C[C@@H](C1)[C@@H]2C(=O)O ; CS(=O)c1cccn2c(C(C)(C)NC(=O)[C@@H]3[C@@H]4C[C@H]3CN(C(=O)OC(C)(C)C)C4)ncc12 ; CS(=O)c1cccn2c(C(C)(C)NC(=O)[C@H]3[C@@H]4CNC[C@H]3C4)ncc12]

Step 1: Synthesis of tert-butyl (1R,5S,6r)-6-((2-(8-(methylsulfinyl)imidazo[1,5-a]pyridin-3-yl)propan-2-yl)carbamoyl)-3-azabicyclo[3.1.1]heptane-3-carboxylate 46

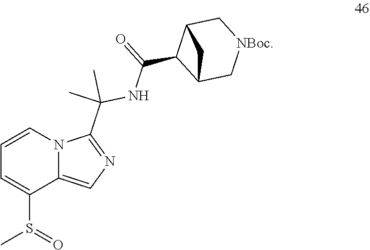 [SMILES: CS(=O)c1cccn2c(C(C)(C)NC(=O)[C@@H]3[C@@H]4C[C@H]3CN(C(=O)OC(C)(C)C)C4)ncc12]

2-(8-(methylsulfinyl)imidazo[1,5-a]pyridin-3-yl)propan-2-amine 45 (500 mg, 2.1 mmol) was added to a 100 mL round-bottom flask followed by 20 mL DCM as a solvent. To the mixture, (1R,5S,6r)-3-(tert-butoxycarbonyl)-3-azabicyclo[3.1.1]heptane-6-carboxylic acid 60 (554 mg, 2.3 mmol) and HATU (958 mg, 2.5 mmol) were added. Finally, TEA (5 mL) was added and stirred at room temperature for 1 hour. LC-MS showed complete reaction of the starting material. The mixture was extracted using a DCM and NaHSO₄ solution. The organic layer was dried over anhydrous Na₂SO₄ and the organic layer was concentrated. The product was purified by column chromatography to obtain tert-butyl (1R,5S,6r)-6-((2-(8-(methylsulfinyl)imidazo[1,5-a]pyridin-3-yl)propan-2-yl)carbamoyl)-3-azabicyclo[3.1.1]heptane-3-carboxylate 46 (774 mg, 80%). MS m/z: [M+H]⁺461.22.

Step 2: Synthesis of (1R,5S,6r)-N-(2-(8-(methylsulfinyl)imidazo[1,5-a]pyridin-3-yl)propan-2-yl)-3-azabicyclo[3.1.1]heptane-6-carboxamide Compound 13

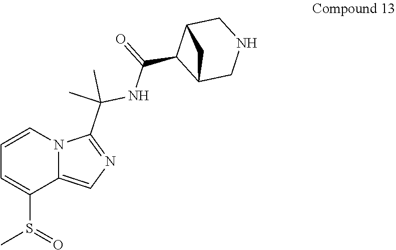 [SMILES: CS(=O)c1cccn2c(C(C)(C)NC(=O)[C@H]3[C@@H]4CNC[C@H]3C4)ncc12]

tert-butyl (1R,5S,6r)-6-((2-(8-(methylsulfinyl)imidazo[1,5-a]pyridin-3-yl)propan-2-yl)carbamoyl)-3-azabicyclo[3.1.1]heptane-3-carboxylate 46 (774 mg, 1.7 mmol) was added to a 50 mL round-bottom flask followed by EtOH/HCl (10 mL). The mixture was stirred at room temperature for 30 minutes, then vacuum distillation was performed to obtain (1R,5S,6r)-N-(2-(8-(methylsulfinyl)imidazo[1,5-a]pyridin-3-yl)propan-2-yl)-3-azabicyclo[3.1.1]heptane-6-carboxamide Compound 13 (552 mg, 90%). ¹H NMR (500 MHZ, Chloroform-d) δ 9.24 (dd, J=6.6, 1.3 Hz, 1H), 8.02-7.95 (m, 2H), 7.20 (t, J=6.8 Hz, 1H), 6.98 (s, 1H), 3.06-2.96 (m, 4H), 2.86 (s, 2H), 2.64 (t, J=7.0 Hz, 1H), 2.32-2.20 (m, 2H), 1.94-1.87 (m, 1H), 1.78-1.69 (m, 1H), 1.58-1.49 (m, 1H). MS m/z: [M+H]⁺361,17; HPLC>95%.

### Example 14: Synthesis of Compound 16

The synthetic route of (1R,5S,6r)-N-((8-bromoimidazo[1,5-a]pyridin-3-yl)methyl)-3-azabicyclo[3.1.1]heptane-6-carboxamide Compound 16 is shown in Reaction Scheme 14.

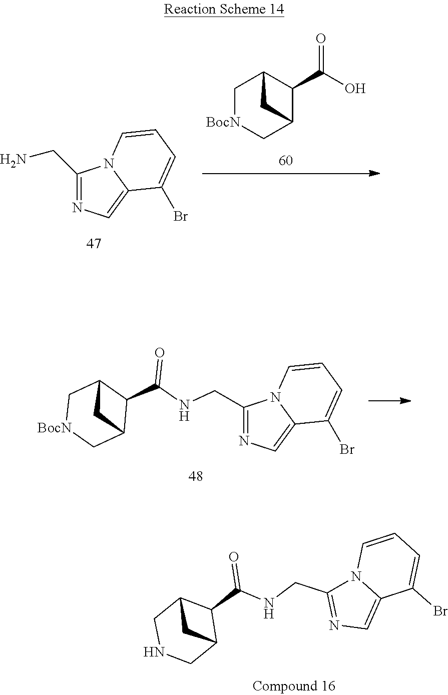 [SMILES: NCc1ncc2c(Br)cccn12 ; CC(C)(C)OC(=O)N1C[C@H]2C[C@@H](C1)[C@@H]2C(=O)O ; CC(C)(C)OC(=O)N1C[C@H]2C[C@@H](C1)[C@@H]2C(=O)NCc1ncc2c(Br)cccn12 ; O=C(NCc1ncc2c(Br)cccn12)[C@H]1[C@@H]2CNC[C@H]1C2]

Step 1: Synthesis of tert-butyl (1R,5S,6r)-6-(((8-bromoimidazo[1,5-a]pyridin-3-yl)methyl)carbamoyl)-3-azabicyclo[3.1.1]heptane-3-carboxylate 48.

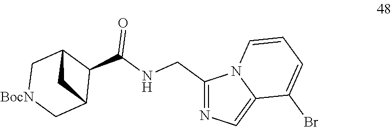 [SMILES: CC(C)(C)OC(=O)N1C[C@H]2C[C@@H](C1)[C@@H]2C(=O)NCc1ncc2c(Br)cccn12]

8-bromoimidazo[1,5-a]pyridin-3-ylmethanamine 47 (500 mg, 2.2 mmol) was added to a 100 mL round-bottom flask followed by the addition of 10 mL of DCM as a solvent. To the mixture, (1R,5S,6r)-3-tert-butoxycarbonyl-3-azabicyclo[3.1.1]heptane-6-carboxylic acid (578 mg, 2.4 mmol) 60 and HATU (989 mg, 2.6 mmol) were added. Finally, TEA (5 mL) was added and stirred at room temperature for 1 hour. LC-MS showed complete reaction of the starting material. The mixture was extracted with a DCM and NaHSO₄ solution. The organic layer was dried over anhydrous Na₂SO₄ and the organic layer was concentrated. The product was purified by column chromatography to obtain tert-butyl (1R,5S,6r)-6-(((8-bromoimidazo[1,5-a]pyridin-3-yl)methyl)carbamoyl)-3-azabicyclo[3.1.1]heptane-3-carboxylate 48 (791 mg, 80%). MS m/z: [M+H]⁺ 449.12.

Step 2: Synthesis of (1R,5S,6r)-N-((8-bromoimidazo[1,5-a]pyridin-3-yl)methyl)-3-azabicyclo[3.1.1]heptane-6-carboxamide Compound 16

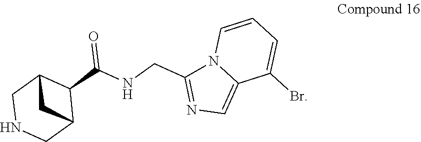 [SMILES: O=C(NCc1ncc2c(Br)cccn12)[C@H]1[C@@H]2CNC[C@H]1C2]

tert-butyl (1R,5S,6r)-6-(((8-bromoimidazo[1,5-a]pyridin-3-yl)methyl)carbamoyl)-3-azabicyclo[3.1.1]heptane-3-carboxylate 48 (791 mg, 1.8 mmol) was added to a 50 mL round-bottom flask followed by the addition of EtOH/HCl (5 mL) and the mixture was stirred at room temperature for 30 minutes. Vacuum distillation was performed to obtain (1R,5S,6r)-N-((8-bromoimidazo[1,5-a]pyridin-3-yl)methyl)-3-azabicyclo[3.1.1]heptane-6-carboxamide Compound 16 (566 mg, 90%). ¹H NMR (500 MHz, Chloroform-d) δ 9.25 (dd, J=8.1, 1.3 Hz, 1H), 7.70 (dd, J=7.6, 1.5 Hz, 1H), 7.63 (s, 1H), 7.50 (t, J=4.1 Hz, 1H), 7.08 (dd, J=8.1, 7.3 Hz, 1H), 4.60 (d, J=4.1 Hz, 2H), 3.06-2.96 (m, 4H), 2.52 (t, J=7.6 Hz, 1H), 2.31-2.21 (m, 2H), 1.94-1.87 (m, 1H), 1.78-1.69 (m, 1H), 1.54 (dd, J=12.6, 6.3 Hz, 1H). MS m/z: [M+H]⁺ 349.07; HPLC>95%.

### Example 15: Synthesis of Compound 17

The synthetic route of (1R,5S,6r)-N-((S)-1-(8-bromoimidazo[1,5-a]pyridin-3-yl)ethyl)-3-azabicyclo[3.1.1]heptane-6-carboxamide Compound 17 is shown in Reaction Scheme 15.

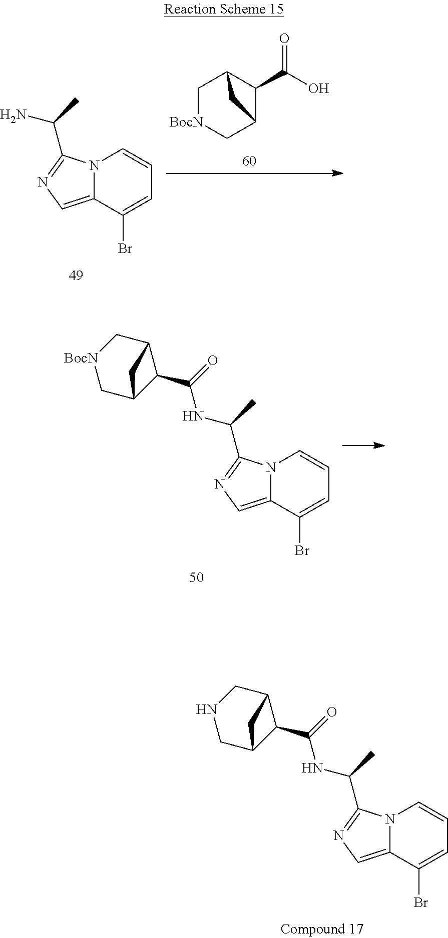 [SMILES: C[C@H](N)c1ncc2c(Br)cccn12 ; CC(C)(C)OC(=O)N1C[C@H]2C[C@@H](C1)[C@@H]2C(=O)O ; C[C@H](NC(=O)[C@@H]1[C@@H]2C[C@H]1CN(C(=O)OC(C)(C)C)C2)c1ncc2c(Br)cccn12 ; C[C@H](NC(=O)[C@H]1[C@@H]2CNC[C@H]1C2)c1ncc2c(Br)cccn12]

Step 1: Synthesis of tert-butyl (1R,5S,6r)-6-(((S)-1-(8-bromoimidazo[1,5-a]pyridin-3-yl)ethyl)carbamoyl)-3-azabicyclo[3.1.1]heptane-3-carboxylate Compound 17

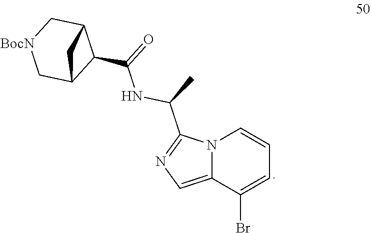 [SMILES: C[C@H](NC(=O)[C@@H]1[C@@H]2C[C@H]1CN(C(=O)OC(C)(C)C)C2)c1ncc2c(Br)cccn12]

(S)-1-(8-bromoimidazo[1,5-a]pyridin-3-yl)ethan-1-amine 50 (500 mg, 2.1 mmol) was added to a 100 mL round-bottom flask followed by the addition of 10 mL DCM as a solvent. To the mixture, (1R,5S,6r)-3-(tert-butoxycarbonyl)-3-azabicyclo[3.1.1]heptane-6-carboxylic acid 60 (554 mg, 2.3 mmol) and HATU (951 mg, 2.5 mmol) were added. Finally, TEA (5 mL) was added and stired at room temperature for 1 hour. LC-MS showed complete reaction of the starting material. The mixture was extracted using a DCM and NaHSO₄ solution. The organic layer was dried over anhydrous Na₂SO₄ and was concentrated. The product was purified by column chromatography to obtain tert-butyl (1R,5S,6r)-6-(((S)-1-(8-bromoimidazo[1,5-a]pyridin-3-yl)ethyl)carbamoyl)-3-azabicyclo[3.1.1]heptane-3-carboxylate 50 (778 mg, 80%). MS m/z: [M+H]⁺ 463.13.

Step 2: Synthesis of (1R,5S,6r)-N-((S)-1-(8-bromoimidazo[1,5-a]pyridin-3-yl)ethyl)-3-azabicyclo[3.1.1]heptane-6-carboxamide Compound 17

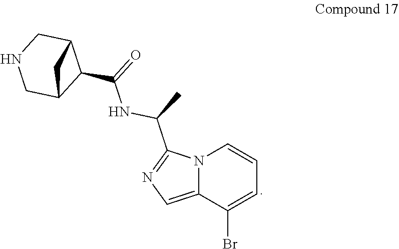 [SMILES: C[C@H](NC(=O)[C@H]1[C@@H]2CNC[C@H]1C2)c1ncc2c(Br)cccn12]

tert-butyl (1R,5S,6r)-6-(((S)-1-(8-bromoimidazo[1,5-a]pyridin-3-yl)ethyl)carbamoyl)-3-azabicyclo[3.1.1]heptane-3-carboxylate 50 (778 mg, 1.7 mmol) was added to a 50 mL round-bottom flask followed by the addition of EtOH/HCl (5 mL) and the mixture was stirred at room temperature for 30 minutes. Vacuum distillation was performed to obtain (1R,5S,6r)-N-((S)-1-(8-bromoimidazo[1,5-a]pyridin-3-yl)ethyl)-3-azabicyclo[3.1.1]heptane-6-carboxamide Compound 17 (556 mg, 90%). ¹H NMR (500 MHz, Chloroform-d) δ 9.26 (dd, J=8.4, 1.3 Hz, 1H), 7.70 (dd, J=7.6, 1.5 Hz, 1H), 7.54 (s, 1H), 7.21 (d, J=8.2 Hz, 1H), 7.11 (dd, J=8.3, 7.4 Hz, 1H), 5.05-4.97 (m, 1H), 3.06-2.96 (m, 4H), 2.56 (t, J=7.4 Hz, 1H), 2.31-2.21 (m, 2H), 1.94-1.87 (m, 1H), 1.78-1.69 (m, 1H), 1.63 (s, 1H), 1.58-1.49 (m, 1H). MS m/z: [M+H]⁺363.08; HPLC>95%.

### Example 16: Synthesis of Compound 18

The synthetic route of (1R,5S,6r)-N-((R)-1-(8-bromoimidazo[1,5-a]pyridin-3-yl)ethyl)-3-azabicyclo[3.1.1]heptane-6-carboxamide Compound 18 is shown in Reaction Scheme 16.

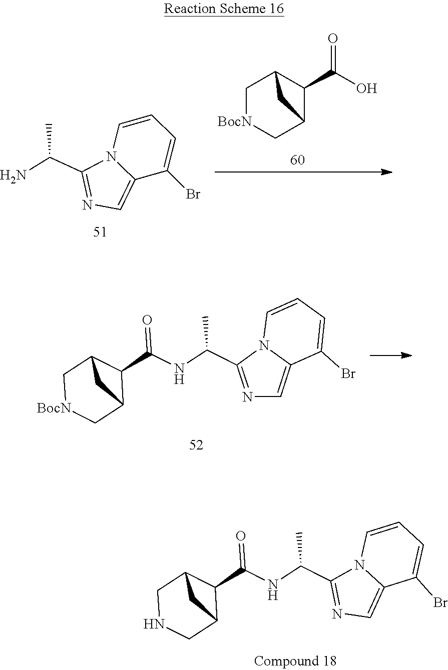 [SMILES: C[C@@H](N)c1ncc2c(Br)cccn12 ; CC(C)(C)OC(=O)N1C[C@H]2C[C@@H](C1)[C@@H]2C(=O)O ; C[C@@H](NC(=O)[C@@H]1[C@@H]2C[C@H]1CN(C(=O)OC(C)(C)C)C2)c1ncc2c(Br)cccn12 ; C[C@@H](NC(=O)[C@H]1[C@@H]2CNC[C@H]1C2)c1ncc2c(Br)cccn12]

Step 1: Synthesis of tert-butyl (1R,5S,6r)-6-(((R)-1-(8-bromoimidazo[1,5-a]pyridin-3-yl)ethyl)carbamoyl)-3-azabicyclo[3.1.1]heptane-3-carboxylate 52

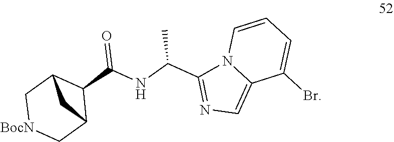 [SMILES: C[C@@H](NC(=O)[C@@H]1[C@@H]2C[C@H]1CN(C(=O)OC(C)(C)C)C2)c1ncc2c(Br)cccn12]

(R)-1-(8-bromoimidazo[1,5-a]pyridin-3-yl)ethan-1-amine 51 (500 mg, 2.1 mmol) was added to a 100 mL round-bottom flask followed by the addition of 10 mL DCM as a solvent. To the mixture, (1R,5S,6r)-3-(tert-butoxycarbonyl)-3-azabicyclo[3.1.1]heptane-6-carboxylic acid (554 mg, 2.3 mmol) 60 and HATU (951 mg, 2.5 mmol) were added. Finally TEA (5 mL) was added and the mixture stirred at room temperature for 1 hour. LC-MS showed complete reaction of the starting material. The mixture was extracted with DCM and NaHSO₄ solution. The organic layer was dried over anhydrous Na₂SO₄ and was concentrated. The product was purified by column chromatography to obtain tert-butyl (1R,5S,6r)-6-(((R)-1-(8-bromoimidazo[1,5-a]pyridin-3-yl)ethyl)carbamoyl)-3-azabicyclo[3.1.1]heptane-3-carboxylate 52 (778 mg, 80%). MS m/z: [M+H]⁺ 463.13.

Step 2: Synthesis of (1R,5S,6r)-N-((R)-1-(8-bromoimidazo[1,5-a]pyridin-3-yl)ethyl)-3-azabicyclo[3.1.1]heptane-6-carboxamide Compound 18

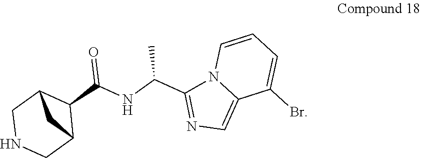 [SMILES: C[C@@H](NC(=O)[C@H]1[C@@H]2CNC[C@H]1C2)c1ncc2c(Br)cccn12]

tert-butyl (1R,5S,6r)-6-(((R)-1-(8-bromoimidazo[1,5-a]pyridin-3-yl)ethyl)carbamoyl)-3-azabicyclo[3.1.1]heptane-3-carboxylate 52 (778 mg, 1.7 mmol) was added to a 50 mL round-bottom flask followed by the addition of EtOH/HCl (5 mL). The mixture was stirred at room temperature for 30 minutes, then vacuum distillation was performed to obtain the product (1R,5S,6r)-N-((R)-1-(8-bromoimidazo[1,5-a]pyridin-3-yl)ethyl)-3-azabicyclo[3.1.1]heptane-6-carboxamide Compound 18 (556 mg, 90%). ¹H NMR (500 MHZ, Chloroform-d) δ 9.26 (dd, J=8.4, 1.3 Hz, 1H), 7.70 (dd, J=7.6, 1.5 Hz, 1H), 7.54 (s, 1H), 7.21 (d, J=8.2 Hz, 1H), 7.11 (dd, J=8.3, 7.4 Hz, 1H), 5.05 -4.97 (m, 1H), 3.06-2.96 (m, 4H), 2.56 (t, J=7.4 Hz, 1H), 2.31-2.21 (m, 2H), 1.94 -1.87 (m, 1H), 1.78-1.69 (m, 1H), 1.63 (s, 1H), 1.58-1.49 (m, 1H). MS m/z: [M+H]⁺ 363.08; HPLC>95%.

### Example 17: Synthesis of Compound 19

The synthetic route of (1R,5S,6r)-N-(2-(8-chloroimidazo[1,5-a]pyridin-3-yl)propan-2-yl)-3-formyl-3-azabicyclo[3.1.1]heptane-6-carboxamide Compound 19 is shown in Reaction Scheme 17.

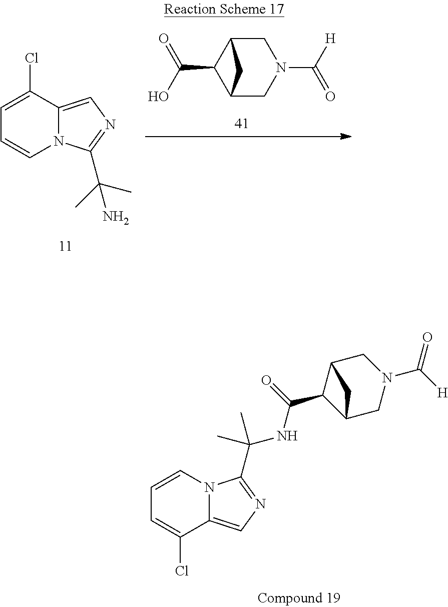 [SMILES: CC(C)(N)c1ncc2c(Cl)cccn12 ; O=CN1C[C@H]2C[C@@H](C1)[C@@H]2C(=O)O ; CC(C)(NC(=O)[C@@H]1[C@@H]2C[C@H]1CN(C=O)C2)c1ncc2c(Cl)cccn12]

Step 1: Synthesis of (1R,5S,6r)-N-(2-(8-chloroimidazo[1,5-a]pyridin-3-yl)propan-2-yl)-3-formyl-3-azabicyclo[3.1.1]heptane-6-carboxamide Compound 19

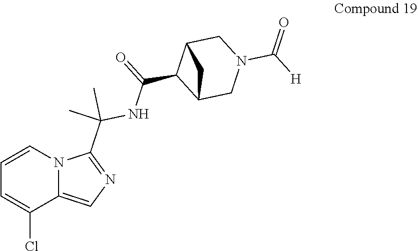 [SMILES: CC(C)(NC(=O)[C@@H]1[C@@H]2C[C@H]1CN(C=O)C2)c1ncc2c(Cl)cccn12]

2-(8-chloroimidazo[1,5-a]pyridin-3-yl)propan-2-amine 11 (500 mg, 2.4 mmol) was added to a 100 mL round-bottom flask followed by the addition of 10 mL of DCM as a solvent. To the mixture, (1R,5S,6r)-3-formyl-3-azabicyclo[3.1.1]heptane-6-carboxylic acid 41 (447 mg, 2.6 mmol) and HATU (1.1 g, 2.9 mmol) were added. Finally, TEA (5 mL) was added and stirred at room temperature for 1 hour. LC-MS showed complete reaction of the starting material. The mixture was extracted with a DCM and NaHSO₄ solution. The organic layer was dried over anhydrous Na₂SO₄ and the organic layer was concentrated. The product was purified by column chromatography to obtain (1R,5S,6r)-N-(2-(8-chloroimidazo[1,5-a]pyridin-3-yl)propan-2-yl)-3-formyl-3-azabicyclo[3.1.1]heptane-6-carboxamide Compound 19 (693 mg, 80%). ¹H NMR (500 MHz, Chloroform-d) δ 9.18 (dd, J=7.3, 1.3 Hz, 1H), 8.26 (s, 1H), 7.78 (s, 1H), 7.59 (dd, J=7.4, 1.5 Hz, 1H), 7.15-7.08 (m, 1H), 6.98 (s, 1H), 3.72 (dd, J=12.4, 2.0 Hz, 2H), 3.57 (dd, J=12.3, 2.0 Hz, 2H), 2.66-2.60 (m, 1H), 2.58-2.48 (m, 2H), 1.85-1.76 (m, 1H), 1.65-1.56 (m, 1H). MS m/z: [M+H]⁺ 361.14; HPLC>95%.

### Example 18: Synthesis of Compound 21

The synthetic route of (1R,5S,6r)-3-carbamimidoyl-N-(2-(8-chloroimidazo[1,5-a]pyridin-3-yl)propan-2-yl)-3-azabicyclo[3.1.1]heptane-6-carboxamide Compound 21 is shown in Reaction Scheme 18.

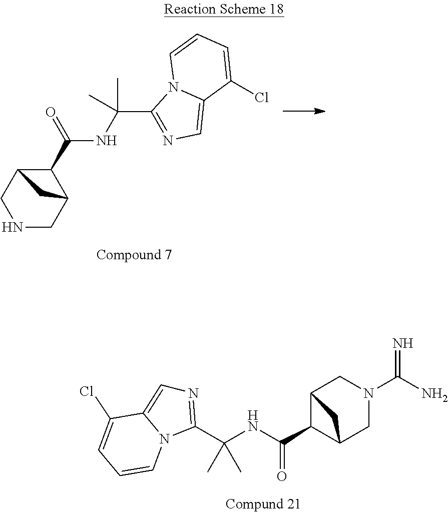 [SMILES: CC(C)(NC(=O)[C@H]1[C@@H]2CNC[C@H]1C2)c1ncc2c(Cl)cccn12 ; CC(C)(NC(=O)[C@@H]1[C@@H]2C[C@H]1CN(C(=N)N)C2)c1ncc2c(Cl)cccn12]

Step 1: Synthesis of (1R,5S,6r)-3-carbamimidoyl-N-(2-(8-chloroimidazo[1,5-a]pyridin-3-yl)propan-2-yl)-3-azabicyclo[3.1.1]heptane-6-carboxamide Compound 21

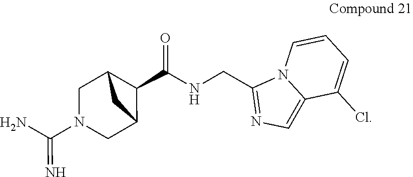 [SMILES: N=C(N)N1C[C@H]2C[C@@H](C1)[C@@H]2C(=O)NCc1ncc2c(Cl)cccn12]

(1R,5S,6r)-N-(2-(8-chloroimidazo[1,5-a]pyridin-3-yl)prop-2-yl)-3-azabicyclo[3.1.1]heptane-6-carboxamide Compound 7 (500 mg, 1.5 mmol) was added to a 100 mL round-bottom flask followed by the addition of 1H-pyrazole-1-methanol hydrochloride (263 mg, 1.8 mmol) and DIPEA (4 mL). The mixture was stirred at room temperature for 1 hour. The completion of the reaction was confirmed by by LC-MS. The mixture was extracted with DCM and NaHSO₄ solution. The organic layer was dried over anhydrous Na₂SO₄, concentrated, and purified by column chromatography to obtain (1R,5S,6r)-3-carbamimidoyl-N-(2-(8-chloroimidazo[1,5-a]pyridin-3-yl)propan-2-yl)-3-azabicyclo[3.1.1]heptane-6-carboxamide Compound 21 (337 mg, 60%). ¹H NMR (500 MHZ, Chloroform-d) δ 9.18 (dd, J=7.3, 1.3 Hz, 1H), 8.26 (s, 1H), 7.59 (dd, J=7.4, 1.5 Hz, 1H), 7.15-7.06 (m, 2H), 6.98 (s, 1H), 5.51 (s, 2H), 3.72 (dd, J=12.2, 1.6 Hz, 2H), 3.57 (dd, J=12.4, 1.6 Hz, 2H), 2.64 (t, J=6.7 Hz, 1H), 2.40-2.31 (m, 2H), 1.88-1.79 (m, 1H), 1.62-1.53 (m, 1H). MS m/z: [M+H]⁺375.17; HPLC>95%.

### Example 19: Synthesis of Compound 22

The synthetic route of (1R,5S,6r)-3-carbamimidoyl-N-(2-(1-cyclopropylimidazo[1,5-a]pyridin-3-yl)propan-2-yl)-3-azabicyclo[3.1.1]heptane-6-carboxamide Compound 22 is shown in Reaction Scheme 19.

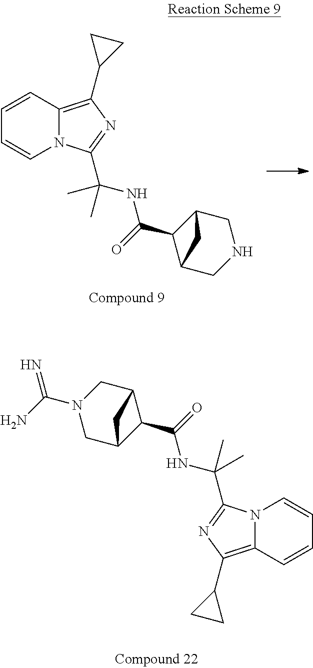 [SMILES: CC(C)(NC(=O)[C@H]1[C@@H]2CNC[C@H]1C2)c1nc(C2CC2)c2ccccn12 ; CC(C)(NC(=O)[C@@H]1[C@@H]2C[C@H]1CN(C(=N)N)C2)c1nc(C2CC2)c2ccccn12]

Step 1: Synthesis of (1R,5S,6r)-3-carbamimidoyl-N-(2-(1-cyclopropylimidazo[1,5-a]pyridin-3-yl)propan-2-yl)-3-azabicyclo[3.1.1]heptane-6-carboxamide Compound 22

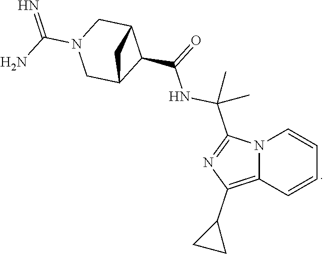 [SMILES: CC(C)(NC(=O)[C@@H]1[C@@H]2C[C@H]1CN(C(=N)N)C2)c1nc(C2CC2)c2ccccn12]

(1R,5S,6r)-N-(2-(1-cyclopropylimidazo[1,5-a]pyridin-3-yl)propan-2-yl)-3-azabicyclo[3.1.1]heptane-6-carboxamide Compound 9 (500 mg, 1.5 mmol) was added to a 100 mL round-bottom flask followed by the addition of 1H-pyrazole-1-methanol hydrochloride (264 mg, 1.8 mmol) and DIPEA (4 mL). The mixture was stirred at room temperature for 1 hour. The completion of the reaction was confirmed by by LC-MS. The mixture was extracted with DCM and NaHSO₄ solution. The organic layer was dried over anhydrous Na₂SO₄, concentrated, and purified by column chromatography to obtain (1R,5S,6r)-3-carbamimidoyl-N-(2-(1-cyclopropylimidazo[1,5-a]pyridin-3-yl)propan-2-yl)-3-azabicyclo[3.1.1]heptane-6-carboxamide Compound 22 (342 mg, 60%). ¹H NMR (500 MHZ, Chloroform-d) δ 8.82 (dd, J=7.2, 1.3 Hz, 1H), 7.63 (dd, J=9.6, 1.5 Hz, 1H), 7.28 (s, 1H), 7.22-7.15 (m, 1H), 7.06-6.96 (m, 2H), 5.51 (s, 2H), 3.72 (dd, J=12.2, 1.6 Hz, 2H), 3.57 (dd, J=12.4, 1.6 Hz, 2H), 2.83-2.76 (m, 1H), 2.64 (t, J=6.7 Hz, 1H), 2.40-2.31 (m, 2H), 1.88-1.79 (m, 1H), 1.62-1.53 (m, 1H), 1.08-0.94 (m, 5H). MS m/z: [M+H]⁺ 381.24; HPLC>95%.

### Example 20: Synthesis of Compound 23

The synthetic route of (1R,5S,6r)-N-(2-(8-(methylselanyl)imidazo[1,5-a]pyridin-3-yl)propan-2-yl)-3-azabicyclo[3.1.1]heptane-6-carboxamide Compound 23 is shown in Reaction Scheme 20

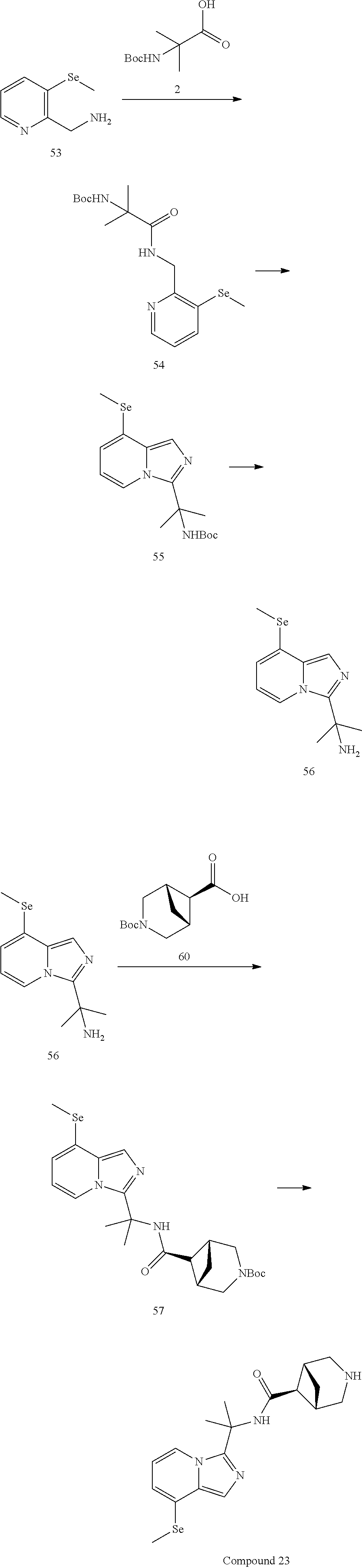

Step 1: Synthesis of tert-butyl (2-methyl-1-(((3-(methylselanyl)pyridin-2-yl)methyl)amino)-1-oxopropan-2-yl)carbamate 54

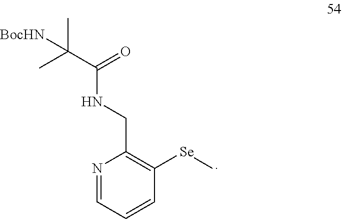 [SMILES: C[Se]c1cccnc1CNC(=O)C(C)(C)NC(=O)OC(C)(C)C]

(3-(methylselanyl)pyridin-2-yl)methanamine (2 g, 9.9 mmol) was added to a 100 mL round-bottom flask, followed by the addition of 20 mL of DCM as a solvent. To the mixture, 2-((tert-butoxycarbonyl)amino)-2-methylpropanoic acid (2.42 g, 11.9 mmol) and HATU (4.52 g, 11.9 mmol) were added. Finally, TEA (10 mL) was added to the mixture and the mixture was stirred at room temperature for 1 hour. LC-MS indicated complete reaction of the starting materials. The mixture was extracted with DCM and NaHSO₄ solution, the organic layer was dried with anhydrous Na₂SO₄, and concentrated to obtain tert-butyl (2-methyl-1-(((3-(methylselanyl)pyridin-2-yl)methyl)amino)-1-oxopropan-2-yl)carbamate (3.8 g, 100%). MS m/z: [M+H]+ 388.11 54.

Step 2: Synthesis of tert-butyl (2-(8-(methylselanyl)imidazo[1,5-a]pyridin-3-yl)propan-2-yl)carbamate 55

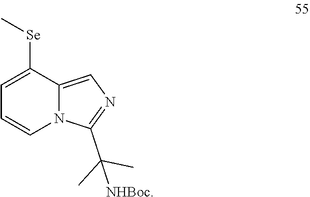 [SMILES: C[Se]c1cccn2c(C(C)(C)NC(=O)OC(C)(C)C)ncc12]

tert-butyl (2-methyl-1-(((3-(methylselanyl)pyridin-2-yl)methyl)amino)-1-oxopropan-2-yl)carbamate 54 (3.8 g, 9.9 mmol) was weighed, added to a 250 ml round-bottom flask, and dissolved in 60 mL of DCM. An appropriate amount of molecular sieves were added, the reaction mixture was stirred for 30 minutes, and Burgess reagent (3.4 g, 14.8 mmol) was added to the mixture. The reaction mixture was stirred for 2 hours. LC-MS indicated complete reaction of the starting materials. The mixture was filtered, and the filtrate was directly subjected to column chromatography for purification to obtain tert-butyl (2-(8-(methylselanyl)imidazo[1,5-a]pyridin-3-yl)propan-2-yl)carbamate (1.1 g, 30%) 55. MS m/z: [M+H]+ 370.10.

Step 3: Synthesis of 2-(8-(methylselanyl)imidazo[1,5-a]pyridin-3-yl)propan-2-amine 56

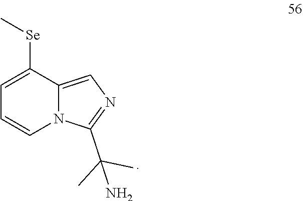

tert-butyl (2-(8-(methylselanyl)imidazo[1,5-a]pyridin-3-yl)propan-2-yl)carbamate 55 (1.1 g, 3.0 mmol) and EtOH/HCl (5 mL) were added to a 50 mL round-bottom flask. The reaction mixture was stirred at room temperature for 30 minutes, then vacuum distillation was performed to obtain 2-(8-(methylselanyl)imidazo[1,5-a]pyridin-3-yl)propan-2-amine 56 (724 mg,90%). MS m/z: [M+H]+270.05.

Step 4: Synthesis of tert-butyl (1R,5S,6r)-6-((2-(8-(methylselanyl)imidazo[1,5-a]pyridin-3-yl)propan-2-yl)carbamoyl)-3-azabicyclo[3.1.1]heptane-3-carboxylate 57

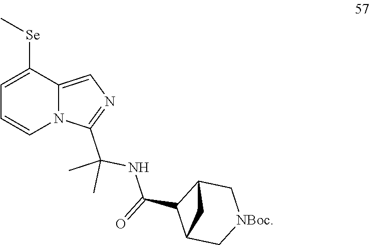

2-(8-(methylselanyl)imidazo[1,5-a]pyridin-3-yl)propan-2-amine (724 mg, 2.7 mmol) and 20 mL of DCM as a solvent were added to a 100 mL round-bottom flask. (1R,5S,6r)-3-(tert-butoxycarbonyl)-3-azabicyclo[3.1.1]heptane-6-carboxylic acid 60 (723 mg, 3.0 mmol) and HATU (1.2 g, 3.2 mmol) were added to the reaction mixture. Finally, TEA (5 mL) was added and stirred at room temperature for 1 hour. LC-MS indicated the complete reaction of the starting materials. The mixture was extracted with a DCM and NaHSO₄ solution, the organic layer was dried with anhydrous Na2SO4, concentrated, and purified by column chromatography to obtain tert-butyl (1R,5S,6r)-6-((2-(8-(methylselanyl)imidazo[1,5-a]pyridin-3-yl)propan-2-yl)carbamoyl)-3-azabicyclo[3.1.1]heptane-3-carboxylate 57 (1.1 g, 80%). MS m/z: [M+H]+ 493.17.

Step 5: Synthesis of (1R,5S,6r)-N-(2-(8-(methylselanyl)imidazo[1,5-a]pyridin-3-yl)propan-2-yl)-3-azabicyclo[3.1.1]heptane-6-carboxamide Compound 23

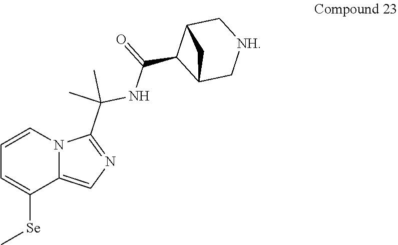 [SMILES: C[Se]c1cccn2c(C(C)(C)NC(=O)[C@H]3[C@@H]4CNC[C@H]3C4)ncc12]

tert-butyl (1R,5S,6r)-6-((2-(8-(methylselanyl)imidazo[1,5-a]pyridin-3-yl)propan-2-yl)carbamoyl)-3-azabicyclo[3.1.1]heptane-3-carboxylate (1.1 g, 2.2 mmol) and EtOH/HCl (10 mL) were added to a 50 mL round-bottom flask. The reaction mixture was stirred at room temperature for 30 minutes, then vacuum distillation was performed to obtain (1R,5S,6r)-N-(2-(8-(methylselanyl)imidazo[1,5-a]pyridin-3-yl)propan-2-yl)-3-azabicyclo[3.1.1]heptane-6-carboxamide Compound 23 (775 mg,90%). ¹H NMR (500 MHz, Chloroform-d) δ 9.05 (dd, J=7.0, 1.5 Hz, 1H), 7.98 (s, 1H), 7.55 (s, 1H), 7.49 (dd, J=7.9, 1.5 Hz, 1H), 7.21 (dd, J=7.9, 7.1 Hz, 1H), 3.01 (t, J=5.6 Hz, 1H), 2.95 (m, J=12.2, 5.0, 4.1 Hz, 2H), 2.86 (m, J=12.2, 5.0, 4.2 Hz, 2H), 2.66 (q, J=5.0 Hz, 1H), 2.66-2.55 (m, 2H), 2.53 (s, 2H), 2.09-1.98 (m, 2H), 1.92 (s, 4H). MS m/z: [M+H]⁺ 393.12.

### Biological Study and Testing

### Example 21: Assessing Affinity in Radioactive Ligand Binding

The assessment of affinity in radioactive ligand binding was carried out at Eurofins, following the methodology described in the reference (ROHRER, L., RAULF, F., BRUNS, C., BUETTNER, R., HOFSTAEDTER, F. and SCHÜLE, R. (1993), Cloning and characterization of the fourth human somatostatin receptor, Proc. Natl. Acad. Sci. USA, 90: 4196). The detailed procedures are outlined as follows.

### SSTR-1 Binding Assay

In a buffer containing 25 mM Hepes/Tris (pH 7.4), 5 mM MgCl₂, 1 mM CaCl₂, and 0.5% BSA, cell membrane homogenates (5 μg protein) were incubated at 37° C. for 180 minutes with 0.1 nM [125I]Tyr11-somatostatin-14 in the absence or presence of test compounds. Non-specific binding was determined in the presence of 1 μM somatostatin-28. Following incubation, samples were swiftly vacuum-filtered through glass fiber filters pre-soaked with 0.3% PEI (GF/B, Packard), and washed multiple times with ice-cold 50 mM Tris-HCl using a 96-sample cell harvester (Unifilter, Packard). The filters were dried, then subjected to radioactive counting using a liquid scintillation counter (Topcount, Packard) with scintillation fluid (Microscint 0, Packard). Results are expressed as the percentage inhibition of specific binding of the control radioactive ligand. The standard reference compound is native somatostatin-28, with multiple concentrations tested in each experiment.

### SSTR-4 Binding Assay

In a buffer containing 50 mM Tris-HCl (pH 7.4), 5 mM MgCl₂, and 0.2% BSA, cell membrane homogenates (8 μg protein) were incubated at 22° C. for 120 minutes with 0.1 nM [125I]Tyr11-somatostatin-14 in the absence or presence of test compounds. Non-specific binding was determined in the presence of 1 μM somatostatin-14. After incubation, samples were rapidly filtered under vacuum through glass fiber filters pre-soaked with 0.3% PEI (GF/B, Packard) and washed several times with ice-cold buffer containing 50 mM Tris-HCl and 0.5% BSA using a 96-sample cell harvester (Unifilter, Packard). The filters were dried, then subjected to radioactive counting using a liquid scintillation counter (Topcount, Packard) with a scintillation mixture (Microscint 0, Packard). Results are expressed as the percentage inhibition of specific binding of the control radioactive ligand. The standard reference compound is somatostatin-14, with multiple concentrations tested in each experiment.

TABLE 1
Inhibition Rates on SSTR1 and SSTR4 Receptors
at 0.1 μM and 30 μM

Compound 15

| Receptor | 0.1 μM | 30 μM |
|---|---|---|
| SSTR1 | −0.28% | 39.97% |
| SSTR4 | 47.55% | 94.02% |

### Example 22: FLIPR Assay for Assessing Agonists of SSTR1-5

The study was carried out at HD Biosciences (Shanghai, China) according to the following steps.

Step 1: Test compounds were diluted to a 400×stock solution in DMSO in a 384-well plate.

Step 2: 1 μL of the compound solution was transferred from Step 1 to 79 μL of assay buffer, creating a 5×working solution in the 384-well plate using the Bravo method.

Step 3: HEK293/Gα15/SSTR1-5 cells were cultured in DMEM medium (10% FBS).

Step 4: When cells reached 80% confluence, the cells were separated using 0.25% trypsin-EDTA.

Step 5: The cell density was measured and the cells diluted to 1×10\hat{ }6/mL using DMEM (10% FBS).

Step 6: 30 μL of cells were dispensed into each well of a Matrigel-coated 384-well plate (Corning 3764#) using a multi-channel pipette (30,000 cells per well) and cultured for 20-24 hours at 37° C. and 5% CO2.

Step 7: 10 μL of dye was added to each well in the cell plate prepared in Step 6.

Step 8: The dye and cells were incubated in the dark at 37° C. and 5% CO₂ for 1 hour.

Step 9: After 1 hour, the FLIPR program was loaded, the pipette tips placed cell plate and compound plate (from Step 2) into the machine, and the run button was pressed. The program added 10 μL to 40 μL to the cell plate. FLIPR was used to read the plate at room temperature according to the specified settings and the data was saved.

TABLE 2
SSTR1-5 Agonist Activity in FLIPR Assay

| | EC₅₀ (nM) | | | | |
|---|---|---|---|---|---|
| Compound | SSTR1 | SSTR2 | SSTR3 | SSTR4 | SSTR5 |
| 3 | — | — | — | >1000 | — |
| 7 | >1000 | >1000 | >1000 | 4.16 | >1000 |
| 8 | >1000 | >1000 | >1000 | 3.33 | >1000 |
| 15 | >1000 | >1000 | >1000 | 6.64 | >1000 |
| Somatostatin-14 | 149.39 | 164.00 | 77.7 | 519.22 | 108.3 |

### Example 23: Acetic Acid Induced Writhing Test

The test compound was administered to KM mice (provided by Hunan Silaikejingda Experimental Animal Co.,Ltd.) via subcutaneous injection at a predetermined dosage of 5 mL/kg. Fifteen minutes after administration, a 0.6% acetic acid solution (5 mL/kg) was injected into the abdominal cavity. The mice were then placed in an observation box, and the number of writhing episodes was recorded within a 30-minute period. The assessment of pain intensity was based on the observation of the mice's writhing behavior.

TABLE 3
Analgesic Effects of Compounds in Acetic Acid Induced Writhing Test

| | | | | | Percentage | Significant |
|---|---|---|---|---|---|---|
| | | Administration | No. of | No. of | of | compared to the |
| Compound | Dosage | Method | animals | writhing's | Inhibition | model group |
| 7 | 30 | s.c. | 12 | 10 | 69.16 | Yes |
| | mg/kg | | | | | |
| 8 | 30 | s.c. | 8 | 3 | 86.26 | Yes |
| | mg/kg | | | | | |
| 9 | 30 | s.c. | 7 | 8 | 62.34 | Yes |
| | mg/kg | | | | | |
| 12 | 30 | s.c. | 7 | 10 | 54.16 | Yes |
| | mg/kg | | | | | |
| 20 | 30 | s.c. | 12 | 3 | 88.80 | Yes |
| | mg/kg | | | | | |
| 21 | 30 | s.c. | 12 | 9 | 71 | Yes |
| | mg/kg | | | | | |
| 22 | 30 | s.c. | 12 | 7 ± 2 | 82.45 ± 5.13 | Yes |
| | mg/kg | | | | | |

### Example 24: SD Rat SNL model

The Spinal Nerve Ligation (SNL) model involves the tight ligation of the L5 and L6 spinal nerves using sutures. Following the surgical procedure, animals exhibit apparent neuropathic pain for 7-14 days, characterized by sensory hypersensitivity in the hind paw on the side of the ligation. Rats were subjected to surgery to induce tactile allodynia, and 7 days after surgical recovery, baseline mechanical threshold testing was performed using Von Frey filaments (Shanghai Yuyan Scientific Instrument Co., Ltd., North Coast). Rats that demonstrated successful modeling were then selected for efficacy testing. After initial screening, rats were sequentially numbered and randomly assigned to groups. Experimental groups were established based on the trial conditions. SD rats (provided by Hunan Silaikejingda Experimental Animal Co.,Ltd.) in each group were administered the respective drugs, and their mechanical pain thresholds on the modeled side of the hind paw were measured at 1, 2, and 4 hours post-administration, with the results recorded.

TABLE 4
Analgesic Effects of Compounds in the
Spinal Nerve Ligation (SNL) Model

| | | Administration | |
|---|---|---|---|
| Compound | Dosage | Method | Experimental results |
| 8 | 30 mg/kg | s.c. | Demonstrated a pain-relieving |
| | | | effect that was stronger than |
| | | | naproxen |
| Naproxen | 30 mg/kg | p.o. | Indicated a certain degree of |
| | | | analgesic efficacy |

### Example 25: SD Rat Carrageenan Model

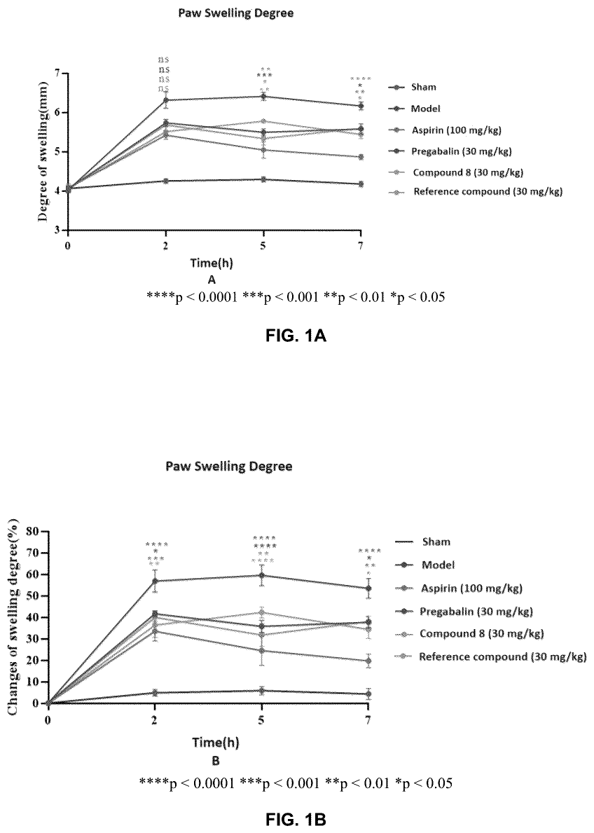

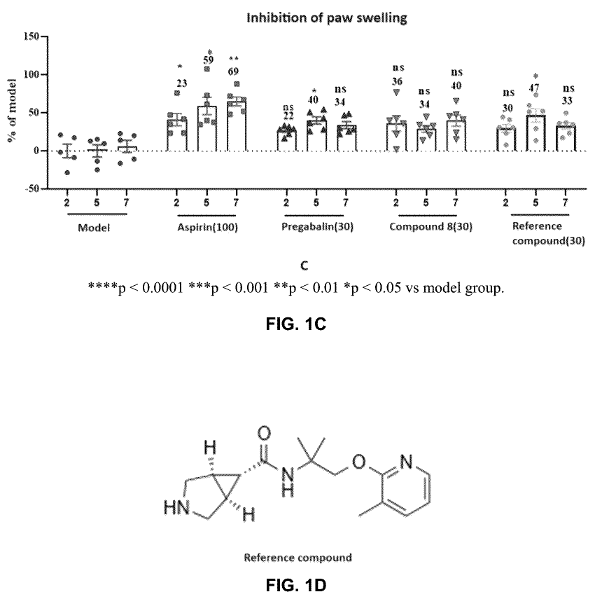

In SD rats (provided by Hunan Silaikejingda Experimental Animal Co., Ltd.), markings were made at the same location on the footpad. Using an electronic caliper for accuracy, the thickness of the rat's footpad was measured. Rats were then restrained, and 100 μl of 1% carrageenan was subcutaneously injected into the right hind footpad to induce inflammation, thereby establishing an acute inflammation model using carrageenan. Each compound was administered in advance according to the respective dosing time points. The swelling of the rat's foot was measured at various time points: before modeling (0 h) and after modeling (2, 4, 7 h). A comparison was made between footpad swelling before and after modeling. The results under the conditions of this experiment are presented in Table 5 and Table 6. Notably, a single subcutaneous injection of 30 mg/kg of compound 8 demonstrated anti-inflammatory tendencies. The results of the carrageenan-induced acute inflammation model experiment are depicted in FIG. 1.

TABLE 5
Foot Swelling Degrees in the Modeling
Groups (mm, Mean ± SEM)

| Group | 0 h | 2 h | 4 h | 7 h |
|---|---|---|---|---|
| Sham | 4.06 ± 0.06 | 4.25 ± 0.06 | 4.30 ± 0.07 | 4.18 ± 0.08 |
| Model | 4.03 ± 0.08 | 6.32 ± 0.21 | 6.41 ± 0.10 | 6.17 ± 0.10 |
| Aspirin | 4.08 ± 0.10 | 5.42 ± 0.11 | 5.05 ± 0.21 | 4.87 ± 0.07 |
| Compound 8 | 4.02 ± 0.09 | 5.51 ± 0.13 | 5.78 ± 0.05 | 5.44 ± 0.11 |
| Reference | 4.06 ± 0.08 | 5.68 ± 0.15 | 5.34 ± 0.16 | 5.60 ± 0.11 |
| compound | | | | |
| Pregabalin | 4.06 ± 0.07 | 5.74 ± 0.09 | 5.49 ± 0.09 | 5.58 ± 0.13 |

TABLE 6
Changes in Foot Swelling Rate in the
Modeling Groups (%, Mean ± SEM)

| Group | 0 h | 2 h | 4 h | 7 h |
|---|---|---|---|---|
| Sham | 0 | 4.81 ± 1.58 | 5.84 ± 1.92 | 4.31 ± 2.58 |
| Model | 0 | 56.86 ± 5.14 | 59.50 ± 4.79 | 53.45 ± 4.63 |
| Aspirin | 0 | 33.44 ± 4.56 | 24.42 ± 6.86 | 19.72 ± 3.24 |
| Compound 8 | 0 | 36.28 ± 5.75 | 42.25 ± 2.63 | 34.30 ± 4.17 |
| Reference compound | 0 | 39.92 ± 3.05 | 31.71 ± 5.25 | 38.01 ± 2.67 |
| Pregabalin | 0 | 41.54 ± 1.45 | 35.68 ± 2.85 | 37.64 ± 2.63 |

### Example 26: SD Rat CCI Model

The chronic constriction injury of the sciatic nerve (CCI) in SD rats (provided by Hunan Silaikejingda Experimental Animal Co., Ltd.) is a traditional model used to study neuropathic pain. This model involves the use of a non-absorbable 5-0 suture to loosely ligate the sciatic nerve, causing slight compression of its outer sheath. Mechanical compression by the suture induces axonal damage, leading to abnormal discharge. Additionally, the local stimulation from the ligature induces the release of inflammatory mediators.

Furthermore, CCI selectively damages the larger myelinated fibers of the sciatic nerve while preserving most of the unmyelinated C fibers responsible for transmitting pain. Initially, rats undergo surgery to induce tactile allodynia, and 7 days after surgical recovery, baseline mechanical threshold testing is conducted using Von Frey filaments (Shanghai Yuyan Scientific Instrument Co., Ltd., North Coast). Rats demonstrating successful modeling are selected for efficacy testing.

After initial screening, rats are sequentially numbered and randomly assigned to groups based on the numbering, using a random grouping method. Experimental groups are established based on trial conditions. Rats in each group are administered the respective drugs, and the mechanical pain threshold on the modeled side of the hind paw is measured at 1, 2, and 4 hours post-administration, with the results recorded.

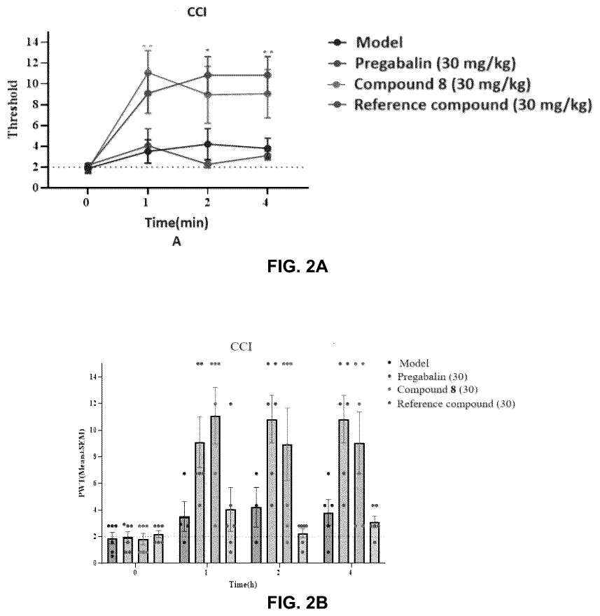

Under the conditions of this experiment, a single subcutaneous injection of compound 8 significantly affected the SD rat CCI model, as indicated in FIG. 2. The control compound did not exhibit a notable therapeutic effect on neuropathic pain, underlining the significant efficacy of compound 8.

### Example 27: In Vivo PK Study Results

TABLE 7
In vivo PK Experiment Results for Compound 8 and Compound 22

| | | | |
|---|---|---|---|
| compound | — | Compound 8 | Compound 22 |

| | | | | | |
|---|---|---|---|---|---|
| Dose | mg/kg | 1 | 3 | 1 | 3 |
| Rout | — | i.v. | p.o. | i.v. | p.o. |
| CL | L/hr/kg | 2.71 | — | — | — |
| Vₛₛ | L/kg | 2.38 | — | — | — |
| AUCₗₐₛₜ | hr*ng/mL | 361 | 87.3 | — | 1.3 |
| AUC_{INF} | hr*ng/mL | 370 | 38.4 | — | — |
| T_{1/2} | hr | 0.814 | 2.43 | — | — |
| MRT_{INF} | hr | 0.879 | — | — | 0.19 |
| Tₘₐₓ | hr | — | 1.42 | — | 0.19 |
| Cₘₐₓ | ng/mL | — | 36.3 | — | 5.56 |
| F | % | — | 8.06 | — | — |

### Example 28: In Vitro ADME Study Results

### Chemical Stability

1 mM test compound spiking solution (spiking solution A) was prepared by adding 10 μL of 10 mM a test compound stock solution to 90 μL of DMSO. 396 μL of buffer were added into tubes designated for different time points. The samples were pre-warmed at 37° C. for 10 minutes. 4 μL of spiking solution A was added into the wells containing 396 μL of buffer that were designated as 0 min, 15 min, 45 min, or 60 min. The timer was started after the addition of the spiking solution. At each time point, 1200 μL of acetonitrile (ACN) containing an internal standard was added to the tubes. The samples were centrifuged at 10,000 rpm for 5 minutes and 100 μL of the supernatant was removed and analyzed by for LC-MS/MS analysis. The results are in Table 8.

### Metabolic Stability

Buffers A-C were prepared for use in the metabolic study. The buffers were as follows: Buffer A: 1.0 L of 0.1 M monobasic Potassium Phosphate buffer containing 1.0 mM EDTA; Buffer B: 1.0 L of 0.1 M Dibasic Potassium Phosphate buffer containing 1.0 mM EDTA; and Buffer C: 0.1 M Potassium Phosphate buffer, 1.0 mM EDTA, pH 7.4 by titrating 700 mL of buffer B with buffer A while monitoring with the pH meter.

Ketanserin was used as the reference compound. Reference compound and test compound spiking solutions (500 μM spiking solution) were prepared by adding 10 μL of 10 mM DMSO stock solution into 190 μL ACN. 1.5 μL of 500 μM spiking solution (0.75 mg/mL) and 18.75 μL of 20 mg/mL liver microsomes were added into 479.75 μL of Buffer C on ice.

A 6 mM NADPH stock solution was prepared by dissolving an appropriate amount of NADPH into buffer C. 30 μL of 1.5 μM spiking solution containing 0.75 mg/mL microsomes solution was dispensed to the assay plates designated for different time points (0-, 5-, 15-, 30-, 45-min) on ice. For 0-min sample, 135 μL of ACN containing IS was added to the wells of 0-min plate and followed by 15 μL of NADPH stock solution (6 mM). All the other plates were pre-incubated at 37° C. for 5 minutes. 15 μL of 6 mM NADPH stock solution was added to the plates to start the reaction and time. At 5-min, 15-min, 30-min, and 45-min, 135 μL of ACN containing internal standard were added to the wells of corresponding plates, respectively, to stop the reaction. After the reaction was quenched, plates were stirred on a vibrator (IKA, MTS 2/4) for 10 min (600 rpm/min) and centrifuged at 5594 g for 15 min (Thermo Multifuge×3R). 50 μL of the supernatant was removed from each well and placed into a 96-well sample plate containing 50 μL of ultra pure water (Millipore, ZMQS50F01) for LC/MS analysis. The results are in Table 8.

### Protein Binding

### Spiking Solutions of Test and Reference Compounds

The following solutions were prepared: Solution A (0.5 mM) 10 μL of 10 mM stock solution was added into 190 μL of DMSO and Solution B (0.02 mM): 8 μL of Solution A was added into 192 μL of 0.05 M sodium phosphate buffer. The final DMSO concentration in Solution B is 4%.

Preparation of Test and Reference Compounds in plasma

A 96-well plate was preloaded with 380 μL aliquots of plasma in the wells designated for plasma and buffer, respectively. 20 μL of Solution B (0.02 mM of test and reference compounds) was spiked into the pre-loaded plasma in the 96-well plate. The final test concentration was 1 μM containing 0.2% DMSO.

### Dialysis Sample Loading

### Preparation of Plasma Against Buffer System (Duplicate)

Aliquots of 100 μL of blank dialysis buffer were applied to the receiver side of dialysis chambers. Next aliquots of 100 μL of the plasma spiked with test and reference compounds were applied to the donor side of the dialysis chambers. Blank buffer was added to the receiver first and the buffer and plasma chambers were clearly marked to avoid cross contamination.

### Preparation of Plasma Against Buffer System (Duplicate):

Aliquots of 100 μL of blank dialysis buffer were applied to the receiver side of dialysis chambers. Next aliquots of 100 μL of the plasma spiked with test and reference compounds were applied to the donor side of the dialysis chambers. Blank buffer was added to the receiver first and the buffer and plasma chambers were clearly marked to avoid cross contamination.

Preparation of t=0 Min Plasma Samples for Initial Concentrations (Duplicate):

Aliquots of 25 μL of plasma spiked with test and reference compounds were placed in 96-well sample preparation plates as t=0 min plasma samples. The aliquots were mixed with the same volume of blank buffer (50:50, v/v). The samples were quenched with 200 μL of acetonitrile containing internal standard (IS). The dialysis block was covered with a plastic lid and the entire apparatus was placed on a shaker (60 rpm) for 5 hours at 37° C.

### Preparation of Dialyzed Samples After 5-Hour Incubation

25 μL aliquot from both the donor sides and receiver sides of the dialysis apparatus were placed into new sample preparation plates and mixed with the same valune of opposite matrixes (blank buffer to plasma and vice versa). The sample was quenched with 200 μL acetonitrile containing internal standard. All the samples from 0 hour to 5 hours were vortexed at 600 rpm for 10 minutes followed by centrifugation at 5594 g for 15 minutes (Thermo Multifuge×3R). 50 μL of the supernatant was transferred to a new 96-well plate and the sample was mized with 50 μL of Mili-Q water. The sample plate was covered and stored in a −20° C. freezer. The samples were subsequently analyzed by LC/MS/MS. The results are in Table 8.

### Solubility

10 μL test compound was added to 990 μL buffer. Sample tubes were shaken for 1 hour (1000 rpm) at room temperature. Samples were centrifuged for 10 minutes at 1200 rpm to precipitate un-dissolved particles. Supernatants were collected for LCMSMS or LC-UV analysis. The results are in Table 8.

### Thermodynamic Solubility

Assay buffer was added into compound powder to make 4 mg/ml solution. Sample tubes were shaken for 1 hour at 1000 rpm, then equilibrated over night at room temperature. Samples were centrifuged for 10 minutes at 12000 rpm to precipitate out un-dissolved particles. Supernatants were transferred to new tubes. Concentrations of the supernatants after centrifugation were determined by LCMSMS detection. The results are shown in Table 9.

### CYP Inhibition

0.1 M potassium phosphate buffer (K-buffer), pH 7.4 was preheated. K-buffer was prepared by mixing 9.5 mL Stock A into 40.5 mL Stock B. The total volume was brought to 500 mL with Milli-Q water. The buffer was titrated with KOH or H3PO4 to pH 7.4. Stock A (1 M monobasic potassium phosphate) was prepared by adding 136.5 g of monobasic potassium phosphate into 1 L of Milli-Q water. Stock B (1 M dibasic potassium phosphate) was prepared by adding 174.2 g of dibasic potassium phosphate in 1 L of Milli-Q water.

Serial dilution for test compound and reference inhibitors (400×) was prepared in a 96-well plate. 8 μL of 10 mM test compounds was transferred to 12 μL of ACN. Inhibitor spiking solution was prepared for CYP1A2, CYP2C9 and CYP2D6 in cocktail by adding 12 μL of 1 mM α-Naphthoflavon, 10 μL of 40 mM Sulfaphenazole, 10 μL of 10 mM Quinidine, and 8 μL of DMSO. Individual inhibitor spiking solution for CYP3A4 and CYP2C19 was prepared by adding 8 μL of DMSO stock to 12 μL of ACN. 1:3 serial dilutions were performed in DMSO:ACN mixture (v/v: 40:60).

NADPH cofactor was prepared by dissolving 66.7 mg NADPH in 10 mL 0.1 M K-buffer, pH7.4. 4×substrate (2 mL for each isoform) was prepared. HLM was added were required on ice. 0.2 mg/mL HLM solution was prepared using 10 μL of 20 mg/mL to 990 μL of 0.1 M K-buffer on ice. 400 μL of 0.2 mg/mL HLM were added to the assay wells and then 2 μL of 400×test compound set were added into the designated wells on ice. 200 μL of 0.2 mg/mL HLM was added to the assay wells and followed by 1 μL of serially diluted reference inhibtor solution into the designated wells on ice. The following solutions were added in duplicated in a 96-well plate on ice: 30 μL of 2×test compound and reference compound in 0.2 mg/mL HLM solution and 15 μL of 4×substrate solution. The 96-well assay plate and NADPH solution were pre-incubated at 37° C. for 5 minutes. 15 μL of pre-warmed 8 mM NADPH solution was added to into the assay plates to initiate the reaction. The assay plate was incubated at 37° C. 5 mins, 10 mins, and 45 mins. The reaction was stopped by adding 120 μL of ACN containing IS. After the reaction was quenched, the plates were shaken at the vibrator (IKA, MTS 2/4) for 10 min (600 rpm/min) and then centrifuge at 3220 g for 15 min. 50 μL of the supernatant was removed and transferred from each well into a 96-well sample plate containing 50 μL of ultra pure water (Millipore, ZMQS50F01) for LC/MS analysis. The results are shown in Table 9.

### Log D

10 μL of 10 mM stock solutions were placed into a 96-well plate. 300 μL of octanol into the plate was added to the plate. The plate was sealed and agitated on a plate shaker for 5 minutes. The plate was centrifuged at 2000 rpm for 5 minutes. The seal was removed and 600 μL of potassium phosphate buffer (pH 7.4) was added. The plate was sealed, and the two phases were mixed on the plate shaker vigorously for 1 hour at 25° C. The plate was centrifuged at 2000 rpm for 5 minutes after mixing and the seal was removed. 10 μL of water phase samples from the water phase plate was aspirated and mixed with 10 μL of water (1:2 dilution). 180 μL of 50% Ethanol was added into the water phase sample plate (1:10 dilution) to achieve 1:20 dilution. 50 μL of water phase sample was mixed with 100 μL of 50% Ethanol (containing IS) into the water phase sample plate to achieve 1:60 dilution. 10 μL from the octanol phase (the upper phase) was transfered into a new 96-well plate and 190 μL 50% Ethanol (1:20 dilution) was added. 10 μL of 1:20 diluted octanol samples were transferred into a new 96 deep-well plate and 390 μL 50% Ethanol (1:40 dilution) was added to achieve 1:800 dilution. 50 μL of 1:800 diluted octanol samples were transferred into a new 96-well plate again and 100 μL 50% ethanol with IS was added to achieve 1:2400 dilution. Samples were ready for LC/MS analysis. The results are shown in Table 9.

### Hepatocyte Stability

Vials with hepatocytes were removed from a liquid nitrogen storage unit and immediately placed in a 37±1° C. shaking water bath for 2 min±15 sec. The hepatocytes were poured into 50 mL of hepatocytes thawing medium, mixed gently, and centrifuged at 500 rpm for 3 minutes. After centrifugation, the supernatant was carefully aspirated without disturbing the pellet. 10× the volume of pre-warmed KHB buffer (Krebs-Henseleit buffer, Sigma Cat #K3753-10X1L) with 5.6 g/L HEPES was used to resuspend the cell pellet. The pellet mixture was centrifuged at 500 rpm for 3 minutes, aspirated, and the supernatant discarded without disturbing the cell pellet. The cell viability and yield were determined. The hepatocytes were counted, and the cell suspensions were diluted to the appropriate cell density. The viable cell density was 2×10⁶ cells/mL. The hepatocyte solution was placed on ice until it was used.

Two dosing solutions of prewarmed KHB (1% DMSO) were prepared. 200 μM spiking solution was prepared by adding 20 μL of substrates stock solution (10 mM) into 980 μL of DMSO. Two dosing solutions were prepared by adding 10 μL of 200 μM spiking solution into 990 μL of KHB (2 μM after dilution). The dosing solution was centrifuged at 5594 g for 15 min (Thermo Multifuge×3R). 50 μL of pre-warmed 2×dosing solution was added to the wells designated for the different time points. 50 μL of pre-warmed hepatocytes solution (2×10⁶ cells/mL) was added to the wells designateed for 15 min, 30 min, 60 min, and 120 min. After the addition of the hepatocytes, the timer was started. The assay was placed in an incubator at 37° C. 100 μL of ACN containing IS was added to the wells designed for 0 min, the wells were mixed gently, then 50 μL of pre-warmed hepatocytes solution (2×10⁶ cells/mL) was added. The wells were sealed. At 15 min, 30 min, 60 min, and 120 min, 100 μL of ACN containing IS was added to the wells, respectively. Next, the wells were sealed. After quenching, the plates were shaken on a vibrator (IKA, MTS 2/4) for 10 min at 600 rpm. The plate was sonicated for 2 min and centrifuged at 5594 g for 15 min (Thermo Multifuge×3R). 50 μL of the supernatant was transferred from each well into a 96-well sample plate containing 50 μL of ultra pure water (Millipore, ZMQS50F01) for LC/MS analysis. The results are shown in Table 9.

### RBC Partitioning Study

0.05 M sodium phosphate buffer, pH 7.4 was prepared using the following steps. Stock A, stock B, and stock C solutions were prepared by adding 156 g of monobasic sodium phosphate in 1 L of Milli-Q water (1.0 M), 142 g of dibasic sodium phosphate in 1 L of Milli-Q water (1.0 M), and 58.5 g of sodium chloride in 1 L of Milli-Q water (1.0 M) respectively. 20.25 mL of Stock B and 4.75 mL of Stock A were added to 35 mL of Stock C and the total volume was brought to near 500 mL with Milli-Q water. The resulting buffer solution was titrated with NaOH or H₃PO₄ to pH 7.4 and the total volume was brought to 500 mL. The 0.05 M sodium phosphate buffer was preheated at 37° C.

Fresh blank plasma preparation and hematocrit determination. A tube containing whole blood was centrifuged at 4000 rpm for 10 minuts. The plasma was harvested was stored. Hematocrit (the volume of red blood cells to the total volume of whole blood) was determined using the formula:

Hematocrit=Vred cells/Vblood=(Vblood−Vplasma)/Vblood

Vblood: the volume of whole blood applied

Vplasma: the volume of plasma measured after centrifugation

Preparation of reference and test compound spiking solutions in plasma. 1.25 mM spiking solution A was prepared by adding add 10 μL of 10 mM reference into 70 μL of plasma (8-fold dilution). 125 μM spiking solution B was prepared by adding 10 μL of 1.25 mM spiking solution into 90 μL of plasma (10-fold dilution).

Preparation of reference and test compound in whole blood and plasma. For whole blood samples, 12 μL of spiking solution B (prepared above) was added to 288 μL of whole blood (25-fold dilution). For plasma samples, 6 μL of spiking solution B was added to 144 μL of plasma (25-fold dilution). The assay procedure used is as follows. The assay plate was placed in an incubator at 37° C. for 60 minutess. 300 μL of compound spiking solution in whole blood from above was removed and centrifuged at 4000 rpm for 10 minutes. Subsequently plasma was harvested. 50 μL of plasma sample was removed and 150 μL of acetonitrile (containing IS) was added and vortexed for 5 min. The sample was centrifuged at 6000 rpm for 15 minutes. The supernatant was removed and analyzed by LC/MS.

TABLE 8
Chemical stability, metabolic stability, protein binding,
and solubility data from in vitro ADME study results

| | | | | | | | Protein Binding | |
|---|---|---|---|---|---|---|---|---|
| Chemical Stability | | | | Metabolic stability | | | | Fraction |
| Test | Test | | Sample | | | Clint | | of |
| Article | System | T½(min) | Size | Species | T½(min) | (mL/min/kg) | Species | bound(%) |
| 7 | PBS(Ph 7.4) | ∞ | 2 | rat | 134.73 | 18.44 | rat | 62.2 |
| | 10 um | | | | | | | |
| 9 | PBS(Ph 7.4) | ∞ | 2 | rat | 1432.2 | 1.73 | rat | 59.1 |
| | 10 um | | | | | | | |
| 8 | PBS(Ph 7.4) | 4875.07 | 2 | rat | 582.88 | 2.98 | rat | 71.835804 |
| | 10 um | | | human | 84.14 | 29.52 | | |

| Chemical Stability | Protein Binding | | | | Solubility | |
|---|---|---|---|---|---|---|
| Test | | | Recovery | | Test | SolubilityU |
| Article | Fu(%) | Category | (%) | Comment | System | (um) |
| 7 | 37.8 | Moderate | 96.6 | NA | PBS(Ph 7.4) | 101.75 ± 0.03 |
| 9 | 40.9 | Moderate | 68.2 | Could be | PBS(Ph 7.4) | 130.50 ± 0.02 |
| | | | | poor | | |
| | | | | stability | | |
| | | | | in plasma | | |
| 8 | 28.164196 | Moderate | 102.74223 | NA | PBS(Ph 7.4) | 91.6 |

TABLE 9
Thermodynamic stability, CYP inhibition, LogD, hepatocyte stability and RBC partitioning data in vitro ADME study results

| | | | | | | | | | | Hepatocyte | | | | | |
|---|---|---|---|---|---|---|---|---|---|---|---|---|---|---|---|
| Thermodynamic | | | | | | | | | | Stability | | | RBC Partitioning | | |
| Solubility | | | CYP inhibition | | | | | | | | | Clint | Study | | |
| Test | Solubility | Solubility | (IC50 um) | | | | | LogD | | | T½ | (mL/kg/ | | KRBC/ | KBlood/ |
| System | (um) | (ug/mL) | 1A2 | 2C9 | 2C19 | 2D6 | 3A | Mean | RSD | Spcies | (min) | min) | Spcies | PL | PL |
| PBS(Ph 7.4) | 4435 | 1680.2 | >10 | >10 | >10 | >10 | >10 | −0.07 | −3.01 | rat | 600.12 | 5.4 | rat | 1.15 | 1.09 |
| PBS(Ph 7.4) | 5995 | 2247.6 | >10 | >10 | >10 | >10 | >10 | −0.11 | −1.15 | rat | 1023.63 | 3.17 | rat | 1.09 | 1.05 |
| PBS(Ph 7.4) | 4920 | 2082.6852 | >10 | >10 | >10 | >10 | >10 | −0.15 | −0.11 | rat | 539.74 | 6.01 | rat | 0.94 | 0.96 |

### Example 29: hERG Study Results

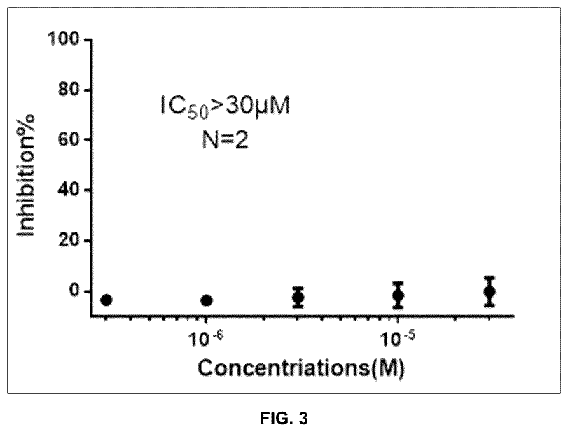

Compound 8 was dissolved in DMSO, with a final concentration of DMSO in the extracellular fluid not exceeding 0.3%. The final concentrations of Compound 8 were 0.3, 1, 3, 10, and 30 μM. The inhibitory effect of Compound 8 on hERG current is presented in Table 10, and the concentration-response curve is depicted in FIG. 3. Under the conditions of this experiment, compound 8 showed no significant inhibitory effect on the hERG potassium channel current. At 30 μM, the average inhibition rate on the hERG current was −0.15% (N=2).

TABLE 10
Inhibitory Effects of Compound 8 on hERG Current

| | Inhibition Rate (%) | | | | |
|---|---|---|---|---|---|
| Cell No. | 0.3 μM | 1 μM | 3 μM | 10 μM | 30 μM |
| Cell No. 1 | −3.35 | −5.07 | −4.96 | −5.05 | −4.07 |
| Cell No. 2 | −3.63 | −2.28 | 0.08 | 1.72 | 3.77 |
| Mean | −3.49 | −3.67 | −2.44 | −1.66 | −0.15 |
| SD | 0.20 | 1.97 | 3.57 | 4.79 | 5.54 |

What is claimed is:

1. A compound of Formula (I), or stereoisomer, pharmaceutically acceptable salt, solvate, deuterate, metabolite, or prodrug thereof; 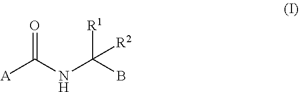
wherein
R¹ and R² are independently selected from H, deuterium, C1-C6 alkyl, substituted C1-C6 alkyl, C3-C6 cycloalkyl, C4-C9 alkylcycloalkyl, C2-C6 alkenyl, substituted C2-C6 alkenyl, aryl, substituted aryl, alkylaryl, or substituted alkylaryl;
wherein the substituted C1-C6 alkyl, substituted C3-C6 cycloalkyl, and substituted aryl are substituted with 1-3 substituents independently selected from the following groups: C1-C3 alkyl, C1-C3 alkoxy, halogen, or halogenated C1-C3 alkyl; or
R¹ and R² are connected to form three-member, four-member, five-member and six-member rings;
A is selected from the following 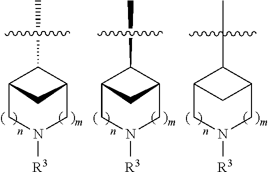
wherein
n=1, 2, 3, 4;
m=1, 2, 3, 4;
R³ is selected from the group consisting of H, deuterium, C1-C6 alkyl, oxygen-containing alkyl, and nitrogen-containing alkyl;
B is selected from the group consisting of aromatic ring, substituted aromatic ring, heterocycle, substituted heterocycle, alkyl heterocycle, and substituted alkyl heterocycle;
wherein the substituted heterocycle or alkyl heterocycle is substituted by 1-3 independently selected from the group consisting of deuterium, halogen, cyano, C1-C6 alkyl, halogenated C1-C6 alkyl, C3-C6 cycloalkyl, C1-C3 alkoxy, alkylsilyl, aryl, substituted aryl, or oxygen, sulfur, selenium-containing alkyl.

2. The compound of claim 1, wherein R¹ and R² are independently selected from H, deuterium, methyl, ethyl, propyl, and butyl.

3. The compound of claim 1, wherein R¹ and R² are independently selected from cyclopropyl, cyclobutyl base, cyclopentyl, cyclopropylmethyl, cyclopropylethyl; R¹ and R² can also be connected to form three-member, four-member, five-member and six-member rings.

4. The compound of claim 1, wherein R¹ and R² are independently selected from vinyl, propenyl, allyl, phenyl, naphthyl, phenylmethyl, phenylethyl; R¹ and R² can also be connected to form three-member, four-member, five-member, and six-member rings.

5. The compound of claim 1, wherein R¹ and R² are independently selected from substituted C1-C6 alkyl, substituted C3-C6 cycloalkyl, substituted C2-C6 alkenyl, substituted aryl, substituted alkylaryl;
wherein the substituent is selected from the following groups methyl, ethyl, propyl, methoxy, ethoxy, fluorine, chlorine, trifluoromethyl; or
R¹ and R² are connected to form three-member, four-member, five-member and six-member rings.

6. The compound of claim 1, wherein A is selected from the following: 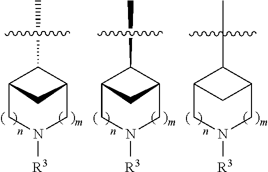
wherein
n=1, m=1;
R³ is selected from H, deuterium, C1-C6 alkyl, oxygen-containing alkyl, and nitrogen-containing alkyl;
wherein the C1-C6 alkyl is independently selected from methyl, ethyl, propyl, and butyl;
wherein the oxygen-containing alkyl is independently selected from aldehyde group, ester group, and carboxyl group; and
wherein the nitrogen-containing alkyl is independently selected from alkylamino group, amide group, and amidine group.

7. The compound of claim 1, wherein B is selected from an aromatic ring, substituted aromatic ring, heterocycle, substituted heterocycle, alkyl heterocycle, substituted alkyl heterocycle; wherein the aromatic ring is independently selected from phenyl and naphthyl; and the heterocycle, alkyl heterocycle is independently selected from furyl, benzofuryl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidinyl, pyrazinyl, pyridazinyl, indolyl, isoindolyl, indolizinyl, benzimidazolyl, indazolyl, benzotriazolyl, tetrazopyridazinyl, carbazolyl, purinyl, quinolyl, isoquinolyl, imidazopyridyl, or other fused heterocycles.

8. The compound of claim 1, wherein B is selected from a substituted aromatic ring, substituted heterocyclic ring, substituted alkyl heterocycle; wherein the substituent is independently selected from fluorine, chlorine, bromine, cyano, methyl, ethyl, propyl, butyl, trifluoromethyl, cyclopropyl, cyclobutyl, cyclopentyl, C1-C9 alkoxy group, alkylsilyl group, phenyl group, naphthyl group, substituted aryl group, or thionyl group.

9. The compound of claim 1, wherein the compound comprises the Formula (Ia) 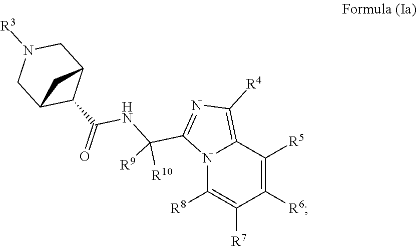
wherein
R³ is hydrogen or amidine;
R⁴-R⁸ are independently hydrogen, halogen, C1-C6 alkyl, substituted C1-C6 alkyl, C1-C3 alkoxy, C3-C6 cycloalkyl, aryl, substituted aryl, sulfinyl, substituted sulfinyl, or C1-C3 alkyl silyl group;
wherein the substituted alkyl is substituted by 1-3 independently selected from the group consisting of deuterium, halogen, cyano, C1-C6 alkyl, halogenated C1-C6 alkyl, C3-C6 cycloalkyl, C1-C3 alkoxy, alkylsilyl, aryl, substituted aryl, or oxygen, sulfur, selenium-containing alkyl; and
R³-R⁸ are independently hydrogen or C1-C6 alkyl.

10. The compound of claim 1, wherein the compound comprises the Formula (Ib) 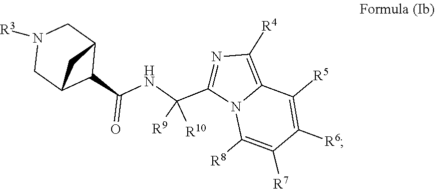
wherein
R³ is hydrogen or amidine;
R⁴-R⁸ are independently hydrogen, halogen, C1-C6 alkyl, substituted C1-C6 alkyl, C1-C3 alkoxy, C3-C6 cycloalkyl, aryl, substituted aryl, sulfinyl, substituted sulfinyl, or C1-C3 alkyl silyl group;
wherein the substituted alkyl is substituted by 1-3 independently selected from the group consisting of deuterium, halogen, cyano, C1-C6 alkyl, halogenated C1-C6 alkyl, C3-C6 cycloalkyl, C1-C3 alkoxy, alkylsilyl, aryl, substituted aryl, or oxygen, sulfur, selenium-containing alkyl; and
R³-R⁸ are independently hydrogen or C1-C6 alkyl.

11. The compound of formula (I) as claimed in claim 1, wherein the compound of formula (I) is selected from any of the following compounds or pharmaceutically acceptable salts thereof:

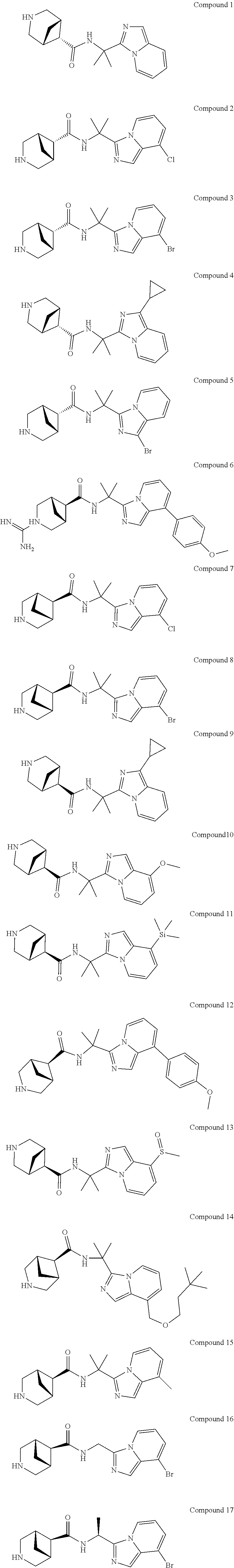

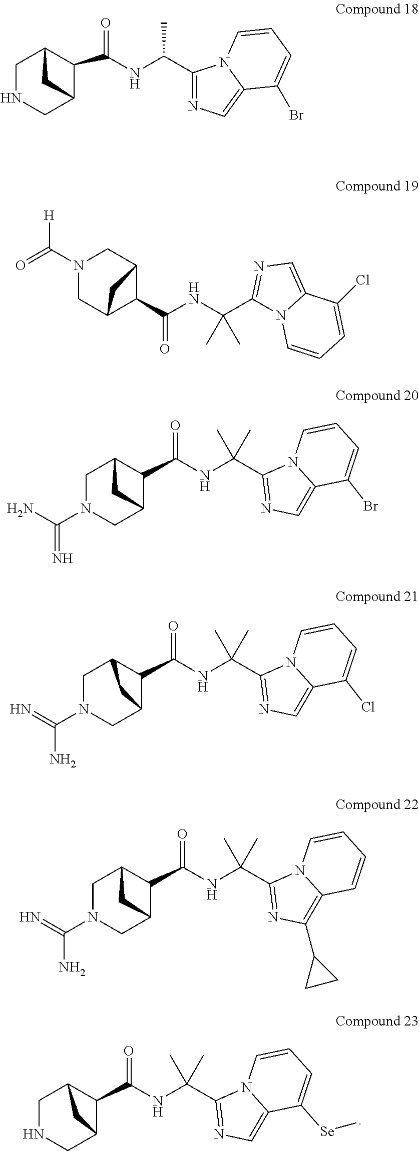

12. A pharmaceutical composition comprising the compound of claim 1 or its stereoisomer, pharmaceutically acceptable salt, solvate, deuterated compound, metabolite, or prodrug, and a pharmaceutically acceptable carrier.

13. A method of treating and/or preventing a SSTR4 receptor-related disease or disorder, the method comprising administering a pharmaceutically effective amount of the pharmaceutical composition of claim 12.

14. The method of claim 13, wherein the SSTR4 receptor-related disease or disorder is pain and/or prevention of pain.

15. The method of claim 14, wherein the pain and/or prevention of pain is related to SSTR4 receptor.

16. The method of claim 14, wherein the SSTR4 receptor-related disease or disorder is neuropathid pain, visceral pain, or combinations thereof.

17. A method of making a compound of Formula (I), the method comprising:
reacting a first compound with a protecting group and carboxylic acid group with a second compound with an amine group to form a first intermediate compound;
deprotecting the protecting group in the first intermediate compound under acidic conditions to form a second intermediate; and
reacting the second intermediate with an amine compound forming the compound of Formula (I).
